(19) United States
(12) Patent Application Publication
(10) Pub. No.: US 2010/0216791 A1
(43) Pub. Date: Aug. 26, 2010

(54) PYRIDINYLQUINAZOLINAMINE DERIVATIVES AND THEIR USE AS B-RAF INHIBITORS

(75) Inventors: Brian Aquila, Waltham, MA (US); Donald J. Cook, Waltham, MA (US); Craig Johnstone, Macclesfield (GB); Stephen Lee, Waltham, MA (US); Paul Lyne, Waltham, MA (US); David Alan Rudge, Macclesfield (GB); Melissa Vasbinder, Waltham, MA (US); Haixia Wang, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA, Södertälje (SE)

(21) Appl. No.: 12/377,285

(22) PCT Filed: Aug. 15, 2007
(86) PCT No.: PCT/GB2007/003111

§ 371 Date: Feb. 12, 2009

Related U.S. Application Data
(60) Provisional application No. 60/822,749, filed on Aug. 17, 2006.
(60) Provisional application No. 60/887,062, filed on Jan. 29, 2007.

(51) Int. Cl. A61K 31/5377; C07D 401/04; A61K 31/517; A61P 35/00; A61P 35/02; C07D 413/14

(52) U.S. Cl. 514/234.5; 544/284; 514/266.21; 544/119

(57) ABSTRACT

The invention relates to chemical compounds of the formula (I) or pharmaceutically acceptable salts thereof, which possess B-Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

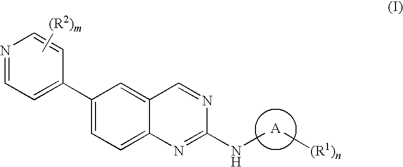

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal—regulated kinase kinase (MEK), extracellular signal—regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 Apr., http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemann and Rapp, Expt. Cell Res. 1999, 253, 34-46). Expression of all three Raf genes is required for normal murine development however both c-Raf and B-Raf are required to complete gestation. B-Raf−/− mice die at E12.5 due to vascular haemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca has filed certain international applications directed towards B-Raf inhibitors: PCT publication Nos. WO 2005/123696, WO 2006/003378, WO 2006/024834, WO 2006/024836, WO 2006/040568, WO 2006/067446 and WO 2006/079791.

Amgen's PCT publication WO 2006/039718, published 13 Apr. 2006, describes aryl nitrogen-containing bicyclic compounds for use in treating protein kinase mediated disease states, including inflammation, cancer and related conditions.

The present application is based on a class of compound which are novel B-Raf inhibitors and it is expected that these compound could possess beneficial efficacious, metabolic and/or toxicological profiles that make them particularly suitable for in vivo administration to a warm blooded animal, such as man.

Accordingly, the present invention provides a compound of formula (I):

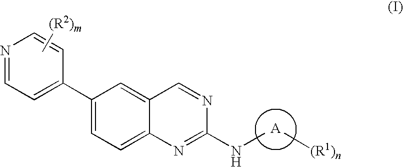

wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N′—(C₁₋₆alkyl)ureido, N′,N′—(C₁₋₆alkyl)₂ureido, N′—(C₁₋₆alkyl)-N—(C₁₋₆alkyl)ureido, N′,N′—(C₁₋₆alkyl)₂-N—(C₁₋₆alkyl)ureido, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, (R²¹)(R²²)P(O)—, (R²⁹)(R³⁰)P(O)NH—, (R³¹)(R³²)P(O)N(C₁₋₆alkyl)-, (R²⁵)(R²⁶)(R²⁷)Si—, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

n is selected from 0-4; wherein the values of R¹ may be the same or different;

R² is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;

m is selected from 0-4; wherein the values of R² may be the same or different;

R⁶ and R¹⁰ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)-N—(C₁₋₆alkoxycarbonyl)amino, N—(C₁₋₆alkyl)sulphamoyl, N,N—(C₁₋₆alkyl)₂sulphamoyl, C₁₋₆alkylsulphonylamino, (R²³)(R²⁴)P(O)—, (R³³)(R³⁴)P(O)NH—, (R³⁵)(R³⁶)P(O)N(C₁₋₆ alkyl)-, carbocyclyl-R¹²— or heterocyclyl-R¹³—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁴;

R²¹, R²², R²³, R²⁴, R²⁹, R³⁰, R³¹, R³², R³³, R³⁴, R³⁵ and R³⁶ are independently selected from amino, C₁₋₆alkyl, C₁₋₆alkoxy and carbocyclyl;

R²⁵, R²⁶ and R²⁷ are independently selected from hydroxy, C₁₋₆alkyl, C₁₋₆alkoxy and carbocyclyl; or R²⁵ and R²⁶ together with the silicon to which they are attached form a ring; wherein R²⁵, R²⁶ and R²⁷ may be independently optionally substituted on carbon by one or more R²⁸;

R⁴, R⁵, R⁸, R⁹, R¹² and R¹³ are independently selected from a direct bond, —O—, —N(R¹⁶)—, —N(R¹⁷)C(O)—, —C(O)N(R¹⁸)—, —S(O)ₛ—, —SO₂N(R¹⁹)— or —N(R²⁰)SO₂—; wherein R¹⁶, R¹⁷, R¹⁸, R¹⁹ and R²⁰ are independently selected from hydrogen, C₁₋₆alkoxycarbonyl or C₁₋₆alkyl and s is 0-2;

R³, R⁷, R¹¹ and R¹⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁵ and R²⁸ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, carbocyclyl or heterocyclyl; wherein if said heterocyclyl contains an —NH— moiety that nitrogen may be optionally substituted by methyl;

or a pharmaceutically acceptable salt thereof.

According to a further feature of the present invention there is provided a compound of formula (I) wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆ alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

n is selected from 0-4; wherein the values of R¹ may be the same or different;

R² is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁶; and wherein if said heterocyclyl contains an —NH— moiety that nitrogen may be optionally substituted by a group selected from R¹¹;

m is selected from 0-4; wherein the values of R² may be the same or different;

R⁶ and R¹⁰ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹²— or heterocyclyl-R¹³—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁴;

R⁴, R⁵, R⁸, R⁹, R¹² and R¹³ are independently selected from a direct bond, —O—, —N(R¹⁶)—, —N(R¹⁷)C(O)—, —C(O)N(R¹⁸)—, —SO₂N(R¹⁹)— or —N(R²⁰)SO₂—; wherein R¹⁶, R¹⁷, R¹⁸, R¹⁹ and R²⁰ is hydrogen, C₁₋₆alkoxycarbonyl or C₁₋₆alkyl and s is 0-2;

R³, R⁷, R¹¹ and R¹⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁵ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

According to a further feature of the present invention there is provided a compound of formula (I) wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N′—(C₁₋₆alkyl)ureido, N′,N′—(C₁₋₆alkyl)₂ureido, N′—(C₁₋₆alkyl)-N—(C₁₋₆alkyl)ureido, N′,N′—(C₁₋₆alkyl)₂-N—(C₁₋₆alkyl)ureido, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

n is selected from 0-4; wherein the values of R¹ may be the same or different;

R² is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;

m is selected from 0-4; wherein the values of R² may be the same or different;

R⁶ and R¹⁰ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)-N—(C₁₋₆alkoxycarbonyl)amino, N—(C₁₋₆alkyl)sulphamoyl, N,N—(C₁₋₆alkyl)₂sulphamoyl, C₁₋₆alkylsulphonylamino, carbocyclyl-R¹²— or heterocyclyl-R¹³—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁴;

R⁴, R⁵, R⁸, R⁹, R¹² and R¹³ are independently selected from a direct bond, —O—, —N(R¹⁶)—, —C(O)—, —N(R¹⁷)C(O)—, —C(O)N(R¹⁸)—, —S(O)ₛ—, —SO₂N(R¹⁹)— or —N(R²⁰)SO₂—; wherein R¹⁶, R¹⁷, R¹⁸, R¹⁹ and R²⁰ is hydrogen, C₁₋₆alkoxycarbonyl or C₁₋₆alkyl and s is 0-2;

R³, R⁷, R¹¹ and R¹⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁵ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N-diethylsulphamoyl, N-methyl-N-ethylsulphamoyl, carbocyclyl or heterocyclyl;

or a pharmaceutically acceptable salt thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. Additional examples and suitable values of the term “heterocyclyl” are pyrrolidinyl, 1,3-benzodioxolyl, 2,3-dihydro-1,4-benzodioxinyl, benzooxazolyl, pyrazolyl, 2-oxopyrrolidinyl, piperazinyl, morpholino, piperidinyl, piperidinyl imidazolyl, pyridyl, furanyl, 1,3-dioxolanyl or 1,4-diazepanyl.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl. A further example of “carbocyclyl” is cyclopropyl.

In one aspect of the invention “R²⁵ and R²⁶ together with the silicon to which they are attached form a ring”. In such an aspect, R²⁵ and R²⁶ together may form, for example, a C₂₋₅alkylene group or a —OC₂₋₅alkyleneO— (for example —O(CH₂)₂O—) group. A suitable example is 1,3,2-dioxasilolan-2-yl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include formyl, propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₆alkyl)carbamoyl, methylaminocarbonyl and ethylaminocarbonyl. Examples of “N,N—(C₁₋₆alkyl)₂carbamoyl” are N,N—(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino. Examples of “N—(C₁₋₆alkoxy)sulphamoyl” include N-(methoxy)sulphamoyl and N-(ethoxy)sulphamoyl. Examples of “N—(C₁₋₆alkyl)-N—(C₁₋₆alkoxy)sulphamoyl” include N-(methyl)-N-(methoxy)sulphamoyl and N-(propyl)-N-(ethoxy)sulphamoyl. Examples of “N—(C₁₋₆alkyl)-N—(C₁₋₆alkanoyl)amino” include N-(methyl)-N-(acetyl)amino and N-(propyl)-N-(propionyl)amino. Examples of “N—(C₁₋₆alkyl)-N—(C₁₋₆alkoxycarbonyl)amino” include N-(methyl)-N-(methoxycarbonyl)amino and N-(propyl)-N-(t-butoxycarbonyl)amino.

“(R²¹)(R²²)P(O)—” represents a compound of the following structure:

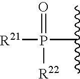

similar groups to the above wherein the R group is different represent the corresponding structure.

“(R²⁵)(R²⁶)(R²⁷)Si—” represents a compound of the following structure:

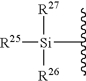

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is carbocyclyl or heterocyclyl; wherein if said heterocyclyl contains an —NH— moiety that nitrogen may be optionally substituted by a group selected from R³; wherein R³ is selected from C₁₋₆alkyl.

Ring A is phenyl, pyridyl, 1,3-benzodioxolyl, 2,3-dihydro-1,4-benzodioxinyl or pyrazolyl; wherein said pyrazolyl may be optionally substituted on nitrogen by a group selected from R³; wherein R³ is selected from C₁₋₆alkyl.

Ring A is phenyl, pyridyl, 1,3-benzodioxolyl, 2,3-dihydro-1,4-benzodioxinyl, benzooxazolyl or pyrazolyl; wherein said pyrazolyl may be optionally substituted on nitrogen by a group selected from R³; wherein R³ is selected from methyl.

Ring A is phenyl, pyridyl, 1,3-benzodioxolyl, 2,3-dihydro-1,4-benzodioxinyl or pyrazolyl; wherein said pyrazolyl may be optionally substituted on nitrogen by a group selected from R³; wherein R³ is selected from methyl.

Ring A is phenyl, pyrid-2-yl, pyrid-3-yl, 1,3-benzodioxol-5-yl, 2,3-dihydro-1,4-benzodioxin-6-yl, benzooxazol-5-yl or 1-methylpyrazol-3-yl.

Ring A is phenyl, pyrid-2-yl, pyrid-3-yl, 1,3-benzodioxol-5-yl, 2,3-dihydro-1,4-benzodioxin-6-yl or 1-methylpyrazol-3-yl.

Ring A is heterocyclyl; wherein if said heterocyclyl contains an —NH— moiety that nitrogen may be optionally substituted by a group selected from R³.

Ring A is heterocyclyl.

Ring A is carbocyclyl.

Ring A is phenyl.

R¹ is a substituent on carbon and is selected from halo, hydroxy, amino, carboxy,

C₁₋₆alkyl, C₁₋₆alkoxy, C₁₋₆alkanoyl, N,N—(C₁₋₆alkyl)₂amino, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 2, C₁₋₆alkoxycarbonyl, N—(C₁₋₆alkyl)sulphamoyl or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷; wherein

R⁶ is selected from halo, cyano, hydroxy, amino, C₁₋₆alkoxy, N—(C₁₋₆alkyl)amino, N,N—(C₁₋₆alkyl)₂amino, C₁₋₆alkanoylamino, C₁₋₆alkoxycarbonylamino, N—(C₁₋₆alkyl)-N—(C₁₋₆alkoxycarbonyl)amino, (R³⁵)(R³⁶)P(O)N(C₁₋₆alkyl)- or heterocyclyl-R¹³—; wherein R⁶ may be optionally substituted on carbon by one or more R¹⁵; and wherein if said heterocyclyl contains an —NH— moiety that nitrogen may be optionally substituted by a group selected from R¹⁴;

R³⁵ and R³⁶ are independently selected from C₁₋₆alkyl;

R⁵ and R¹³ are independently selected from a direct bond, —C(O)—, —C(O)N(R¹⁸)— or —S(O)ₛ—; wherein R¹⁸ is hydrogen and s is 0-2;

R⁷ and R¹⁴ are independently selected from C₁₋₆alkyl, C₁₋₆alkanoyl and C₁₋₆alkoxycarbonyl; and

R¹⁵ is selected from hydroxy, methyl, methoxy, dimethylamino, carbocyclyl or heterocyclyl; wherein if said heterocyclyl contains an —NH— moiety that nitrogen may be optionally substituted by methyl.

R¹ is a substituent on carbon and is selected from halo, hydroxy, C₁₋₆alkyl, C₁₋₆alkoxy, N,N—(C₁₋₆alkyl)₂amino, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 2, C₁₋₆alkoxycarbonyl, N—(C₁₋₆alkyl)sulphamoyl or heterocyclyl-R⁵—; wherein R¹ may be optionally substituted on carbon by one or more R⁶; wherein

R⁶ is selected from halo, cyano, hydroxy, amino, C₁₋₆alkoxy, N—(C₁₋₆alkyl)amino, N,N—(C₁₋₆alkyl)₂amino, C₁₋₆alkoxycarbonylamino, N—(C₁₋₆alkyl)-N—(C₁₋₆alkoxycarbonyl)amino or heterocyclyl-R¹³—; wherein R⁶ may be optionally substituted on carbon by one or more R¹⁵; and wherein if said heterocyclyl contains an —NH— moiety that nitrogen may be optionally substituted by a group selected from R¹⁴;

R⁵ and R¹³ are independently selected from a direct bond, —N(R¹⁷)C(O)— or —S(O)ₛ—; wherein R¹⁷ is hydrogen and s is 2;

R¹⁴ is selected from C₁₋₆alkyl or C₁₋₆alkoxycarbonyl; and

R¹⁵ is selected from hydroxy, methyl, methoxy or heterocyclyl.

R¹ is a substituent on carbon and is selected from C₁₋₆alkyl or N—(C₁₋₆alkyl)carbamoyl; wherein R¹ may be optionally substituted on carbon by one or more R⁶; wherein

R⁶ is selected from cyano, C₁₋₆alkoxy or heterocyclyl-R¹³—; and

R¹³ is selected from a direct bond.

R¹ is a substituent on carbon and is selected from fluoro, chloro, hydroxy, amino, carboxy, methyl, ethyl, isopropyl, methoxy, ethoxy, isopropoxy, formyl, acetyl, dimethylamino, acetylamino, propanoylamino, N-methyl-N-acetylamino, N-methyl-N-propanoylamino, N-methylcarbamoyl, N-ethylcarbamoyl, N-propylcarbamoyl, N-isopropylcarbamoyl, N-butylcarbamoyl, dimethylcarbamoyl, diethylcarbamoyl, N-methyl-N-ethylcarbamoyl, mesyl, methoxycarbonyl, N-ethylsulphamoyl, N-propylsulphamoyl, pyrrolidin-1-yl-R⁵—, pyrazol-1-yl-R⁵—, 2-oxopyrrolidin-1-yl-R⁵—, piperazin-1-yl-R⁵—, morpholino-R⁵—, piperidin-4-yl-R⁵— or piperidin-1-yl-R⁵—; wherein R¹ may be optionally substituted on carbon by one or more R⁶; and wherein if said heterocyclyl contains an —NH— moiety that nitrogen may be optionally substituted by a group selected from R⁷; wherein

R⁶ is selected from fluoro, cyano, hydroxy, amino, methoxy, ethoxy, methylamino, ethylamino, N-propylamino, N-isopropylamino, N-butylamino, dimethylamino, diethylamino, N-methyl-N-ethylamino, N-methyl-N-propylamino, acetylamino, propanoylamino, t-butoxycarbonylamino, N-methyl-N-t-butoxycarbonylamino, (R³⁵)(R³⁶)P(O)N(methyl)-, imidazol-1-yl-R¹³—, pyrid-2-yl-yl-R¹³—, pyrrolidin-1-yl-R¹³—, pyrrolidin-2-yl-R¹³—, 2-oxopyrrolidin-1-yl-R¹³—, furan-2-yl-R¹³—, 1,3-dioxolan-4-yl-R¹³—, piperidin-1-yl-R¹³—, piperidin-4-yl-R¹³—, piperidin-2-yl-R¹³—, piperazin-2-yl-R¹³—, 1,4-diazepan-1-yl-R¹³— or morpholino-R¹³—; wherein R⁶ may be optionally substituted on carbon by one or more R¹⁵; and wherein if said heterocyclyl contains an —NH— moiety that nitrogen may be optionally substituted by a group selected from R¹⁴;

R³⁵ and R³⁶ are methyl;

R⁵ and R¹³ are independently selected from a direct bond, —C(O)—, —C(O)N(R¹⁸)— or —S(O)ₛ—; wherein R¹⁸ is hydrogen and s is 0-2;

R⁷ and R¹⁴ are independently selected from methyl, acetyl and t-butoxycarbonyl; and

R¹⁵ is selected from hydroxy, methyl, methoxy, dimethylamino, cyclopropyl, pyrrolidin-2-yl or morpholino; wherein said pyrrolidinyl may be optionally substituted on nitrogen by methyl.

R¹ is a substituent on carbon and is selected from fluoro, chloro, hydroxy, methyl, isopropyl, methoxy, ethoxy, isopropoxy, diethylamino, acetylamino, N-methyl-N-acetylamino, N-methylcarbamoyl, N-ethylcarbamoyl, N-propylcarbamoyl, N-isopropylcarbamoyl, N-butylcarbamoyl, N,N-dimethylcarbamoyl, mesyl, methoxycarbonyl, N-butylsulphamoyl, piperidinyl-R⁵—, pyrrolidinyl-R⁵— or morpholino-R⁵—; wherein R¹ may be optionally substituted on carbon by one or more R⁶; wherein

R⁶ is selected from fluoro, cyano, hydroxy, amino, methoxy, ethoxy, methylamino, ethylamino, isopropylamino, dimethylamino, t-butoxycarbonylamino, N-methyl-N-(t-butoxycarbonyl)amino, pyrrolidinyl-R¹³—, piperidinyl-R¹³—, imidazolyl-R¹³—, 2-oxopyrrolidinyl-R¹³—, 1,3-dioxolanyl-R¹³—, pyridyl-R¹³—, tetrahydrofuranyl-R¹³— or morpholino-R¹³—; wherein R⁶ may be optionally substituted on carbon by one or more R¹⁵; and wherein said pyrrolidinyl or piperidinyl may be optionally substituted on nitrogen by a group selected from R¹⁴;

R⁵ and R¹³ are independently selected from a direct bond, —N(R¹⁷)C(O)— or —S(O)ₛ—; wherein R¹⁷ is hydrogen and s is 2;

R¹⁴ is selected from methyl or t-butoxycarbonyl; and

R¹⁵ is selected from hydroxy, methyl, methoxy or morpholino.

R¹ is a substituent on carbon and is selected from isopropyl or N-(ethyl)carbamoyl; wherein R¹ may be optionally substituted on carbon by one or more R⁶; wherein

R⁶ is selected from cyano, methoxy or morpholino-R¹³—; and

R¹³ is selected from a direct bond.

R¹ is a substituent on carbon and is selected from
(1R)-1-(3-methoxypropanoylamino)ethyl, (1R)-1-acetamidoethyl,
(1R)-1-dimethylaminoethyl, (1S)-1-(3-methoxypropanoylamino)ethyl, (1S)-1-acetamidoethyl,
(1S)-1-dimethylaminoethyl, (1-tert-butoxycarbonyl-4-piperidyl)methylcarbamoyl,
(1-tert-butoxycarbonylpyrrolidin-2-yl)methylcarbamoyl,
(2,2-dimethyl-1,3-dioxolan-4-yl)methylcarbamoyl, (2-dimethylaminoethylamino)methyl,
(2-hydroxyethyl-methyl-amino)methyl, (2-methoxy-1-methyl-ethyl)carbamoyl,
(2-methoxyethylamino)methyl, (2-methoxyethyl-methyl-amino)methyl,
(2-morpholinoethylamino)methyl, (3S)-3-dimethylaminopyrrolidine-1-carbonyl,
(4-methylpiperazin-1-yl)methyl, (ethyl-(2-hydroxyethyl)amino)methyl,
[(2-dimethylamino-1-methyl-ethyl)amino]methyl,
[2-(1-methylpyrrolidin-2-yl)ethylamino]methyl, 1-(2-hydroxyethylamino)ethyl,
1-(2-hydroxyethyl-methyl-amino)ethyl, 1-(2-methoxyethylamino)ethyl,
1-(3-hydroxybutylamino)ethyl, 1-(3-hydroxypropylamino)ethyl,
1-(3-hydroxypropyl-methyl-amino)ethyl, 1-(3-methoxypropanoylamino)ethyl,
1-(3-methoxypropylamino)ethyl, 1-(cyclopropylmethylamino)ethyl,
1-(dimethylphosphoryl-methyl-amino)ethyl, 1-acetamidoethyl, 1-cyano-1-methyl-ethyl,
1-dimethylaminoethyl, 1-piperidyl, 1-piperidylsulfonyl, 1-propylaminoethyl,
1-pyrrolidin-1-ylethyl, 2-(1-methylpyrrolidin-2-yl)ethylcarbamoyl, 2-(1-piperidyl)ethoxy,
2-(1-piperidyl)ethylcarbamoyl, 2-(2-hydroxyethoxy)ethylcarbamoyl,
2-(2-pyridyl)ethylcarbamoyl, 2-(isopropylamino)ethylcarbamoyl,
2-(tert-butoxycarbonylamino)ethylcarbamoyl, 2,3-dihydroxypropylcarbamoyl,
2-aminoethylcarbamoyl, 2-dimethylaminoethoxy, 2-dimethylaminoethylcarbamoyl,
2-hydroxyethyl, 2-hydroxyethylcarbamoyl, 2-methoxyethoxy, 2-methoxyethylcarbamoyl,
2-methoxyethyl-methyl-amino, 2-methoxyethylsulfamoyl, 2-methylaminoethylcarbamoyl,
2-morpholinoethoxy, 2-morpholinoethylcarbamoyl, 2-oxopyrrolidin-1-yl,
2-piperidylmethylcarbamoyl, 2-pyrrolidin-1-ylethoxy, 2-pyrrolidin-1-ylethylcarbamoyl,
3-(2-oxopyrrolidin-1-yl)propylcarbamoyl,
3-(methyl-tert-butoxycarbonyl-amino)propylcarbamoyl,
3-(tert-butoxycarbonylamino)propylcarbamoyl, 3-aminopropylcarbamoyl,
3-dimethylaminopropylcarbamoyl, 3-dimethylaminopyrrolidine-1-carbonyl,
3-hydroxybutylcarbamoyl, 3-imidazol-1-ylpropylcarbamoyl, 3-methoxypropanoylamino,
3-methoxypropanoyl-methyl-amino, 3-methylaminopropylcarbamoyl, methylsulfonyl,
3-morpholinopropylcarbamoyl, 4-acetylpiperazine-1-carbonyl,
4-methyl-1,4-diazepane-1-carbonyl, 4-methylpiperazine-1-carbonyl, 4-piperidylcarbamoyl,
4-piperidylmethylcarbamoyl, acetamido, acetyl, acetyl-methyl-amino, amino, butylsulfamoyl, carboxy, chloro, formyl, difluoromethylsulfonyl, dimethylamino, dimethylcarbamoyl, ethoxy, ethyl-(2-hydroxyethyl)amino, fluoro, hydroxy, hydroxymethyl, isopropoxy, methoxy, methoxycarbonyl, methyl, methylcarbamoyl, morpholino, morpholinosulfonyl, pyrazol-1-yl, pyrrolidin-1-ylsulfonyl, pyrrolidin-2-ylmethylcarbamoyl, tetrahydrofuran-2-ylmethylcarbamoyl, trifluoromethoxy and trifluoromethyl.

R¹ is a substituent on carbon and is selected from fluoro, chloro, hydroxy, methyl, methoxy, ethoxy, isopropoxy, mesyl, formyl, 1-cyano-1-methylethyl, ethoxycarbonyl, piperidin-4-ylaminocarbonyl, N,N-dimethylaminocarbamoyl, morpholino, acetylamino, N-methyl-N-acetylamino, N-ethyl-N-(2-hydroxyethyl)amino, 2-(pyrrolidin-1-yl)ethoxy, 2-(piperidin-1-yl)ethoxy, 2-(morpholino)ethoxy, 2-(dimethylamino)ethoxy, piperidin-1-yl, trifluoromethyl, N-butylsulphamoyl, morpholinosulphonyl, difluoromesyl, pyrrolidin-1-ylsulphonyl, trifluoromethoxy, piperidin-1-ylsulphonyl, hydroxymethyl, 2-morpholinoethylaminomethyl, 2-methoxyethylaminomethyl, N-methylcarbamoyl,
N-(tetrahydrofur-2-ylmethyl)carbamoyl,
N-[(2,2-dimethyl-1,3-dioxolan-4-yl)methyl]carbamoyl,
N-[(1-t-butoxycarbonylpiperidin-4-yl)methyl]carbamoyl,
N-[(1-t-butoxycarbonylpyrrolidin-2-yl)methyl]carbamoyl,
N-(pyrrolidin-2-ylmethyl)carbamoyl, N-(piperidin-2-ylmethyl)carbamoyl,
N-(piperidin-4-ylmethyl)carbamoyl, N-(2-methoxyethyl)carbamoyl,
N-(2-methoxy-1-methylethyl)carbamoyl, N-(2-piperidin-1-ylethyl)carbamoyl,
N-(2-pyrrolidin-1-ylethyl)carbamoyl, N-[2-(1-methylpyrrolidin-2-yl)ethyl]carbamoyl,
N-(2-dimethylaminoethyl)carbamoyl, N-(2-methylaminoethyl)carbamoyl,
N-(2-isopropylaminoethyl)carbamoyl, N-(2-morpholinoethyl)carbamoyl,
N-(2-aminoethyl)carbamoyl, N-(2-hydroxyethyl)carbamoyl, N-(2-pyrid-2-ylethyl)carbamoyl,
N-[2-(2-hydroxyethoxy)ethyl]carbamoyl, N-[2-(t-butoxycarbonylamino)ethyl]carbamoyl,
N-{3-[N-(t-butoxycarbonyl)-N-methylamino]propyl}carbamoyl,
N-(3-methylaminopropyl)carbamoyl, N-(3-dimethylaminopropyl)carbamoyl,
N-(2,3-dihydroxypropyl)carbamoyl, N-[3-(2-oxopyrrolidin-1-yl)propyl]carbamoyl,
N-(3-morpholinopropyl)carbamoyl, N-(3-aminopropyl)carbamoyl,
N-[3-(t-butoxycarbonylamino)propyl]carbamoyl, N-(3-imidazol-1-ylpropyl)carbamoyl and
N-(3-hydroxybutyl)carbamoyl.

R¹ is a substituent on carbon and is selected from N-(2-methoxyethyl)carbamoyl, 1-methyl-1-cyanoethyl and N-(2-morpholinoethyl)carbamoyl.

n is selected from 0-2; wherein the values of R¹ may be the same or different.

n is 2; wherein the values of R¹ may be the same or different.

n is 1.

n is 0.

Ring A, R¹ and n together form, 1-methylpyrazol-3-yl,
2,5-dioxabicyclo[4.4.0]deca-7,9,11-trien-8-yl,
2-methoxy-4-(2-methoxyethylcarbamoyl)phenyl, 2-methylbenzooxazol-5-yl,
3-(1-piperidylsulfonyl)phenyl, 3-(2-methoxyethylcarbamoyl)phenyl,
3-(butylsulfamoyl)phenyl, 3-(trifluoromethyl)phenyl,
3-chloro-4-(2-methoxyethylcarbamoyl)phenyl, 3-fluoro-4-(2-methoxyethylcarbamoyl)phenyl,
3-fluorophenyl, 3-isopropoxyphenyl, 3-methoxy-4-methyl-phenyl,
3-methoxy-5-(trifluoromethyl)phenyl, 3-methyl-4-(2-morpholinoethylcarbamoyl)phenyl,
3-methyl-4-[2-(1-piperidyl)ethylcarbamoyl]phenyl, 3-methylsulfonylphenyl,
3-morpholinophenyl, 3-morpholinosulfonylphenyl, 3-pyrrolidin-1-ylsulfonylphenyl,
4-(1-acetamidoethyl)phenyl, 4-(1-cyano-1-methyl-ethyl)phenyl,
4-(1-dimethylaminoethyl)phenyl, 4-(1-piperidyl)phenyl, 4-(1-propylaminoethyl)phenyl,
4-(1-pyrrolidin-1-ylethyl)phenyl, 4-(2,3-dihydroxypropylcarbamoyl)phenyl,
4-(2-aminoethylcarbamoyl)phenyl, 4-(2-dimethylaminoethoxy)phenyl,
4-(2-dimethylaminoethylcarbamoyl)-3-methyl-phenyl,
4-(2-dimethylaminoethylcarbamoyl)phenyl, 4-(2-hydroxyethyl)phenyl,
4-(2-hydroxyethylcarbamoyl)phenyl, 4-(2-methoxyethoxy)phenyl,
4-(2-methoxyethylcarbamoyl)-2-pyridyl, 4-(2-methoxyethylcarbamoyl)-3-methyl-phenyl,
4-(2-methoxyethylcarbamoyl)phenyl, 4-(2-methoxyethyl-methyl-amino)phenyl,
4-(2-methoxyethylsulfamoyl)phenyl, 4-(2-methylaminoethylcarbamoyl)-2-pyridyl,
4-(2-methylaminoethylcarbamoyl)phenyl, 4-(2-morpholinoethoxy)phenyl,
4-(2-morpholinoethylcarbamoyl)phenyl, 4-(2-oxopyrrolidin-1-yl)phenyl,
4-(2-piperidylmethylcarbamoyl)phenyl, 4-(2-pyrrolidin-1-ylethoxy)phenyl,
4-(2-pyrrolidin-1-ylethylcarbamoyl)phenyl, 4-(3-aminopropylcarbamoyl)phenyl,
4-(3-dimethylaminopropylcarbamoyl)phenyl,
4-(3-dimethylaminopyrrolidine-1-carbonyl)phenyl, 4-(3-hydroxybutylcarbamoyl)phenyl,
4-(3-imidazol-1-ylpropylcarbamoyl)phenyl, 4-(3-methoxypropanoylamino)phenyl,
4-(3-methoxypropanoyl-methyl-amino)phenyl, 4-(3-methylaminopropylcarbamoyl)phenyl,
4-(3-morpholinopropylcarbamoyl)phenyl, 4-(4-acetylpiperazine-1-carbonyl)phenyl,
4-(4-methyl-1,4-diazepane-1-carbonyl)phenyl, 4-(4-methylpiperazine-1-carbonyl)phenyl,
4-(4-piperidylcarbamoyl)phenyl, 4-(4-piperidylmethylcarbamoyl)phenyl,
4-(acetyl-methyl-amino)phenyl, 4-(difluoromethylsulfonyl)phenyl,
4-(dimethylcarbamoyl)-3-methyl-phenyl, 4-(dimethylcarbamoyl)phenyl,
4-(ethyl-(2-hydroxyethyl)amino)phenyl, 4-(hydroxymethyl)phenyl,
4-(methylcarbamoyl)phenyl, 4-(pyrrolidin-2-ylmethylcarbamoyl)phenyl,
4-(tetrahydrofuran-2-ylmethylcarbamoyl)phenyl, 4-(trifluoromethoxy)phenyl,
4-[(1R)-1-(3-methoxypropanoylamino)ethyl]phenyl, 4-[(1R)-1-acetamidoethyl]phenyl,
4-[(1R)-1-dimethylaminoethyl]phenyl, 4-[(1S)-1-(3-methoxypropanoylamino)ethyl]phenyl,
4-[(1S)-1-acetamidoethyl]phenyl, 4-[(1S)-1-dimethylaminoethyl]phenyl,
4-[(1-tert-butoxycarbonyl-4-piperidyl)methylcarbamoyl]phenyl,
4-[(1-tert-butoxycarbonylpyrrolidin-2-yl)methylcarbamoyl]phenyl,
4-[(2,2-dimethyl-1,3-dioxolan-4-yl)methylcarbamoyl]phenyl,
4-[(2-dimethylaminoethylamino)methyl]phenyl,
4-[(2-hydroxyethyl-methyl-amino)methyl]phenyl,
4-[(2-methoxy-1-methyl-ethyl)carbamoyl]phenyl, 4-[(2-methoxyethylamino)methyl]phenyl,
4-[(2-methoxyethyl-methyl-amino)methyl]phenyl,
4-[(2-morpholinoethylamino)methyl]phenyl,
4-[(3S)-3-dimethylaminopyrrolidine-1-carbonyl]phenyl,
4-[(4-methylpiperazin-1-yl)methyl]phenyl, 4-[(ethyl-(2-hydroxyethyl)amino)methyl]phenyl,
4-[[(2-dimethylamino-1-methyl-ethyl)amino]methyl]phenyl,
4-[[2-(1-methylpyrrolidin-2-yl)ethylamino]methyl]phenyl,
4-[1-(2-hydroxyethylamino)ethyl]phenyl, 4-[1-(2-hydroxyethyl-methyl-amino)ethyl]phenyl,
4-[1-(2-methoxyethylamino)ethyl]phenyl, 4-[1-(3-hydroxybutylamino)ethyl]phenyl,
4-[1-(3-hydroxypropylamino)ethyl]phenyl,
4-[1-(3-hydroxypropyl-methyl-amino)ethyl]phenyl,
4-[1-(3-methoxypropanoylamino)ethyl]phenyl, 4-[1-(3-methoxypropylamino)ethyl]phenyl,
4-[1-(cyclopropylmethylamino)ethyl]phenyl,
4-[1-(dimethylphosphoryl-methyl-amino)ethyl]-3-methyl-phenyl,
4-[2-(1-methylpyrrolidin-2-yl)ethylcarbamoyl]phenyl, 4-[2-(1-piperidyl)ethoxy]phenyl,
4-[2-(1-piperidyl)ethylcarbamoyl]phenyl, 4-[2-(2-hydroxyethoxy)ethylcarbamoyl]phenyl,
4-[2-(2-pyridyl)ethylcarbamoyl]phenyl, 4-[2-(isopropylamino)ethylcarbamoyl]phenyl,
4-[2-(tert-butoxycarbonylamino)ethylcarbamoyl]phenyl,
4-[3-(2-oxopyrrolidin-1-yl)propylcarbamoyl]phenyl,
4-[3-(methyl-tert-butoxycarbonyl-amino)propylcarbamoyl]phenyl,
4-[3-(tert-butoxycarbonylamino)propylcarbamoyl]phenyl, 4-acetamidophenyl,
4-acetylphenyl, 4-aminophenyl, 4-carboxyphenyl, 4-dimethylaminophenyl, 4-ethoxyphenyl,
4-fluorophenyl, 4-hydroxyphenyl, 4-methoxycarbonylphenyl, 4-methoxyphenyl,
4-methylsulfonylphenyl, 4-morpholinophenyl, 4-morpholinosulfonylphenyl,
4-pyrazol-1-ylphenyl, 4-pyrrolidin-1-ylsulfonylphenyl,
5-(2-methoxyethylcarbamoyl)-2-pyridyl, 6-(2-methoxyethylcarbamoyl)-2-pyridyl,
6-(2-methylaminoethylcarbamoyl)-2-pyridyl, 6-morpholino-3-pyridyl, benzo[1,3]dioxol-5-yl, m-tolyl or p-tolyl.

m is 0 or 1.

m is 1.

m is 0.

R² is selected from halo, C₁₋₆alkyl or C₁₋₆alkoxy.

R² is selected from C₁₋₆alkyl or C₁₋₆alkoxy.

R² is selected from fluoro, chloro, methyl or methoxy.

R² is selected from methyl or methoxy.

R² is methoxy.

R² is methyl.

Therefore in a further aspect of the invention there is provided a compound of formula (I) (as depicted above) wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, hydroxy, amino, carboxy, C₁₋₆alkyl, C₁₋₆alkoxy, C₁₋₆alkanoyl, N,N—(C₁₋₆alkyl)₂amino, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 2, C₁₋₆alkoxycarbonyl, N—(C₁₋₆alkyl)sulphamoyl or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

n is selected from 0-2; wherein the values of R¹ may be the same or different;

m is 0 or 1;

R² is selected from halo, C₁₋₆alkyl or C₁₋₆alkoxy;

R³ is selected from C₁₋₆alkyl;

R⁶ is selected from halo, cyano, hydroxy, amino, C₁₋₆alkoxy, N—(C₁₋₆alkyl)amino, N,N—(C₁₋₆alkyl)₂amino, C₁₋₆alkanoylamino, C₁₋₆alkoxycarbonylamino, N—(C₁₋₆alkyl)-N—(C₁₋₆alkoxycarbonyl)amino, (R³⁵)(R³⁶)P(O)N(C₁₋₆alkyl)- or heterocyclyl-R¹³—; wherein R⁶ may be optionally substituted on carbon by one or more R¹⁵; and wherein if said heterocyclyl contains an —NH— moiety that nitrogen may be optionally substituted by a group selected from R¹⁴;

R³⁵ and R³⁶ are independently selected from C₁₋₆alkyl;

R⁵ and R¹³ are independently selected from a direct bond, —C(O)—, —C(O)N(R¹⁸)— or —S(O)ₛ—; wherein R¹⁸ is hydrogen and s is 0-2;

R⁷ and R¹⁴ are independently selected from C₁₋₆alkyl, C₁₋₆alkanoyl and C₁₋₆alkoxycarbonyl; and

R¹⁵ is selected from hydroxy, methyl, methoxy, dimethylamino, carbocyclyl or heterocyclyl; wherein if said heterocyclyl contains an —NH— moiety that nitrogen may be optionally substituted by methyl;

or a pharmaceutically acceptable salt thereof.

Therefore in a further aspect of the invention there is provided a compound of formula (I) (as depicted above) wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, hydroxy, C₁₋₆alkyl, C₁₋₆alkoxy, N,N—(C₁₋₆alkyl)₂amino, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 2, C₁₋₆alkoxycarbonyl, N—(C₁₋₆alkyl)sulphamoyl or heterocyclyl-R⁵—; wherein R¹ may be optionally substituted on carbon by one or more R⁶;

n is selected from 0-2; wherein the values of R¹ may be the same or different;

R² is selected from C₁₋₆alkyl or C₁₋₆alkoxy;

m is 0 or 1;

R³ is selected from C₁₋₆alkyl;

R⁶ is selected from halo, cyano, hydroxy, amino, C₁₋₆alkoxy, N—(C₁₋₆alkyl)amino, N,N—(C₁₋₆alkyl)₂amino, C₁₋₆alkoxycarbonylamino, N—(C₁₋₆alkyl)-N—(C₁₋₆alkoxycarbonyl)amino or heterocyclyl-R¹³—; wherein R⁶ may be optionally substituted on carbon by one or more R¹⁵; and wherein if said heterocyclyl contains an —NH— moiety that nitrogen may be optionally substituted by a group selected from R¹⁴;

R⁵ and R¹³ are independently selected from a direct bond, —N(R¹⁷)C(O)— or —S(O)ₛ—; wherein R¹⁷ is hydrogen and s is 2;

R¹⁴ is selected from C₁₋₆alkyl or C₁₋₆alkoxycarbonyl; and

R¹⁵ is selected from hydroxy, methyl, methoxy or heterocyclyl;

or a pharmaceutically acceptable salt thereof.

Therefore in a further aspect of the invention there is provided a compound of formula (I) (as depicted above) wherein:

Ring A is carbocyclyl;

R¹ is a substituent on carbon and is selected from C₁₋₆alkyl or N—(C₁₋₆alkyl)carbamoyl; wherein R¹ may be optionally substituted on carbon by one or more R⁶;

n is 1;

R² is selected from C₁₋₆alkyl or C₁₋₆alkoxy;

m is 0 or 1;

R⁶ is selected from cyano, C₁₋₆alkoxy or heterocyclyl-R¹³—; and

R¹³ is selected from a direct bond;

or a pharmaceutically acceptable salt thereof.

Therefore in a further aspect of the invention there is provided a compound of formula (I) (as depicted above) wherein:

Ring A is phenyl, pyrid-2-yl, pyrid-3-yl, 1,3-benzodioxol-5-yl, 2,3-dihydro-1,4-benzodioxin-6-yl, benzooxazol-5-yl or 1-methylpyrazol-3-yl.

R¹ is a substituent on carbon and is selected from
(1R)-1-(3-methoxypropanoylamino)ethyl, (1R)-1-acetamidoethyl,
(1R)-1-dimethylaminoethyl, (1S)-1-(3-methoxypropanoylamino)ethyl, (1S)-1-acetamidoethyl,
(1S)-1-dimethylaminoethyl, (1-tert-butoxycarbonyl-4-piperidyl)methylcarbamoyl,
(1-tert-butoxycarbonylpyrrolidin-2-yl)methylcarbamoyl,
(2,2-dimethyl-1,3-dioxolan-4-yl)methylcarbamoyl, (2-dimethylaminoethylamino)methyl,
(2-hydroxyethyl-methyl-amino)methyl, (2-methoxy-1-methyl-ethyl)carbamoyl,
(2-methoxyethylamino)methyl, (2-methoxyethyl-methyl-amino)methyl,
(2-morpholinoethylamino)methyl, (3S)-3-dimethylaminopyrrolidine-1-carbonyl,
(4-methylpiperazin-1-yl)methyl, (ethyl-(2-hydroxyethyl)amino)methyl,
[(2-dimethylamino-1-methyl-ethyl)amino]methyl,
[2-(1-methylpyrrolidin-2-yl)ethylamino]methyl, 1-(2-hydroxyethylamino)ethyl,
1-(2-hydroxyethyl-methyl-amino)ethyl, 1-(2-methoxyethylamino)ethyl,
1-(3-hydroxybutylamino)ethyl, 1-(3-hydroxypropylamino)ethyl,
1-(3-hydroxypropyl-methyl-amino)ethyl, 1-(3-methoxypropanoylamino)ethyl,
1-(3-methoxypropylamino)ethyl, 1-(cyclopropylmethylamino)ethyl,
1-(dimethylphosphoryl-methyl-amino)ethyl, 1-acetamidoethyl, 1-cyano-1-methyl-ethyl,
1-dimethylaminoethyl, 1-piperidyl, 1-piperidylsulfonyl, 1-propylaminoethyl,
1-pyrrolidin-1-ylethyl, 2-(1-methylpyrrolidin-2-yl)ethylcarbamoyl, 2-(1-piperidyl)ethoxy,
2-(1-piperidyl)ethylcarbamoyl, 2-(2-hydroxyethoxy)ethylcarbamoyl,
2-(2-pyridyl)ethylcarbamoyl, 2-(isopropylamino)ethylcarbamoyl,
2-(tert-butoxycarbonylamino)ethylcarbamoyl, 2,3-dihydroxypropylcarbamoyl,
2-aminoethylcarbamoyl, 2-dimethylaminoethoxy, 2-dimethylaminoethylcarbamoyl,
2-hydroxyethyl, 2-hydroxyethylcarbamoyl, 2-methoxyethoxy, 2-methoxyethylcarbamoyl,
2-methoxyethyl-methyl-amino, 2-methoxyethylsulfamoyl, 2-methylaminoethylcarbamoyl,
2-morpholinoethoxy, 2-morpholinoethylcarbamoyl, 2-oxopyrrolidin-1-yl,
2-piperidylmethylcarbamoyl, 2-pyrrolidin-1-ylethoxy, 2-pyrrolidin-1-ylethylcarbamoyl,
3-(2-oxopyrrolidin-1-yl)propylcarbamoyl,
3-(methyl-tert-butoxycarbonyl-amino)propylcarbamoyl,
3-(tert-butoxycarbonylamino)propylcarbamoyl, 3-aminopropylcarbamoyl,
3-dimethylaminopropylcarbamoyl, 3-dimethylaminopyrrolidine-1-carbonyl,
3-hydroxybutylcarbamoyl, 3-imidazol-1-ylpropylcarbamoyl, 3-methoxypropanoylamino,
3-methoxypropanoyl-methyl-amino, 3-methylaminopropylcarbamoyl, methylsulfonyl,
3-morpholinopropylcarbamoyl, 4-acetylpiperazine-1-carbonyl,
4-methyl-1,4-diazepane-1-carbonyl, 4-methylpiperazine-1-carbonyl, 4-piperidylcarbamoyl,
4-piperidylmethylcarbamoyl, acetamido, acetyl, acetyl-methyl-amino, amino, butylsulfamoyl, carboxy, chloro, formyl, difluoromethylsulfonyl, dimethylamino, dimethylcarbamoyl, ethoxy, ethyl-(2-hydroxyethyl)amino, fluoro, hydroxy, hydroxymethyl, isopropoxy, methoxy, methoxycarbonyl, methyl, methylcarbamoyl, morpholino, morpholinosulfonyl, pyrazol-1-yl, pyrrolidin-1-ylsulfonyl, pyrrolidin-2-ylmethylcarbamoyl, tetrahydrofuran-2-ylmethylcarbamoyl, trifluoromethoxy and trifluoromethyl;

n is selected from 0-2; wherein the values of R¹ may be the same or different;

m is 0 or 1;

R² is selected from fluoro, chloro, methyl or methoxy;

or a pharmaceutically acceptable salt thereof.

Therefore in a further aspect of the invention there is provided a compound of formula (I) (as depicted above) wherein:

Ring A is phenyl, pyrid-2-yl, pyrid-3-yl, 1,3-benzodioxol-5-yl, 2,3-dihydro-1,4-benzodioxin-6-yl or 1-methylpyrazol-3-yl;

R¹ is a substituent on carbon and is selected from fluoro, chloro, hydroxy, methyl, methoxy, ethoxy, isopropoxy, mesyl, formyl, 1-cyano-1-methylethyl, ethoxycarbonyl, piperidin-4-ylaminocarbonyl, N,N-dimethylaminocarbamoyl, morpholino, acetylamino, N-methyl-N-acetylamino, N-ethyl-N-(2-hydroxyethyl)amino, 2-(pyrrolidin-1-yl)ethoxy, 2-(piperidin-1-yl)ethoxy, 2-(morpholino)ethoxy, 2-(dimethylamino)ethoxy, piperidin-1-yl, trifluoromethyl, N-butylsulphamoyl, morpholinosulphonyl, difluoromesyl, pyrrolidin-1-ylsulphonyl, trifluoromethoxy, piperidin-1-ylsulphonyl, hydroxymethyl, 2-morpholinoethylaminomethyl, 2-methoxyethylaminomethyl, N-methylcarbamoyl,
N-(tetrahydrofur-2-ylmethyl)carbamoyl,
N-[(2,2-dimethyl-1,3-dioxolan-4-yl)methyl]carbamoyl,
N-[(1-t-butoxycarbonylpiperidin-4-yl)methyl]carbamoyl,
N-[(1-t-butoxycarbonylpyrrolidin-2-yl)methyl]carbamoyl,
N-(pyrrolidin-2-ylmethyl)carbamoyl, N-(piperidin-2-ylmethyl)carbamoyl,
N-(piperidin-4-ylmethyl)carbamoyl, N-(2-methoxyethyl)carbamoyl,
N-(2-methoxy-1-methylethyl)carbamoyl, N-(2-piperidin-1-ylethyl)carbamoyl,
N-(2-pyrrolidin-1-ylethyl)carbamoyl, N-[2-(1-methylpyrrolidin-2-yl)ethyl]carbamoyl,
N-(2-dimethylaminoethyl)carbamoyl, N-(2-methylaminoethyl)carbamoyl,
N-(2-isopropylaminoethyl)carbamoyl, N-(2-morpholinoethyl)carbamoyl,
N-(2-aminoethyl)carbamoyl, N-(2-hydroxyethyl)carbamoyl, N-(2-pyrid-2-ylethyl)carbamoyl,
N-[2-(2-hydroxyethoxy)ethyl]carbamoyl, N-[2-(t-butoxycarbonylamino)ethyl]carbamoyl,
N-{3-[N-(t-butoxycarbonyl)-N-methylamino]propyl}carbamoyl,
N-(3-methylaminopropyl)carbamoyl, N-(3-dimethylaminopropyl)carbamoyl,
N-(2,3-dihydroxypropyl)carbamoyl, N-[3-(2-oxopyrrolidin-1-yl)propyl]carbamoyl,
N-(3-morpholinopropyl)carbamoyl, N-(3-aminopropyl)carbamoyl,
N-[3-(t-butoxycarbonylamino)propyl]carbamoyl, N-(3-imidazol-1-ylpropyl)carbamoyl and
N-(3-hydroxybutyl)carbamoyl;

n is selected from 0-2; wherein the values of R¹ may be the same or different;

m is 0 or 1;

R² is selected from methyl or methoxy;

or a pharmaceutically acceptable salt thereof.

Therefore in a further aspect of the invention there is provided a compound of formula (I) (as depicted above) wherein:

Ring A is phenyl;

R¹ is a substituent on carbon and is selected from N-(2-methoxyethyl)carbamoyl, 1-methyl-1-cyanoethyl and N-(2-morpholinoethyl)carbamoyl;

n is 1;

m is 0 or 1; and

R² is selected from methyl or methoxy;

or a pharmaceutically acceptable salt thereof.

In another aspect of the invention, preferred compounds of the invention are any one of the Examples or a pharmaceutically acceptable salt thereof.

In another aspect of the invention, a particular compound of the invention is N-{4-[(1S)-1-(propylamino)ethyl]phenyl}-6-pyridin-4-ylquinazolin-2-amine, N-{4-[(1R)-1-(propylamino)ethyl]phenyl}-6-pyridin-4-ylquinazolin-2-amine, N-{4-[(R)-1-(dimethylamino)ethyl]phenyl}-6-pyridin-4-ylquinazolin-2-amine or N-{4-[(S)-1-(dimethylamino)ethyl]phenyl}-6-pyridin-4-ylquinazolin-2-amine or a pharmaceutically acceptable salt thereof.

In another aspect of the invention, particular compounds of the invention are any one of Examples 1, 49, 91, 124, 125, 126, 128, 129, 131, 136, 139 or 140 or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)), comprises of:

Process a) reacting an amine of formula (II):

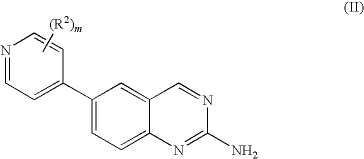

with a compound of formula (III):

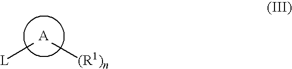

wherein L is a displaceable atom or group; or
Process b) reacting a compound of formula (IV):

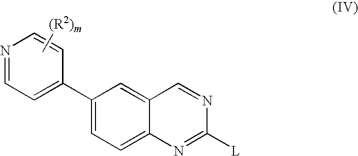

wherein L is a displaceable atom or group; with an amine of formula (V):

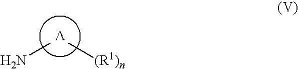

or
Process c) reacting a compound of formula (VI):

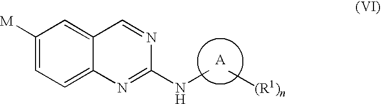

wherein M is an organometallic or organoboron reagent; with a compound of formula (VII):

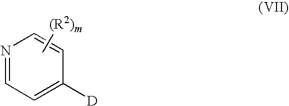

wherein D is a displaceable atom or group; or
Process d) reacting a compound of formula (VIII):

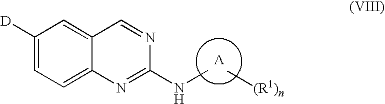

wherein D is a displaceable atom or group; with a compound of formula (IX):

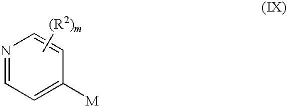

wherein M is an organometallic or organoboron reagent;
and thereafter if necessary:
i) converting a compound of the formula (I) into another compound of the formula (I);
ii) removing any protecting groups;
iii) forming a pharmaceutically acceptable salt.

L is a displaceable atom or group, suitable values for L are for example, a halo or sulphonyloxy group, for example a chloro, bromo, methanesulphonyloxy or toluene-4-sulphonyloxy group.

D is a displaceable atom or group, suitable values for L include chloro, bromo, tosyl and trifluoromethylsulphonyloxy.

M is an organometallic or organoboron reagent, suitable values for M include organoboron and organotin reagents, in particular B(OR^{z})₂ where R^{z} is hydrogen or C₁₋₆alkyl for example B(OH)₂; and Sn(R^{y})₃ where R^{y} is C₁₋₆alkyl for example Sn(Bu)₃.

Specific reaction conditions for the above reactions are as follows.

Processes a) and b) Compounds of formula (II) and (III) and compounds of formula (IV) and (V) can be reacted together by coupling chemistry utilizing an appropriate catalyst and ligand such as Pd₂(dba)₃ and BINAP respectively and a suitable base such as sodium tert-butoxide or caesium carbonate. The reaction usually requires thermal conditions often in the range of 80° C. to 100° C.

Amines of formula (II) may be prepared according to Scheme 1:

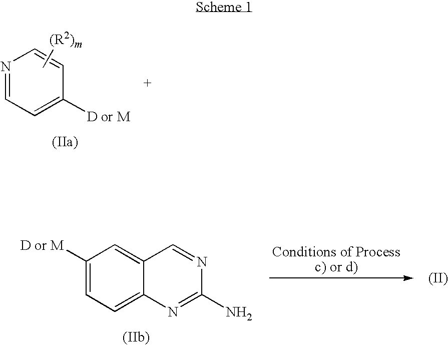

wherein the NH₂ group would optionally need protecting.

Compounds of formula (IV) may be prepared according to Scheme 1) wherein the NH₂ group is an L group.

Compounds of formula (IIa), (IIb), (III) and (V) are commercially available compounds, or they are known in the literature or they may be prepared by standard processes known in the art.

Processes c) and d) Compounds of formula (VI) and (VII), and (VIII) and (IX) may be reacted together by coupling chemistry utilizing an appropriate catalyst. Such reactions are well known in the art. For example, where M is an organoboron group, Pd(PPh₃)₄ and a suitable base such as sodium carbonate or caesium carbonate can be utilized. In the case where M is an organotin reagent, Pd(PPh₃)₄ can be utilized as the catalyst. The reactions take place in suitable solvents and may require thermal conditions.

Compounds of formula (VI) may be prepared according to Scheme 2:

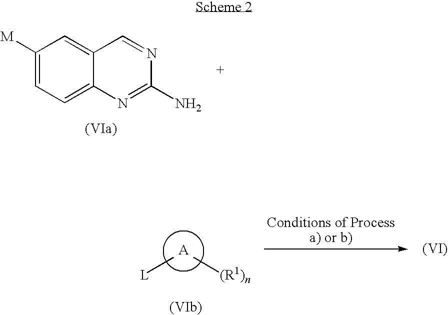

wherein L is a displaceable atom or group as defined herein above.

Compounds of formula (VIII) may be prepared according to Scheme 2) wherein the M group is a D group.

Compounds of formula (VIa), (VIb), (VII) and (IX) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below.

### Biological Activity

### B-Raf Alpha Screen Assay

Activity of purified full length His-tagged Mutant B-Raf (V600E) enzyme (MT B-Raf) may be determined in vitro using an Amplified Luminescent Proximity Homogeneous Assay (ALPHA) (Perkin Elmer, MA), which measures phosphorylation of the MT B-Raf substrate, biotinylated HIS-MEK-AVI (PLAZA internal database, construct #pAZB0141), as described below. MT B-Raf may be expressed in insect cells and affinity purified by Ni⁺² agarose followed by Q-Sepharose chromatography. Typical yields can be 1.08 mg/ml at >90% purity.

The phosphorylation of the MT B-Raf substrate in the presence and absence of the compound of interest may be determined. Briefly, 5 μl of enzyme/substrate/adenosine triphosphate (ATP) mix consisting of 0.12 nM MT B-Raf, 84 nM biotinylated HIS-MEK-AVI, and 24 μM ATP in 1.2× buffer may be preincubated with 2 μl of compound for 20 minutes at 25° C. Reactions can be initiated with 5 μl of Metal mix consisting of 24 mM MgCl₂ in 1.2× buffer and incubated at 25° C. for 60 minutes and reactions can be stopped by addition of 5 μl of Detection mix consisting of 20 mM HEPES, 102 mM ethylenediamine tetraacetic acid, 1.65 mg/ml BSA, 136 mM NaCl, 3.4 nM Phospho-MEK1/2 (Ser217/221) antibody (Catalog #9121, Cell Signaling Technology, MA), 40 μg/ml Streptavidin donor beads (Perkin Elmer, MA, Catalog #6760002), and 40 μg/ml Protein A acceptor beads (Perkin Elmer, MA, Catalog #6760137). Plates may be incubated at 25° C. for 18 hours in the dark. Phosphorylated substrate can be detected by an EnVision plate reader (Perkin Elmer, MA) 680 nm excitation, 520-620 nm emission. Data can be graphed and IC₅₀s calculated using Excel Fit (Microsoft).

When tested in the above in vitro B-Raf Alpha screen assay, the compounds of the present invention exhibited activity less than 30 μM. For example the following result was obtained in a B-Raf Alpha screen comparable to the above wherein results quoted may be an average of two or more results:

| Ex No | IC50 (μM) |
|---|---|
| 1 | 0.077 |
| 2 | 0.73 |
| 3 | 0.27 |
| 4 | 0.4 |
| 5 | 0.18 |
| 6 | 0.33 |
| 7 | 0.14 |
| 8 | 0.12 |
| 9 | 0.23 |
| 10 | 0.35 |
| 11 | 0.34 |
| 12 | 0.12 |
| 13 | 0.31 |
| 14 | 0.21 |
| 15 | 0.089 |
| 16 | 0.2 |
| 17 | 0.079 |
| 18 | 1.3 |
| 19 | 0.49 |
| 20 | 0.25 |
| 21 | 0.2 |
| 22 | 0.091 |
| 23 | 0.13 |
| 24 | 0.52 |
| 25 | 0.39 |
| 26 | 0.55 |
| 27 | 0.087 |
| 28 | 0.14 |
| 29 | 0.053 |
| 30 | 0.083 |
| 31 | 0.082 |
| 32 | 0.13 |
| 33 | 0.16 |
| 34 | 0.27 |
| 35 | 0.11 |
| 36 | 0.095 |
| 37 | 1.7 |
| 38 | 1.5 |
| 39 | 0.1 |
| 40 | 0.16 |
| 41 | 0.46 |
| 42 | 0.23 |
| 43 | 0.16 |
| 44 | 0.42 |
| 45 | 0.3 |
| 46 | 0.13 |
| 47 | 0.37 |
| 48 | 0.27 |
| 49 | 0.038 |
| 50 | 0.15 |
| 51 | 0.13 |
| 52 | 0.12 |
| 53 | 0.21 |
| 54 | 0.12 |
| 55 | 0.022 |
| 56 | 0.026 |
| 57 | 0.029 |
| 58 | 0.036 |
| 59 | 0.069 |
| 60 | 0.15 |
| 61 | 0.1 |
| 62 | 0.093 |
| 63 | 0.098 |
| 64 | 0.022 |
| 65 | 1 |
| 66 | 0.7 |
| 67 | 0.11 |
| 68 | 0.11 |
| 69 | 0.041 |
| 70 | 0.074 |
| 71 | 0.19 |
| 72 | 0.26 |
| 73 | 0.3 |
| 74 | 0.16 |
| 75 | 0.024 |
| 76 | 0.029 |
| 77 | 0.099 |
| 78 | 0.066 |
| 79 | 0.13 |
| 80 | 0.067 |
| 81 | 0.061 |
| 82 | 3.1 |
| 83 | 0.63 |
| 84 | 0.82 |
| 85 | 0.39 |
| 86 | 1.5 |
| 87 | 2.2 |
| 88 | 1.82 |
| 89 | 0.28 |
| 90 | 0.11 |
| 91 | 0.057 |
| 92 | 0.092 |
| 93 | 3 |
| 94 | 0.3 |
| 95 | 1 |
| 96 | 0.74 |
| 97 | 0.45 |
| 98 | 0.63 |
| 99 | 1.1 |
| 100 | 1 |
| 101 | 1.7 |
| 102 | 0.52 |
| 103 | 0.062 |
| 104 | 0.16 |
| 105 | 1.2 |
| 106 | 23 |
| 107 | 1.9 |
| 108 | 0.25 |
| 109 | 0.94 |
| 110 | 0.15 |
| 111 | 0.17 |
| 112 | 0.21 |
| 113 | 0.1 |
| 114 | 6.8 |
| 115 | 0.52 |
| 116 | 0.063 |
| 117 | 0.073 |
| 118 | 0.093 |
| 119 | 0.064 |
| 120 | 0.056 |
| 122 | 0.078 |
| 123 | 0.041 |
| 124 | 0.027 |
| 125 | 0.032 |
| 126 | 0.051 |
| 127 | 0.036 |
| 128 | 0.067 |
| 129 | 0.019 |
| 130 | 0.12 |
| 131 | 0.019 |
| 132 | 0.073 |
| 133 | 0.046 |
| 134 | 0.035 |
| 135 | 0.029 |
| 136 | 0.037 |
| 137 | 0.08 |
| 138 | 0.047 |
| 139 | 0.036 |
| 140 | 0.021 |
| 141 | 0.034 |
| 142 | 0.012 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumours, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Thus according to this aspect of the invention there is provided a compound of the formula (I), or a pharmaceutically acceptable salt thereof, as defined hereinbefore for use as a medicament.

According to a further aspect of the invention there is provided t a compound of the formula (I), or a pharmaceutically acceptable salt thereof, as defined hereinbefore in the manufacture of a medicament for use in the production of a B-Raf inhibitory effect in a warm-blooded animal such as man.

According to this aspect of the invention there is provided a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided a compound of the formula (I), or a pharmaceutically acceptable salt thereof, as defined hereinbefore for use in the production of a B-Raf inhibitory effect in a warm-blooded animal such as man.

According to this aspect of the invention there is provided a compound of the formula (I), or a pharmaceutically acceptable salt thereof, as defined hereinbefore for use in the production of an anti-cancer effect in a warm-blooded animal such as man.

According to a further feature of the invention, there is provided a compound of the formula (I), or a pharmaceutically acceptable salt thereof, as defined herein before for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB 1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (Sutent™; WO 01/60814), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.
(x) cell cycle inhibitors including for example CDK inhibitors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C. unless otherwise stated;
(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz or 300 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are generally the millimolar ratio equivalents to those used in the previous example;
(xi) the following abbreviations have been used:
DMF N,N-dimethylformamide;
EtOAc ethyl acetate;
Pd₂(dba)₃ tris(dibenzylideneacetone)dipalladium (0);
BINAP (+/−)-2,2′-bis(diphenylphosphino)-1,1′-binaphthyl;
PdCl₂(dppf).CH₂Cl₂ dichloro[1,1′-bis(diphenylphosphino)ferrocene]palladium (II) dichloromethane adduct;
XANTPHOS 9,9-dimethyl-4,5-bis(diphenyl-phosphino)xanthene;
Pd(Ph₃P)₄ tetrakis(triphenylphosphine)palladium(0);
TFA trifluoroacetic acid;
DMSO dimethylsulphoxide;
DIPEA N,N,-diisopropylethylamine;
HATU N,N,N′,N′-tetramethyl-O-(7-azabenzotriazol-1-yl)uranium; hexafluorophosphate;
DME 1,2-dimethoxyethane;
DMA N,N,-dimethylacetamide;
DCM dichloromethane;
THF tetrahydrofuran;
MeOH methanol; and
Et₃N triethylamine;
   (xii) “ISCO” refers to normal phase flash column chromatography using pre-packed silica gel cartridges used according to the manufacturers instruction obtained from ISCO, Inc, 4700 Superior Street, Lincoln, Nebr., USA;
   (xiii) “Gilson HPLC” refers to a YMC-AQC18 reverse phase HPLC Column with dimension 20 mm/100 and 50 mm/250 in water-CH₃CN with 0.1% TFA, 10 mM ammonium acetate, formic acid, or ammonium hydroxide as mobile phase, obtained from Waters Corporation 34, Maple Street, Milford, Mass., USA; and
   (xiv) “Microwave” refers to a CEM Explorer® series microwave purchased from CEM Corporation, P.O. Box 200, 3100 Smith Farm Rd., Matthews, N.C., 28106, (704)-821-7015.

### Example 1

### N-(2-Methoxyethyl)-4-[(6-pyridin-4-ylquinazolin-2-yl)amino]benzamide

6-Pyridin-4-ylquinazolin-2-amine (Method 19; 50 mg, 0.225 mmol), 4-bromo-N-(2-methoxyethyl)benzamide (Method 3; 58 mg, 0.225 mmol), Cs₂CO₃ (220 mg, 0.675 mmol, 3.0 equiv) and BINAP (14 mg, 0.023 mmol, 10 mol %) in dioxane (2 ml) were treated with Pd₂(dba)₃ (11 mg, 0.012 mmol, 5 mol %). The reaction mixture was heated to 100° C. for 12 h. The reaction was then quenched with 10% NaOH (aq) and extracted with EtOAc. The organics were dried with NaCl(sat) and then Na₂SO₄(s). The organics were removed under reduced pressure and the resulting solid was purified by a Gilson HPLC (0.1% TFA in CH₃CN and water) to give 60 mg (52%) of the desired product. NMR: 10.38 (s, 1H), 9.45 (s, 1H), 8.86 (d, 2H), 8.61 (d, 1H), 8.38 (m, 2H), 8.22 (d, 2H), 8.07 (d, 2H), 7.87 (m, 3H), 3.44 (m, 4H), 3.27 (s, 3H); m/z 400.

### Examples 2-58

The following compounds were prepared by the procedure of Example 1, using the indicated starting materials.

| Ex | Compound | NMR | m/z | SM |
|---|---|---|---|---|
| 2 | 2-Methyl-2-{4-[(6- | 10.23 (s, 1H), 9.42 (s, 1H), | 366 | Method 2 and |
| | pyridin-4-ylquinazolin- | 8.89 (d, 2H), 8.62 (d, 1H), 8.39 (dd, | | Method 19 |
| | 2-yl)amino]phenyl}propanenitrile | 1H), 8.28 (d, 2H), 8.03 (d, 2H), | | |
| | | 7.82 (d, 1H), 7.49 (d, 2H), | | |
| | | 1.69 (s, 6H) | | |
| 3 | N-(2-Methoxyethyl)-4- | 10.27 (s, 1H), 9.41 (s, 1H), | 430 | Method 3 and |
| | {[6-(3-methoxypyridin- | 8.52 (s, 1H), 8.38 (m, 1H), 8.33 (d, | | Method 20 |
| | 4-yl)quinazolin-2- | 1H), 8.19 (m, 1H), 8.07 (d, 3H), | | |
| | yl]amino}benzamide | 7.85 (d, 2H), 7.78 (d, 1H), | | |
| | | 7.49 (d, 1H), 3.94 (s, 3H), 3.44 (m, | | |
| | | 4H), 3.27 (s, 3H) | | |
| 4 | N-(2-Methoxyethyl)-4- | 10.30 (s, 1H), 9.42 (s, 1H), | 414 | Method 3 and |
| | {[6-(2-methylpyridin- | 8.54 (d, 1H), 8.44 (s, 1H), 8.38 (m, | | Method 21 |
| | 4-yl)quinazolin-2- | 1H), 8.27 (d, 1H), 8.08 (m, 2H), | | |
| | yl]amino}benzamide | 7.84 (m, 2H), 7.72 (s, 1H), | | |
| | | 7.62 (m, 2H), 3.44 (m, 4H), 3.27 (s, | | |
| | | 3H), 2.56 (s, 3H) | | |
| 5 | N-(2-Morpholin-4- | 10.39 (s, 1H), 9.46 (s, 1H), | 455 | Method 19 and |
| | ylethyl)-4-[(6-pyridin- | 8.72 (m, 2H), 8.62 (m, 1H), 8.49 (s, | | Method 26 |
| | 4-ylquinazolin-2- | 1H), 8.31 (d, 1H), 8.12 (d, 2H), | | |
| | yl)amino]benzamide | 7.86 (m, 5H), 4.01 (m, 2H), | | |
| | | 3.65 (m, 7H), 3.17 (m, 3H) | | |
| 6 | N-(2-Methoxyethyl)-3- | 10.17 (s, 1H), 9.41 (s, 1H), | 400 | Method 19 and |
| | [(6-pyridin-4- | 8.68 (d, 2H), 8.45 (m, 3H), 8.29 (d, | | Method 25 |
| | ylquinazolin-2- | 1H), 8.16 (d, 1H), 7.84 (d, 2H), | | |
| | yl)amino]benzamide | 7.78 (d, 1H), 7.44 (m, 2H), | | |
| | | 3.58 (m, 4H), 3.29 (s, 3H) | | |
| 7 | N-(2-Hydroxyethyl)-4- | 10.31 (s, 1H), 9.43 (s, 1H), | 386 | Method 4 and |
| | [(6-pyridin-4- | 8.70 (m, 2H), 8.47 (s, 1H), 8.30 (d, | | Method 19 |
| | ylquinazolin-2- | 2H), 8.08 (d, 2H), 7.84 (m, 5H), | | |
| | yl)amino]benzamide | 4.74 (t, 1H), 3.50 (m, 2H), | | |
| | | 3.31 (m, 2H) | | |
| 8 | N-[2-(2- | 10.32 (s, 1H), 9.43 (s, 1H), | 430 | Method 5 and |
| | Hydroxyethoxy)ethyl]- | 8.69 (d, 2H), 8.47 (m, 1H), 8.31 (m, | | Method 19 |
| | 4-[(6-pyridin-4- | 1H), 8.29 (d, 1H), 8.08 (d, 2H), | | |
| | ylquinazolin-2- | 7.85 (m, 5H), 3.46 (m, 9H) | | |
| | yl)amino]benzamide | | | |
| 9 | Methyl 4-[(6-pyridin-4- | 10.50 (s, 1H), 9.46 (s, 1H), | 357 | Method 19 and |
| | ylquinazolin-2- | 8.69 (d, 2H), 8.48 (s, 1H), 8.31 (d, | | methyl 4- |
| | yl)amino]benzoate | 1H), 8.16 (d, 2H), 7.96 (d, 2H), | | bromobenzoate |
| | | 7.85 (m, 3H), 3.82 (s, 3H) | | |
| 10 | 2-Chloro-N-(2- | 10.39 (s, 1H), 9.45 (s, 1H), | 434 | Method 19 and |
| | methoxyethyl)-4-[(6- | 8.69 (d, 2H), 8.48 (s, 1H), 8.37 (m, | | Method 27 |
| | pyridin-4-ylquinazolin- | 1H), 8.31 (d, 1H), 8.22 (s, 1H), | | |
| | 2-yl)amino]benzamide | 7.96 (d, 1H), 7.85 (m, 3H), | | |
| | | 7.41 (d, 1H), 3.45 (m, 4H), 3.28 (s, | | |
| | | 3H) | | |
| 11 | 2-Fluoro-N-(2- | 10.52 (s, 1H), 9.47 (s, 1H), | 418 | Method 19 and |
| | methoxyethyl)-4-[(6- | 8.69 (d, 2H), 8.48 (s, 1H), 8.33 (d, | | Method 28 |
| | pyridin-4-ylquinazolin- | 1H), 8.16 (d, 1H), 8.02 (m, 1H), | | |
| | 2-yl)amino]benzamide | 7.86 (m, 3H), 7.71 (m, 2H), | | |
| | | 3.44 (m, 4H), 3.27 (s, 3H) | | |
| 12 | N-(2-Methoxyethyl)-2- | 10.12 (s, 1H), 9.41 (s, 1H), | 414 | Method 19 and |
| | methyl-4-[(6-pyridin-4- | 8.68 (d, 2H), 8.44 (m, 1H), 8.28 (d, | | Method 29 |
| | ylquinazolin-2- | 1H), 8.16 (m, 1H), 7.92 (d, 1H), | | |
| | yl)amino]benzamide | 7.83 (m, 4H), 7.33 (d, 1H), | | |
| | | 3.45 (m, 4H), 3.28 (s, 3H), 2.38 (s, | | |
| | | 3H) | | |
| 13 | tert-Butyl methyl[3- | 10.31 (s, 1H), 9.43 (s, 1H), | 513 | Method 19 and |
| | ({4-[(6-pyridin-4- | 8.69 (d, 2H), 8.46 (s, 1H), 8.30 (d, | | Method 30 |
| | ylquinazolin-2-yl)amino]benzoyl}amino)propyl] | 2H), 8.08 (d, 2H), 7.84 (m, 5H), | | |
| | carbamate | 3.21 (m, 4H), 2.78 (s, 3H), | | |
| | | 1.71 (m, 2H), 1.36 (br s, 9H) | | |
| 14 | 4-[(6-Pyridin-4- | 10.31 (s, 1H), 9.43 (s, 1H), | 426 | Method 19 and |
| | ylquinazolin-2- | 8.68 (d, 2H), 8.46 (m, 1H), 8.39 (m, | | Method 32 |
| | yl)amino]-N- | 1H), 8.30 (m, 1H), 8.07 (m, 2H), | | |
| | (tetrahydrofuran-2- | 7.85 (m, 5H), 3.98 (m, 1H), | | |
| | ylmethyl)benzamide | 3.78 (m, 1H), 3.62 (m, 1H), 3.29 (m, | | |
| | | 2H), 1.84 (m, 3H), 1.58 (m, 1H) | | |
| 15 | N-[2-(Dimethylamino)ethyl]- | 10.31 (s, 1H), 9.43 (s, 1H), | 413 | Method 6 and |
| | 4-[(6-pyridin-4- | 8.68 (d, 2H), 8.46 (m, 1H), 8.28 (m, | | Method 19 |
| | ylquinazolin-2- | 2H), 8.07 (m, 2H), 7.84 (m, 5H), | | |
| | yl)amino]benzamide | 3.36 (m, 2H), 2.39 (m, 2H), | | |
| | | 2.17 (s, 6H) | | |
| 16 | N-[(2,2-Dimethyl-1,3- | 10.32 (s, 1H), 9.43 (s, 1H), | 456 | Method 19 and |
| | dioxolan-4-yl)methyl]- | 8.69 (m, 2H), 8.46 (m, 2H), 8.29 (m, | | Method 33 |
| | 4-[(6-pyridin-4- | 1H), 8.08 (m, 2H), 7.85 (m, 5H), | | |
| | ylquinazolin-2- | 4.20 (m, 1H), 3.98 (m, 1H), | | |
| | yl)amino]benzamide | 3.71 (m, 1H), 3.48 (m, 2H), 1.35 (s, | | |
| | | 3H), 1.26 (s, 3H) | | |
| 17 | N-Methyl-4-[(6- | 10.31 (s, 1H), 9.43 (s, 1H), | 356 | Method 19 and |
| | pyridin-4-ylquinazolin- | 8.68 (d, 2H), 8.46 (m, 1H), 8.28 (m, | | Method 34 |
| | 2-yl)amino]benzamide | 2H), 8.05 (m, 2H), 7.84 (m, 5H), | | |
| | | 2.78 (d, 3H) | | |
| 18 | N-(2-Methoxyethyl)-6- | 10.60 (s, 1H), 9.50 (s, 1H), | 401 | Method 19 and |
| | [(6-pyridin-4- | 8.80 (m, 1H), 8.70 (m, 2H), 8.60 (m, | | Method 35 |
| | ylquinazolin-2- | 1H), 8.52 (m, 1H), 8.33 (m, 1H), | | |
| | yl)amino]nicotinamide | 8.28 (m, 1H), 7.90 (m, 3H), | | |
| | | 6.60 (m, 1H), 3.47 (m, 2H), 3.28 (s, | | |
| | | 3H), 3.16 (m, 2H) | | |
| 19 | tert-Butyl 2-[({4-[(6- | 10.31 (s, 1H), 9.43 (s, 1H), | 525 | Method 19 and |
| | pyridin-4-ylquinazolin- | 8.69 (d, 2H), 8.46 (s, 1H), 8.32 (br s, | | Method 36 |
| | 2-yl)amino]benzoyl}amino)methyl]pyrrolidine- | 1H), 8.28 (d, 1H), 8.08 (d, 2H), | | |
| | 1- | 7.85 (m, 5H), 3.92 (m, 1H), | | |
| | carboxylate | 3.25 (m, 4H), 1.80 (m, 4H), 1.40 (s, | | |
| | | 9H) | | |
| 20 | N-(2-Pyridin-2- | 10.32 (s, 1H), 9.44 (s, 1H), | 447 | Method 8 and |
| | ylethyl)-4-[(6-pyridin- | 8.69 (m, 2H), 8.55 (m, 1H), 8.47 (m, | | Method 19 |
| | 4-ylquinazolin-2- | 2H), 8.29 (m, 1H), 8.07 (d, 2H), | | |
| | yl)amino]benzamide | 7.83 (m, 6H), 7.32 (m, 2H), | | |
| | | 3.61 (m, 2H), 3.02 (m, 2H) | | |
| 21 | N-[3-(2-Oxopyrrolidin- | 10.34 (s, 1H), 9.44 (s, 1H), | 467 | Method 11 and |
| | 1-yl)propyl]-4-[(6- | 8.75 (m, 2H), 8.51 (m, 1H), 8.31 (m, | | Method 19 |
| | pyridin-4-ylquinazolin- | 2H), 8.08 (d, 2H), 7.98 (m, 2H), | | |
| | 2-yl)amino]benzamide | 7.84 (m, 3H), 3.23 (m, 6H), | | |
| | | 2.22 (m, 2H), 1.92 (m, 2H), 1.70 (m, | | |
| | | 2H) | | |
| 22 | N-(3-Morpholin-4- | 10.30 (s, 1H), 9.43 (s, 1H), | 469 | Method 19 and |
| | ylpropyl)-4-[(6- | 8.69 (d, 2H), 8.46 (d, 1H), 8.31 (m, | | Method 38 |
| | pyridin-4-ylquinazolin- | 2H), 8.07 (d, 2H), 7.84 (m, 5H), | | |
| | 2-yl)amino]benzamide | 3.56 (m, 4H), 3.27 (m, 2H), | | |
| | | 2.33 (m, 6H), 1.68 (m, 2H) | | |
| 23 | 4-[(6-Pyridin-4- | 10.31 (s, 1H), 9.43 (s, 1H), | 439 | Method 19 and |
| | ylquinazolin-2- | 8.69 (d, 2H), 8.46 (s, 1H), 8.28 (m, | | Method 39 |
| | yl)amino]-N-(2- | 2H), 8.07 (d, 2H), 7.84 (m, 5H), | | |
| | pyrrolidin-1- | 3.35 (m, 2H), 2.41-2.53 (m, 4H), | | |
| | ylethyl)benzamide | 1.67 (m, 6H) | | |
| 24 | tert-Butyl 4-[({4-[(6- | 10.31 (s, 1H), 9.43 (s, 1H), | 539 | Method 19 and |
| | pyridin-4-ylquinazolin- | 8.69 (d, 2H), 8.47 (s, 1H), 8.36 (m, | | Method 40 |
| | 2-yl)amino]benzoyl}amino)methyl]piperidine- | 1H), 8.30 (m, 1H), 8.08 (d, 2H), | | |
| | 1- | 7.86 (m, 5H), 3.92 (m, 2H), | | |
| | carboxylate | 3.15 (m, 2H), 2.72 (m, 2H), 1.68 (m, | | |
| | | 2H), 1.38 (s, 9H), 1.03 (3H) | | |
| 25 | tert-Butyl [2-({4-[(6- | 10.31 (s, 1H), 9.43 (s, 1H), | 485 | Method 19 and |
| | pyridin-4-ylquinazolin- | 8.69 (d, 2H), 8.46 (s, 1H), 8.32 (d, | | Method 41 |
| | 2-yl)amino]benzoyl}amino)ethyl]carbamate | 2H), 8.08 (d, 2H), 7.85 (m, 5H), | | |
| | | 6.94 (m, 1H), 3.29 (m, 2H), | | |
| | | 3.09 (m, 2H), 1.37 (s, 9H) | | |
| 26 | tert-Butyl [3-({4-[(6- | 10.31 (s, 1H), 9.43 (s, 1H), | 499 | Method 19 and |
| | pyridin-4-ylquinazolin- | 8.69 (d, 2H), 8.46 (s, 1H), 8.30 (m, | | Method 42 |
| | 2-yl)amino]benzoyl}amino)propyl]carbamate | 2H), 8.08 (d, 2H), 7.85 (m, 5H), | | |
| | | 6.84 (m, 1H), 3.26 (m, 2H), | | |
| | | 2.97 (m, 2H), 1.62 (m, 2H), 1.37 (s, | | |
| | | 9H) | | |
| 27 | N-(2-Piperidin-1- | 10.31 (s, 1H), 9.43 (s, 1H), | 453 | Method 19 and |
| | ylethyl)-4-[(6-pyridin- | 8.68 (d, 2H), 8.46 (s, 1H), 8.28 (m, | | Method 43 |
| | 4-ylquinazolin-2- | 2H), 8.08 (d, 2H), 7.85 (m, 5H), | | |
| | yl)amino]benzamide | 3.37 (m, 2H), 2.39 (m, 6H), | | |
| | | 1.49 (m, 4H), 1.37 (m, 2H) | | |
| 28 | N-[2-(Isopropylamino)ethyl]- | 10.31 (s, 1H), 9.43 (s, 1H), | 427 | Method 7 and |
| | 4-[(6-pyridin-4- | 8.68 (d, 2H), 8.46 (m, 2H), 8.30 (d, | | Method 19 |
| | ylquinazolin-2- | 1H), 8.08 (d, 2H), 7.85 (m, 5H), | | |
| | yl)amino]benzamide | 3.35 (m, 2H), 2.78 (m, 1H), | | |
| | | 2.69 (m, 2H), 0.99 (d, 6H) | | |
| 29 | N-[2-(1- | 10.30 (s, 1H), 9.43 (s, 1H), | 453 | Method 9 and |
| | Methylpyrrolidin-2- | 8.69 (d, 2H), 8.46 (s, 1H), 8.30 (m, | | Method 19 |
| | yl)ethyl]-4-[(6-pyridin- | 2H), 8.07 (d, 2H), 7.85 (m, 5H), | | |
| | 4-ylquinazolin-2- | 3.27 (m, 2H), 2.92 (m, 1H), | | |
| | yl)amino]benzamide | 2.20 (s, 3H), 2.00 (m, 4H), 1.62 (m, | | |
| | | 2H), 1.44 (m, 2H) | | |
| 30 | N,N-Dimethyl-4-[(6- | 10.26 (s, 1H), 9.42 (s, 1H), | 370 | Method 19 and |
| | pyridin-4-ylquinazolin- | 8.68 (d, 2H), 8.45 (s, 1H), 8.29 (m, | | Method 44 |
| | 2-yl)amino]benzamide | 1H), 8.06 (d, 2H), 7.83 (m, 3H), | | |
| | | 7.42 (d, 2H), 2.98 (s, 6H) | | |
| 31 | N-[3-(Dimethylamino)propyl]- | 10.30 (s, 1H), 9.43 (s, 1H), | 427 | Method 10 and |
| | 4-[(6-pyridin- | 8.69 (d, 2H), 8.46 (m, 1H), 8.37 (m, | | Method 19 |
| | 4-ylquinazolin-2- | 1H), 8.30 (m, 1H), 8.07 (d, 2H), | | |
| | yl)amino]benzamide | 7.84 (m, 5H), 3.26 (m, 2H), | | |
| | | 2.29 (m, 2H), 2.16 (s, 6H), 1.66 (m, | | |
| | | 2H) | | |
| 32 | N-(2-Methoxy-1- | 10.30 (s, 1H), 9.43 (s, 1H), | 414 | Method 19 and |
| | methylethyl)-4-[(6- | 8.69 (d, 2H), 8.46 (m, 1H), 8.30 (m, | | Method 45 |
| | pyridin-4-ylquinazolin- | 1H), 8.07 (m, 3H), 7.84 (m, 5H), | | |
| | 2-yl)amino]benzamide | 4.19 (m, 1H), 3.41 (m, 1H), | | |
| | | 3.27 (m, 4H), 1.14 (d, 3H) | | |
| 33 | 2-Methyl-N-(2- | 10.09 (s, 1H), 9.36 (s, 1H), | 469 | Method 19 and |
| | morpholin-4-ylethyl)- | 8.64 (m, 2H), 8.41 (s, 1H), 8.24 (d, | | Method 47 |
| | 4-[(6-pyridin-4- | 1H), 8.02 (m, 1H), 7.89 (d, 1H), | | |
| | ylquinazolin-2- | 7.78 (m, 4H), 7.31 (d, 1H), | | |
| | yl)amino]benzamide | 3.55 (s, 3H), 3.30 (s, 12H) | | |
| 34 | N-(3-Hydroxybutyl)-4- | 10.30 (s, 1H), 9.43 (s, 1H), | 414 | Method 19 and |
| | [(6-pyridin-4- | 8.69 (d, 2H), 8.46 (s, 1H), 8.31 (m, | | Method 48 |
| | ylquinazolin-2- | 2H), 8.06 (d, 2H), 7.84 (m, 5H), | | |
| | yl)amino]benzamide | 4.54 (m, 1H), 3.68 (m, 1H), | | |
| | | 3.31 (m, 1H), 1.57 (m, 2H), 1.08 (d, | | |
| | | 3H) | | |
| 35 | N-[3-(1H-Imidazol-1- | 10.30 (s, 1H), 9.44 (s, 1H), | 450 | Method 19 and |
| | yl)propyl]-4-[(6- | 8.69 (d, 2H), 8.46 (m, 1H), 8.38 (m, | | Method 49 |
| | pyridin-4-ylquinazolin- | 1H), 8.30 (m, 1H), 8.08 (d, 2H), | | |
| | 2-yl)amino]benzamide | 7.84 (m, 5H), 7.67 (s, 1H), | | |
| | | 7.22 (s, 1H), 6.89 (s, 1H), 4.02 (m, | | |
| | | 2H), 3.22 (m, 2H), 1.96 (m, 2H) | | |
| 36 | N-[2-(Dimethylamino)ethyl]- | 10.11 (s, 1H), 9.40 (s, 1H), | 427 | Method 19 and |
| | 2-methyl-4-[(6- | 8.68 (d, 2H), 8.44 (m, 1H), 8.28 (m, | | Method 50 |
| | pyridin-4-ylquinazolin- | 1H), 8.01 (m, 1H), 7.92 (m, 1H), | | |
| | 2-yl)amino]benzamide | 7.82 (m, 4H), 7.34 (d, 1H), | | |
| | | 3.29 (m, 2H), 2.38 (m, 5H), 2.18 (s, | | |
| | | 6H) | | |
| 37 | N-(2-Methoxyethyl)-2- | 10.45 (s, 1H), 9.49 (s, 1H), | 401 | Method 19 and |
| | [(6-pyridin-4-ylquinazolin- | 8.99 (s, 1H), 8.80 (s, 1H), 8.70 (m, | | Method 51 |
| | 2-yl)amino]isonicotinamide | 2H), 8.52 (s, 1H), 8.43 (d, 1H), | | |
| | | 8.37 (m, 1H), 8.34 (m, 1H), | | |
| | | 7.87 (m, 3H), 3.49 (m, 4H), 3.27 (s, | | |
| | | 3H) | | |
| 38 | N-(2-Methoxyethyl)-6- | 10.31 (s, 1H), 9.50 (s, 1H), | 401 | Method 19 and |
| | [(6-pyridin-4- | 8.71 (m, 3H), 8.52 (m, 2H), 8.35 (d, | | Method 52 |
| | ylquinazolin-2- | 1H), 8.02 (t, 1H), 7.89 (m, 3H), | | |
| | yl)amino]pyridine-2- | 7.66 (d, 1H), 3.50 (m, 4H), | | |
| | carboxamide | 3.27 (s, 3H) | | |
| 39¹ | 6-Pyridin-4-yl-N-[4-(2- | 9.86 (s, 1H), 9.33 (s, 1H), | 412 | Method 19 and |
| | pyrrolidin-1-ylethoxy)phenyl]quinazolin- | 8.67 (d, 2H), 8.39 (s, 1H), 8.21 (m, | | 1-[2-(4- |
| | 2- | 1H), 7.83 (m, 4H), 7.70 (d, 1H), | | bromophenoxy)ethyl]pyrrolidine |
| | amine | 6.95 (d, 2H), 4.04 (m, 2H), | | |
| | | 2.79 (m, 2H), 2.53 (m, 4H), 1.68 (m, | | |
| | | 4H) | | |
| 40¹ | N-{4-[2- | 9.87 (s, 1H), 9.33 (s, 1H), | 386 | Method 19 and |
| | (Dimethylamino)ethoxy]phenyl}- | 8.67 (d, 2H), 8.39 (s, 1H), 8.23 (d, | | [2-(4- |
| | 6- | 1H), 7.83 (m, 4H), 7.71 (d, 1H), | | bromophenoxy)ethyl]dimethylamine |
| | pyridin-4-ylquinazolin- | 6.94 (d, 2H), 4.02 (t, 2H), 2.61 (t, | | |
| | 2-amine | 2H), 2.21 (s, 6H) | | |
| 41 | N-(4-Methoxyphenyl)- | 9.86 (s, 1H), 9.33 (s, 1H), | 329 | Method 19 and |
| | 6-pyridin-4- | 8.67 (d, 2H), 8.39 (m, 1H), 8.23 (m, | | 1-bromo-4- |
| | ylquinazolin-2-amine | 1H), 7.86 (d, 2H), 7.82 (d, 2H), | | methoxybenzene |
| | | 7.70 (d, 1H), 6.93 (d, 2H), | | |
| | | 3.74 (s, 3H) | | |
| 42^{1,2} | N-[4-(2-Piperidin-1- | 9.86 (s, 1H), 9.33 (s, 1H), | 426 | Method 19 and |
| | ylethoxy)phenyl]-6- | 8.67 (d, 2H), 8.39 (m, 1H), 8.23 (m, | | 1-[2-(4- |
| | pyridin-4-ylquinazolin- | 3H), 7.83 (m, 4H), 7.71 (d, 1H), | | bromophenoxy)ethyl]piperidine) |
| | 2-amine | 6.93 (d, 2H), 4.04 (m, 2H), | | |
| | | 2.65 (m, 2H), 2.44 (m, 4H), 1.48 (m, | | |
| | | 4H), 1.37 (m, 2H) | | |
| 43¹ | N-[4-(2-Morpholin-4- | 9.84 (s, 1H), 9.33 (s, 1H), | 428 | Method 19 and |
| | ylethoxy)phenyl]-6- | 8.67 (d, 2H), 8.39 (m, 1H), 8.21 (m, | | 4-[2-(4- |
| | pyridin-4-ylquinazolin- | 1H), 7.85 (d, 2H), 7.82 (d, 2H), | | bromophenoxy)ethyl]morpholine |
| | 2-amine | 7.70 (d, 1H), 6.93 (d, 2H), | | |
| | | 4.07 (m, 2H), 3.58 (m, 4H), 2.68 (m, | | |
| | | 2H), 2.46 (m, 4H) | | |
| 44 | N-1,3-Benzodioxol-5- | 9.92 (s, 1H), 9.34 (s, 1H), | 343 | Method 19 and |
| | yl-6-pyridin-4- | 8.67 (d, 2H), 8.39 (d, 1H), 8.23 (m, | | 5-bromo-1,3- |
| | ylquinazolin-2-amine | 1H), 7.82 (d, 2H), 7.73 (m, 2H), | | benzodioxole |
| | | 7.35 (m, 1H), 6.89 (d, 1H), | | |
| | | 5.99 (s, 2H) | | |
| 45 | N-(2,3-Dihydro-1,4- | 9.83 (s, 1H), 9.33 (s, 1H), | 357 | Method 19 and |
| | benzodioxin-6-yl)-6- | 8.67 (m, 2H), 8.39 (d, 1H), 8.23 (m, | | 6-bromo-2,3- |
| | pyridin-4-ylquinazolin- | 1H), 7.82 (m, 2H), 7.71 (d, 1H), | | dihydro-1,4- |
| | 2-amine | 7.67 (m, 1H), 7.34 (m, 1H), | | benzodioxine |
| | | 6.81 (d, 1H), 4.24 (m, 4H) | | |
| 46³ | N-(2-Methoxyethyl)-4- | 10.28 (s, 1H), 9.41 (s, 1H), | 414 | Method 3 and |
| | {[6-(3-methylpyridin- | 8.55 (s, 1H), 8.50 (d, 1H), | | Method 22 |
| | 4-yl)quinazolin-2- | 8.42-8.35 (m, 1H), 8.10-8.02 | | |
| | yl]amino}benzamide | (m, 3H), 7.93-7.78 (m, 4H), | | |
| | | 7.36 (d, 1H), 3.47-3.41 (m, 4H), | | |
| | | 3.27 (s, 3H), 2.32 (s, 3H) | | |
| 47³ | 4-{[6-(3-Chloropyridin- | 10.34 (s, 1H), 9.47 (s, 1H), | 434 | Method 3 and |
| | 4-yl)quinazolin- | 8.80 (s, 1H), 8.65 (d, 1H), | | Method 23 |
| | 2-yl]amino}- | 8.46-8.35 (m, 1H), 8.22-8.15 | | |
| | N-(2- | (m, 1H), 8.09 (d, 2H), | | |
| | methoxyethyl)benzamide | 8.01 (dd, 1H), 7.91-7.81 (m, 3H), | | |
| | | 7.64 (d, 1H), 3.50-3.40 (m, 4H), | | |
| | | 3.28 (s, 3H) | | |
| 48³ | 4-{[6-(3-Fluoropyridin- | 10.36 (s, 1H), 9.48 (s, 1H), | 418 | Method 3 and |
| | 4-yl)quinazolin-2- | 8.73 (s, 1H), 8.57 (d, 1H), 8.40 (s, | | Method 24 |
| | yl]amino}-N-(2- | 1H), 8.34 (s, 1H), | | |
| | methoxyethyl)benzamide | 8.17-8.05 (m, 3H), 7.91-7.83 | | |
| | | (m, 3H), 7.78 (t, 1H), | | |
| | | 3.48-3.39 (m, 4H), 3.28 (s, 3H) | | |
| 49 | N-(4-{1-[(2- | 9.97 (s, 1H), 9.36 (s, 1H), | 422 (M + Na) | Method 99 and |
| | Methoxyethyl)amino]ethyl}phenyl)- | 8.67 (d, 2H), 8.41 (d, 1H), 8.25 (m, | | Method 19 |
| | 6- | 2H), 7.90 (d, 2H), 7.83 (d, 2H), | | |
| | pyridin-4-ylquinazolin- | 7.74 (d, 1H), 7.27 (d, 2H), | | |
| | 2-amine | 3.68 (m, 1H), 3.34 (m, 2H), 3.21 (s, | | |
| | | 3H), 2.43 (m, 2H), 1.25 (d, 3H) | | |
| 50 | N,N,2-Trimethyl-4-(6- | 10.28 (s, 1H), 9.44 (s, 1H), | 384 | Method 19 and |
| | pyridin-4- | 8.99 (d, 1H), 8.73 (s, 1H), 8.48 (m, | | Method 55 |
| | yl)quinazolin-2- | 2H), 7.84 (m, 3H), 7.62 (m, 1H), | | |
| | ylamino)benzamide | 7.26 (m, 2H), 2.99 (s, 3H), | | |
| | | 2.22 (s, 3H), 2.17 (s, 3H) | | |
| 51² | 2-Methyl-N-(2- | 10.52 (m, 1H), 10.35 (m, 1H), | 467 | Method 19 and |
| | (piperidin-1-yl)ethyl)- | 9.45 (s, 1H), 9.0 (d, 2H), 8.78 (s, | | Method 56 |
| | 4-(6-(pyridin-4- | 1H), 8.54 (m, 4H), 7.88 (m, 2H), | | |
| | yl)quinazolin-2- | 7.51 (d, 1H), 3.65 (m, 2H), | | |
| | ylamino)benzamide | 3.5 (m, 2H), 3.19 (m, 2H), 2.92 (m, | | |
| | | 2H), 2.53 (s, 3H), 1.77 (m, 6H), | | |
| | | 1.40 (m, 1H) | | |
| 52 | 2-{4-[(6-Pyridin-4- | 9.94 (s, 1H), 9.35 (s, 1H), | 343 | Method 19 and |
| | ylquinazolin-2- | 8.67 (d, 2H), 8.41 (d, 1H), 8.24 (m, | | 2-(4- |
| | yl)amino]phenyl}ethanol | 1H), 7.85 (m, 4H), 7.73 (d, 1H), | | bromophenyl)ethanol |
| | | 7.18 (d, 2H), 3.58 (m, 2H), | | |
| | | 2.69 (m, 2H) | | |
| 53 | 1-{4-[(6-Pyridin-4- | 10.49 (s, 1H), 9.46 (s, 1H), | 341 | Method 19 and |
| | ylquinazolin-2- | 8.70 (d, 2H), 8.48 (d, 1H), 8.32 (m, | | 1-(4- |
| | yl)amino]phenyl}ethanone | 1H), 8.16 (d, 2H), 7.97 (d, 2H), | | bromophenyl)ethanone |
| | | 7.85 (m, 3H), 2.54 (s, 3H) | | |
| 54 | 1-{4-[(6-Pyridin-4- | 10.02 (s, 1H), 9.37 (s, 1H), | 382 | Method 19 and |
| | ylquinazolin-2- | 8.67 (d, 2H), 8.41 (d, 1H), 8.25 (m, | | 1-(4-bromo- |
| | yl)amino]phenyl}pyrrolidin- | 1H), 7.98 (d, 2H), 7.82 (m, 2H), | | phenyl)pyrrolidin- |
| | 2-one | 7.76 (d, 2H), 7.62 (d, 1H), | | 2-one |
| | | 3.83 (m, 2H), 2.48 (m, 2H), 2.06 (m, | | |
| | | 2H) | | |
| 55² | N-(4-{1-[(3- | 10.05 (s, 1H), 9.38 (s, 1H), | 414 | Method 19 and |
| | Methoxypropyl)amino]ethyl}phenyl)- | 8.68 (d, 2H), 8.42 (d, 1H), 8.26 (m, | | Method 109 |
| | 6- | 2H), 8.21 (s, 1H), 7.96 (d, 2H), | | |
| | pyridin-4-ylquinazolin- | 7.83 (d, 2H), 7.76 (d, 1H), | | |
| | 2-amine | 7.35 (d, 2H), 3.93 (m, 1H), 3.32 (m, | | |
| | | 2H), 3.17 (s, 3H), 2.57 (m, 2H), | | |
| | | 1.67 (m, 2H), 1.37 (d, 3H) | | |
| 56² | 4-[(1-{4-[(6-Pyridin-4- | 10.10 (s, 0.5H), 10.05 (s, 0.5H), | 414 | Method 19 and |
| | ylquinazolin-2- | 9.39 (s, 0.5H), 9.37 (s, 0.5H), | | Method 111 |
| | yl)amino]phenyl}ethyl)amino]butan- | 8.68 (m, 2H), 8.42 (m, 1H), | | |
| | 2-ol | 8.27 (m, 2H), 7.96 (m, 2H), 7.83 (m, | | |
| | | 2H), 7.77 (m, 1H), 7.37 (m, 2H), | | |
| | | 4.27 (m, 1H), 3.83 (m, 1H), | | |
| | | 3.63 (m, 2H), 1.64 (m, 2H), 1.30 (m, | | |
| | | 3H), 0.99 (m, 3H) | | |
| 57 | 2-[(1-{4-[(6-Pyridin-4- | 9.96 (s, 1H), 9.36 (s, 1H), | 386 | Method 19 and |
| | ylquinazolin-2- | 8.67 (d, 2H), 8.41 (d, 1H), 8.24 (m, | | Method 112 |
| | yl)amino]phenyl}ethyl)amino]ethanol | 1H), 7.88 (d, 2H), 7.82 (d, 2H), | | |
| | | 7.75 (d, 1H), 7.28 (d, 2H), | | |
| | | 3.67 (m, 1H), 2.42 (m, 4H), 1.25 (d, | | |
| | | 3H) | | |
| 58 | 3-[(1-{4-[(6-Pyridin-4- | 9.95 (s, 1H), 9.36 (s, 1H), | 400 | Method 19 and |
| | ylquinazolin-2- | 8.67 (d, 2H), 8.41 (d, 1H), 8.25 (m, | | Method 113 |
| | yl)amino]phenyl}ethyl)amino]propan- | 1H), 7.88 (d, 2H), 7.83 (d, 2H), | | |
| | 1-ol | 7.75 (d, 1H), 7.28 (d, 2H), | | |
| | | 3.63 (m, 1H), 3.41 (m, 2H), 2.37 (m, | | |
| | | 2H), 1.52 (m, 2H), 1.24 (d, 3H) | | |

¹The compound was prepared by the procedure of Example 1 but using sodium t-butoxide as the base and a 20-24 hr reaction time.
²Formic acid used in the Gilson HPLC, therefore compounds were isolated as the formic acid salts.
³ Examples were purified by an ISCO system using 100% EtOAc as eluent.

### Example 59

### N-[3-(Methylamino)propyl]-4-[(6-pyridin-4-ylquinazolin-2-yl)amino]benzamide

To a solution of tert-butyl methyl[3-({4-[(6-pyridin-4-ylquinazolin-2 yl)amino]benzoyl}amino)propyl]carbamate (Example 13, 100 mg, 0.195 mmol) dissolved in a minimal amount of MeOH was added 4N HCl in dioxane. The reaction mixture was stirred for 30 minutes. The mixture was then concentrated and dried under reduced pressure to afford the title compound. NMR: 10.49 (s, 1H), 9.48 (s, 1H), 9.00 (d, 2H), 8.76 (br s, 2H), 8.65 (m, 1H), 8.49 (m, 3H), 8.10 (d, 2H), 7.91 (m, 3H), 3.35 (m, 2H), 2.92 (m, 2H), 3.15 (s, 3H), 1.86 (m, 2H); m/z 413. (If final product was not >95% pure, a Gilson HPLC was performed using 0.1% TFA in CH₃CN and water)

### Examples 60-64

The following compounds were prepared by the procedure of Example 59, using the indicated starting materials.

| Ex | Compound | NMR | m/z | SM |
|---|---|---|---|---|
| 60 | 4-[(6-Pyridin-4- | 10.52 (s, 1H), 9.48 (s, 1H), 9.33 (m, 1H), | 425 | Example |
| | ylquinazolin-2- | 8.98 (d, 2H), 8.88 (m, 1H), 8.74 (s, 1H), | | 19 |
| | yl)amino]-N- | 8.46 (d, 3H), 8.13 (d, 2H), 7.94 (m, 2H), | | |
| | pyrrolidin-2- | 3.70 (m, 2H), 3.58 (m, 2H), 3.17 (m, 2H), | | |
| | ylmethylbenzamide | 2.03 (m, 2H), 1.90 (m, 2H) | | |
| 61 | N-(2,3- | 10.32 (s, 1H), 9.43 (s, 1H), 8.69 (m, 2H), | 416 | Example |
| | Dihydroxypropyl)-4- | 8.46 (m, 2H), 8.29 (m, 1H), 8.08 (m, 2H), | | 16 |
| | [(6-pyridin-4- | 7.85 (m, 5H), 3.66 (m, 2H), 3.36 (m, 3H), | | |
| | ylquinazolin-2- | 3.22 (m, 2H) | | |
| | yl)amino]benzamide | | | |
| 62 | N-(3-Aminopropyl)-4- | 10.48 (s, 1H), 9.47 (s, 1H), 8.98 (d, 2H), | 399 | Example |
| | [(6-pyridin-4- | 8.74 (s, 1H), 8.63 (m, 1H), 8.45 (m, 3H), | | 26 |
| | ylquinazolin-2- | 8.10 (m, 2H), 7.91 (m, 5H), 3.34 (m, 2H), | | |
| | yl)amino]benzamide | 2.84 (m, 2H), 1.82 (m, 2H) | | |
| 63 | N-(2-Aminoethyl)-4- | 10.49 (s, 1H), 9.48 (s, 1H), 8.96 (m, 2H), | 385 | Example |
| | [(6-pyridin-4- | 8.73 (s, 1H), 8.67 (m, 1H), 8.44 (m, 3H), | | 25 |
| | ylquinazolin-2- | 8.10 (d, 2H), 8.03 (m, 2H), 7.95 (m, 3H), | | |
| | yl)amino]benzamide | 3.53 (m, 2H), 2.98 (m, 2H) | | |
| 64 | N-(Piperidin-4- | 10.46 (s, 1H), 9.47 (s, 1H), 8.98 (m, 2H), | 439 | Example |
| | ylmethyl)-4-[(6- | 8.73 (m, 2H), 8.45 (m, 4H), 8.09 (d, 2H), | | 24 |
| | pyridin-4-ylquinazolin- | 7.89 (m, 3H), 3.24 (m, 4H), 2.82 (m, 2H), | | |
| | 2-yl)amino]benzamide | 1.81 (m, 3H), 1.38 (m, 2H) | | |

### Example 65

### N-[2-(Methylamino)ethyl]-6-[(6-pyridin-4-ylquinazolin-2-yl)amino]pyridine-2-carboxamide

6-Pyridin-4-ylquinazolin-2-amine (Method 19; 148 mg, 0.667 mmol), tert-butyl (2-{[(6-bromopyridin-2-yl)carbonyl]amino}ethyl)methylcarbamate (Method 53; 239 mg, 0.667 mmol), Cs₂CO₃ (650 mg, 2.00 mmol, 3.0 equiv) and BINAP (82.9 mg, 0.133 mmol) in dioxane (4 ml) were treated with Pd₂(dba)₃ (61.2 mg, 0.0667 mmol). The reaction mixture was heated to 100° C. for 12 h. The reaction was then cooled to room temperature, filtered, and concentrated under reduced pressure. The resulting solid was purified by a Gilson HPLC (0.1% ammonium acetate in CH₃CN and water) followed by subsequent deprotection using 4N HCl in dioxane (5 ml) for one hour. The reaction mixture was concentrated and dried under reduced pressure to afford the title compound. NMR: 10.30 (br s, 1H), 9.57 (s, 1H), 9.01 (m, 4H), 8.82 (s, 1H), 8.70 (m, 1H), 8.54 (m, 1H), 8.48 (m, 2H), 8.11 (m, 1H), 7.99 (d, 1H), 7.78 (m, 1H), 3.65 (m, 2H), 3.13 (m, 2H), 2.58 (s, 3H); m/z 400.

### Example 66-69

The following compounds were prepared by the procedure of Example 65, using the indicated starting materials.

| Ex | Compound | NMR | m/z | SM |
|---|---|---|---|---|
| 66 | N-[2-(Methylamino)ethyl]- | 10.98 (br s, 1H), 9.56 (s, 1H), 9.21 (m, | 400 | Method |
| | 2-[(6-pyridin-4- | 2H), 9.11 (m, 1H), 8.90 (m, 2H), | | 19 and |
| | ylquinazolin-2- | 8.84 (s, 1H), 8.74 (m, 1H), 8.52 (m, 1H), | | Method |
| | yl)amino]isonicotinamide | 8.30 (m, 1H), 7.94 (m, 2H), 7.57 (m, | | 54 |
| | | 1H), 3.60 (m, 2H), 3.13 (m, 2H), | | |
| | | 2.59 (s, 3H) | | |
| 67 | N-Piperidin-4-yl-4-[(6- | 10.30 (s, 1H), 9.43 (s, 1H), 8.68 (d, 2H), | 425 | Method |
| | pyridin-4-ylquinazolin-2- | 8.46 (m, 1H), 8.29 (m, 1H), 8.15 (m, | | 19 and |
| | yl)amino]benzamide | 1H), 8.07 (m, 2H), 7.84 (m, 6H), | | Method |
| | | 3.85 (m, 1H), 3.03 (m, 2H), 2.06 (m, 2H), | | 31 |
| | | 1.74 (m, 2H), 1.51 (m, 2H) | | |
| 68 | N-Piperidin-2-ylmethyl-4- | 10.50 (s, 1H), 9.48 (s, 1H), 8.97 (d, 2H), | 439 | Method |
| | [(6-pyridin-4-ylquinazolin- | 8.82 (m, 1H), 8.73 (s, 2H), 8.43 (m, | | 19 and |
| | 2-yl)amino]benzamide | 3H), 8.12 (d, 2H), 7.95 (m, 2H), | | Method |
| | | 7.92 (m, 1H), 3.47 (m, 2H), 3.23 (m, 2H), | | 37 |
| | | 2.87 (m, 1H), 1.76 (m, 4H), 1.47 (m, | | |
| | | 2H) | | |
| 69 | N-[2-(Methylamino)ethyl]- | 10.50 (s, 1H), 9.48 (s, 1H), 8.96 (d, 2H), | 399 | Method |
| | 4-(6-pyridin-4- | 8.81 (m, 2H), 8.72 (m, 1H), 8.44 (m, | | 19 and |
| | ylquinazolin-2- | 3H), 8.12 (m, 2H), 7.93 (m, 3H), | | Method |
| | yl)benzamide | 3.55 (m, 2H), 3.09 (m, 2H), 2.59 (s, 3H) | | 46 |

### Example 70

### N-(4-{[(2-Morpholin-4-ylethyl)amino]methyl}phenyl)-6-pyridin-4-ylquinazolin-2-amine

4-[(6-Pyridin-4-ylquinazolin-2-yl)amino]benzaldehyde (Example 121, 70 mg, 0.21 mmol) and (2-morpholin-4-ylethyl)amine (30.7 mg, 0.24 mmol) in 5 ml MeOH (with 3 Å molecular sieves) was stirred at room temperature whereupon a few drops of acetic acid was added. NaBH₃CN (22 mg, 1.6 equiv) was then added and the reaction mixture was stirred at room temperature overnight followed by quenching with NaOH (1N, aq., ˜5 ml). The reaction mixture was extracted with EtOAc, and the water layers were then extracted with EtOAc three times. The combined organic layers were washed with water and brine, evaporated and purified by Gilson HPLC (0.1% 10 mM ammonium acetate in CH₃CN and water) to give 52 mg (55%) of the desired product. NMR: 9.99 (s, 1H), 9.36 (s, 1H), 8.67 (d, 2H), 8.41 (d, 1H), 8.25 (m, 1H), 7.91 (d, 2H), 7.83 (d, 2H), 7.76 (d, 1H), 7.27 (d, 2H), 3.66 (m, 2H), 3.54 (m, 4H), 2.58 (m, 2H), 2.38 (m, 2H), 2.31 (m, 4H); m/z 439 (M-H).

### Examples 71-77

The following compounds were prepared by the procedure of Example 70, using the indicated starting materials.

| Ex | Compound | NMR | m/z | SM |
|---|---|---|---|---|
| 71 | N-(4-{[(2- | 9.99 (s, 1H), 9.36 (s, 1H), | 384 (M − H) | Example 121 |
| | Methoxyethyl)amino]methyl}phenyl)- | 8.67 (d, 2H), 8.41 (d, 1H), 8.25 (m, | | and (2- |
| | 6- | 1H), 7.91 (d, 2H), 7.83 (d, 2H), | | methoxyethyl)amine |
| | pyridin-4-ylquinazolin- | 7.75 (d, 1H), 7.27 (d, 2H), | | |
| | 2-amine | 3.66 (s, 2H), 3.39 (m, 2H), 3.23 (s, | | |
| | | 3H), 2.63 (m, 2H) | | |
| 72 | N-[4-({[2-(1- | 9.99 (s, 1H), 9.37 (s, 1H), | 439 | Example 121 |
| | Methylpyrrolidin-2- | 8.67 (d, 2H), 8.41 (d, 1H), 8.25 (m, | | and [2-(1- |
| | yl)ethyl]amino}methyl)phenyl]- | 1H), 7.91 (d, 2H), 7.83 (d, 2H), | | methylpyrrolidin- |
| | 6-pyridin-4- | 7.75 (d, 1H), 7.28 (d, 2H), | | 2- |
| | ylquinazolin-2-amine | 3.67 (s, 2H), 2.90 (m, 1H), 2.18 (s, | | yl)ethyl]amine |
| | | 3H), 2.01 (m, 2H), 1.79 (m, 4H), | | |
| | | 1.58 (m, 2H), 1.35 (m, 2H) | | |
| 73 | N,N-Dimethyl-N′-{4- | 10.00 (s, 1H), 9.38 (s, 1H), | 397 (M − H) | Example 121 |
| | [(6-pyridin-4- | 8.69 (d, 2H), 8.43 (d, 1H), 8.26 (m, | | and N,N- |
| | ylquinazolin-2- | 1H), 7.92 (d, 2H), 7.84 (d, 2H), | | dimethylethane- |
| | yl)amino]benzyl}ethane- | 7.76 (d, 1H), 7.28 (d, 2H), | | 1,2-diamine |
| | 1,2-diamine | 3.67 (s, 2H), 2.54 (m, 2H), 2.33 (m, | | |
| | | 2H), 2.12 (s, 6H) | | |
| 74 | N¹,N¹-Dimethyl-N²-{4- | 9.99 (s, 1H), 9.37 (s, 1H), | 413 | Example 121 |
| | [(6-pyridin-4- | 8.67 (d, 2H), 8.41 (d, 1H), 8.25 (m, | | and N¹,N¹- |
| | ylquinazolin-2- | 1H), 7.91 (d, 2H), 7.83 (d, 2H), | | dimethylpropane- |
| | yl)amino]benzyl}propane- | 7.75 (d, 1H), 7.26 (d, 2H), | | 1,2- |
| | 1,2-diamine | 3.76 (d, 1H), 3.57 (d, 1H), 2.64 (m, | | diamine |
| | | 1H), 2.20 (m, 2H), 2.06 (s, 6H), | | |
| | | 0.93 (d, 3H) | | |
| 75¹ | 2-(Methyl{4-[(6- | 10.02 (s, 1H), 9.37 (s, 1H), | 408 (M + Na) | Example 121 |
| | pyridin-4-ylquinazolin- | 8.67 (d, 2H), 8.41 (d, 1H), 8.25 (m, | | and 2- |
| | 2-yl)amino]benzyl}amino)ethanol | 1H), 8.18 (s, 1H), 7.92 (d, 2H), | | (methylamino) ethanol |
| | | 7.83 (d, 2H), 7.75 (d, 1H), | | |
| | | 7.26 (d, 2H), 3.51 (m, 4H), 2.18 (s, | | |
| | | 3H), 2.45 (m, 2H) | | |
| 76¹ | N-(4-{[(2- | 10.01 (s, 1H), 9.37 (s, 1H), | 422 (M + Na) | Example 121 |
| | Methoxyethyl)(methyl)amino]methyl}phenyl)- | 8.67 (d, 2H), 8.41 (d, 1H), 8.25 (m, | | and (2- |
| | 6-pyridin-4- | 1H), 8.18 (s, 1H), 7.92 (d, 2H), | | methoxyethyl)methylamine |
| | ylquinazolin-2-amine | 7.83 (d, 2H), 7.75 (d, 1H), | | |
| | | 7.24 (d, 2H), 3.66 (s, 2H), 3.47 (m, | | |
| | | 2H), 3.20 (s, 3H), 2.66 (m, 2H), | | |
| | | 2.27 (s, 3H) | | |
| 77¹ | 2-(Ethyl{4-[(6-pyridin- | 10.00 (s, 1H), 9.37 (s, 1H), | 422 (M + Na) | Example 121 |
| | 4-ylquinazolin-2- | 8.67 (d, 2H), 8.41 (d, 1H), 8.25 (m, | | and 2- |
| | yl)amino]benzyl}amino)ethanol | 1H), 8.15 (s, 1H), 7.92 (d, 2H), | | (ethylamino)ethanol |
| | | 7.83 (d, 2H), 7.75 (d, 1H), | | |
| | | 7.27 (d, 2H), 3.57 (s, 2H), 3.46 (m, | | |
| | | 2H), 3.32 (m, 4H), 1.00 (t, 3H) | | |

¹Compound was prepared by the procedure of Example 70 but using a reaction temperature of 30° C. for 24 hours or until TLC indicated all starting material was consumed.

### Example 78

### {4-[(6-Pyridin-4-ylquinazolin-2-yl)amino]phenyl}methanol

4-[(6-Pyridin-4-ylquinazolin-2-yl)amino]benzaldehyde (Example 121, 53 mg, 0.162 mmol) was dissolved in 5 ml MeOH (with 3 Å molecular sieves) and stirred at room temperature whereupon NaBH₄ (9.8 mg, 0.259 mmol) was then added and the reaction mixture was stirred at room temperature overnight followed by quenching with NaOH (1N, aq., ˜5 ml). The reaction mixture was extracted with EtOAc, organic layers washed with water and brine, evaporated and purified by Gilson HPLC (0.1% ammonium acetate in CH₃CN and water) to afford the desired product. NMR: 9.98 (s, 1H), 9.37 (s, 1H), 8.67 (d, 2H), 8.41 (d, 1H), 8.24 (m, 1H), 7.93 (d, 2H), 7.82 (d, 2H), 7.76 (d, 1H), 7.28 (d, 2H), 4.45 (s, 2H); m/z 329.

### Example 79

### N-(4-Morpholin-4-ylphenyl)-6-pyridin-4-ylquinazolin-2-amine

6-Bromo-N-(4-morpholin-4-ylphenyl)quinazolin-2-amine (Method 62, 117 mg, 0.304 mmol, 1.0 equiv), pyridine-4-ylboronic acid (55.9 mg, 0.456 mmol, 1.5 equiv) and caesium carbonate (296 mg, 0.912 mmol, 3.0 equiv) in dioxane/water (4:1, 4 ml) were treated with Pd(PPh₃)₄ (35.1 mg, 0.0304 mmol, 0.1 equiv). The reaction was stirred at 80° C. for 12 h. The reaction was then concentrated under reduced pressure and purified by Gilson HPLC (0.1% ammonium acetate in CH₃CN and water) to afford 40 mgs (35% yield) of the desired product. NMR: 9.81 (s, 1H), 9.31 (s, 1H), 8.66 (d, 2H), 8.38 (s, 1H), 8.22 (d, 1H), 7.81 (m, 4H), 7.69 (d, 1H), 6.95 (d, 2H), 3.73 (m, 4H), 3.06 (m, 4H); m/z 384.

### Examples 80-109

The following compounds were prepared by the procedure of Example 79, using the indicated starting materials.

| Ex | Compound | NMR | m/z | SM |
|---|---|---|---|---|
| 80 | N-{4-[(6-Pyridin-4- | 9.96 (s, 1H), 9.87 (s, 1H), | 356 | Method 63 and |
| | ylquinazolin-2- | 9.35 (s, 1H), 8.68 (m, 2H), | | pyridine-4- |
| | yl)amino]phenyl}acetamide | 8.40 (s, 1H), 8.25 (m, 1H), | | ylboronic acid |
| | | 7.86 (m, 5H), 7.53 (m, 2H), | | |
| | | 2.02 (s, 3H) | | |
| 81 | 4-[(6-Pyridin-4- | 9.73 (s, 1H), 9.30 (s, 1H), | 315 | Method 64 and |
| | ylquinazolin-2- | 9.12 (s, 1H), 8.66 (d, 2H), | | pyridine-4- |
| | yl)amino]phenol | 8.37 (s, 1H), 8.21 (d, 1H), | | ylboronic acid |
| | | 7.82 (d, 2H), 7.71 (m, 3H), | | |
| | | 6.75 (d, 2H) | | |
| 82 | 6-Pyridin-4-yl-N-[3- | 10.40 (s, 1H), 9.45 (s, 1H), | 367 | Method 65 and |
| | (trifluoromethyl)phenyl]quinazolin- | 8.69 (d, 2H), 8.49 (d, 2H), | | pyridine-4- |
| | 2-amine | 8.29 (m, 2H), 7.84 (m, 3H), | | ylboronic acid |
| | | 7.58 (t, 1H), 7.34 (d, 1H) | | |
| 83 | N-(3-Methylphenyl)-6- | 9.96 (s, 1H), 9.37 (s, 1H), | 313 | Method 66 and |
| | pyridin-4-ylquinazolin- | 8.68 (d, 2H), 8.42 (s, 1H), | | pyridine-4- |
| | 2-amine | 8.26 (d, 1H), 7.84 (m, 3H), | | ylboronic acid |
| | | 7.77 (m, 2H), 7.22 (t, 1H), | | |
| | | 6.83 (d, 1H), 2.32 (s, 3H) | | |
| 84 | N-Butyl-3-[(6-pyridin- | 10.39 (s, 1H), 9.44 (s, 1H), | 434 | Method 67 and |
| | 4-ylquinazolin-2- | 8.69 (d, 3H), 8.47 (s, 1H), | | pyridine-4- |
| | yl)amino]benzenesulfonamide | 8.32 (d, 1H), 8.12 (d, 1H), | | ylboronic acid |
| | | 7.85 (m, 3H), 7.57 (m, 2H), | | |
| | | 7.41 (m, 1H), 2.82 (m, 2H), | | |
| | | 1.38 (m, 2H), 1.26 (m, 2H), | | |
| | | 0.79 (t, 3H) | | |
| 85 | N-(3-Morpholin-4- | 9.89 (s, 1H), 9.37 (s, 1H), | 384 | Method 68 and |
| | ylphenyl)-6-pyridin-4- | 8.68 (d, 2H), 8.42 (s, 1H), | | pyridine-4- |
| | ylquinazolin-2-amine | 8.25 (d, 1H), 7.83 (d, 2H), | | ylboronic acid |
| | | 7.75 (m, 2H), 7.43 (d, 1H), | | |
| | | 7.18 (t, 1H), 6.62 (d, 1H), | | |
| | | 3.78 (m, 4H), 3.13 (m, 4H) | | |
| 86 | N-(3-Isopropoxy | 9.99 (s, 1H), 9.38 (s, 1H), | 357 | Method 69 and |
| | phenyl)-6-pyridin-4- | 8.68 (d, 2H), 8.42 (s, 1H), | | pyridine-4- |
| | ylquinazolin-2-amine | 8.26 (d, 1H), 7.82 (d, 2H), | | ylboronic acid |
| | | 7.75 (m, 2H), 7.48 (d, 1H), | | |
| | | 7.20 (t, 1H), 6.55 (d, 1H), | | |
| | | 4.60 (m, 1H), 1.32 (d, 6H) | | |
| 87 | N-(3-Methoxy-4- | 9.93 (s, 1H), 9.36 (s, 1H), | 343 | Method 70 and |
| | methylphenyl)-6- | 8.68 (d, 2H), 8.42 (s, 1H), | | pyridine-4- |
| | pyridin-4-ylquinazolin- | 8.25 (d, 1H), 7.84 (m, 3H), | | ylboronic acid |
| | 2-amine | 7.76 (d, 1H), 7.41 (d, 1H), | | |
| | | 7.07 (d, 1H), 3.83 (s, 3H), | | |
| | | 2.11 (s, 3H) | | |
| 88 | N-(1-Methyl-1H- | 10.16 (s, 1H), 9.32 (s, 1H), | 303 | Method 71 and |
| | pyrazol-3-yl)-6-pyridin- | 8.66 (d, 2H), 8.40 (s, 1H), | | pyridine-4- |
| | 4-ylquinazolin-2-amine | 8.23 (m, 1H), 7.82 (d, 2H), | | ylboronic acid |
| | | 7.72 (m, 1H), 7.61 (s, 1H), | | |
| | | 6.90 (s, 1H), 3.77 (s, 3H) | | |
| 89 | N-(4-Piperidin-1- | 9.77 (s, 1H), 9.30 (s, 1H), | 382 | Method 72 and |
| | ylphenyl)-6-pyridin-4- | 8.66 (d, 2H), 8.37 (s, 1H), | | pyridine-4- |
| | ylquinazolin-2-amine | 8.21 (m, 1H), 7.80 (m, 4H), | | ylboronic acid |
| | | 7.67 (d, 1H), 6.92 (d, 2H), | | |
| | | 3.06 (m, 4H), 1.63 (m, 4H), | | |
| | | 1.51 (m, 2H) | | |
| 90 | N-(6-Morpholin-4- | 9.83 (s, 1H), 9.33 (s, 1H), | 385 | Method 73 and |
| | ylpyridin-3-yl)-6- | 8.71 (m, 1H), 8.67 (d, 2H), | | pyridine-4- |
| | pyridin-4-ylquinazolin- | 8.40 (s, 1H), 8.22 (m, 1H), | | ylboronic acid |
| | 2-amine | 8.09 (m, 1H), 7.82 (m, 2H), | | |
| | | 7.70 (d, 1H), 6.89 (d, 1H), | | |
| | | 3.72 (m, 4H), 3.38 (m, 4H) | | |
| 91 | N-Methyl-N-{4-[(6- | 10.20 (s, 1H), 9.41 (s, 1H), | 370 | Method 74 and |
| | pyridin-4-ylquinazolin- | 8.68 (d, 2H), 8.45 (s, 1H), | | pyridine-4- |
| | 2-yl)amino]phenyl}acetamide | 8.28 (m, 1H), 8.06 (d, 2H), | | ylboronic acid |
| | | 7.82 (m, 3H), 7.29 (d, 2H), | | |
| | | 3.14 (s, 3H), 1.78 (s, 3H) | | |
| 92 | 2-(Ethyl{4-[(6-pyridin- | 9.63 (s, 1H), 9.26 (s, 1H), | 386 | Method 75 and |
| | 4-ylquinazolin-2- | 8.66 (d, 2H), 8.35 (s, 1H), | | pyridine-4- |
| | yl)amino]phenyl}amino)ethanol | 8.20 (d, 1H), 7.81 (d, 2H), | | ylboronic acid |
| | | 7.67 (m, 3H), 6.68 (d, 2H), | | |
| | | 4.67 (m, 1H), 3.53 (m, 2H), | | |
| | | 3.34 (m, 4H), 1.07 (m, 3H) | | |
| 93 | 6-Pyridin-4-yl-N-[3- | 10.41 (s, 1H), 9.45 (s, 1H), | 432 | Method 76 and |
| | (pyrrolidin-1- | 8.68 (m, 3H), 8.47 (m, 1H), | | pyridine-4- |
| | ylsulfonyl)phenyl]quinazolin- | 8.32 (m, 1H), 8.18 (d, 1H), | | ylboronic acid |
| | 2-amine | 7.84 (d, 2H), 7.73 (d, 1H), | | |
| | | 7.59 (t, 1H), 7.49 (d, 1H), | | |
| | | 3.23 (m, 4H), 1.68 (m, 4H) | | |
| 94 | N-[4-(Morpholin-4- | 10.62 (s, 1H), 9.49 (s, 1H), | 448 | Method 77 and |
| | ylsulfonyl)phenyl]-6- | 8.70 (d, 2H), 8.50 (s, 1H), | | pyridine-4- |
| | pyridin-4-ylquinazolin- | 8.31 (m, 3H), 7.86 (m, 3H), | | ylboronic acid |
| | 2-amine | 7.70 (d, 2H), 3.63 (m, 4H), | | |
| | | 2.85 (m, 4H) | | |
| 95 | N-{4-[(Difluoromethyl)sulfonyl]phenyl}- | 10.83 (s, 1H), 9.52 (s, 1H), | 413 | Method 78 and |
| | 6- | 8.70 (d, 2H), 8.52 (d, 1H), | | pyridine-4- |
| | pyridin-4-ylquinazolin- | 8.36 (m, 3H), 7.88 (m, 5H), | | ylboronic acid |
| | 2-amine | 7.22 (t, 1H) | | |
| 96 | 6-Pyridin-4-yl-N-[4- | 10.56 (s, 1H), 9.47 (s, 1H), | 432 | Method 79 and |
| | (pyrrolidin-1- | 8.69 (d, 2H), 8.49 (d, 1H), | | pyridine-4- |
| | ylsulfonyl)phenyl]quinazolin- | 8.33 (m, 1H), 8.26 (m, 2H), | | ylboronic acid |
| | 2-amine | 7.86 (m, 3H), 7.77 (d, 2H), | | |
| | | 3.13 (m, 4H), 1.64 (m, 4H) | | |
| 97 | N-(4-Ethoxyphenyl)-6- | 9.85 (s, 1H), 9.33 (s, 1H), | 343 | Method 80 and |
| | pyridin-4-ylquinazolin- | 8.67 (d, 2H), 8.39 (d, 1H), | | pyridine-4- |
| | 2-amine | 8.22 (m, 1H), 7.83 (m, 4H), | | ylboronic acid |
| | | 7.70 (d, 1H), 6.91 (d, 2H), | | |
| | | 4.00 (m, 2H), 1.32 (m, 3H) | | |
| 98 | N-(3-Fluorophenyl)-6- | 10.28 (s, 1H), 9.43 (s, 1H), | 317 | Method 81 and |
| | pyridin-4-ylquinazolin- | 8.68 (d, 2H), 8.45 (d, 1H), | | pyridine-4- |
| | 2-amine | 8.29 (m, 1H), 8.09 (d, 1H), | | ylboronic acid |
| | | 7.83 (m, 3H), 7.69 (d, 1H), | | |
| | | 7.36 (m, 1H), 6.81 (m, 1H) | | |
| 99 | 6-Pyridin-4-yl-N-[4- | 10.25 (s, 1H), 9.42 (s, 1H), | 383 | Method 82 and |
| | (trifluoromethoxy)phenyl]quinazolin- | 8.68 (d, 2H), 8.45 (d, 1H), | | pyridine-4- |
| | 2- | 8.29 (m, 1H), 8.09 (d, 2H), | | ylboronic acid |
| | amine | 7.83 (m, 3H), 7.36 (d, 2H) | | |
| 100 | N-[3-(Piperidin-1- | 10.42 (s, 1H), 9.45 (s, 1H), | 446 | Method 83 and |
| | ylsulfonyl)phenyl]-6- | 8.69 (d, 2H), 8.61 (s, 1H), | | pyridine-4- |
| | pyridin-4-ylquinazolin- | 8.47 (m, 1H), 8.32 (m, 1H), | | ylboronic acid |
| | 2-amine | 8.16 (d, 1H), 7.84 (d, 2H), | | |
| | | 7.73 (d, 1H), 7.59 (m, 1H), | | |
| | | 7.33 (d, 1H), 2.97 (m, 4H), | | |
| | | 1.57 (m, 4H), 1.37 (m, 2H) | | |
| 101 | N-[3-Methoxy-5- | 10.36 (s, 1H), 9.45 (s, 1H), | 397 | Method 84 and |
| | (trifluoromethyl)phenyl]- | 8.69 (d, 2H), 8.47 (m, 1H), | | pyridine-4- |
| | 6-pyridin-4- | 8.30 (m, 1H), 8.07 (s, 1H), | | ylboronic acid |
| | ylquinazolin-2-amine | 7.97 (s, 1H), 7.82 (m, 3H), | | |
| | | 6.86 (s, 1H), 3.86 (s, 3H) | | |
| 102 | N-[3-(Morpholin-4- | 10.45 (s, 1H), 9.45 (s, 1H), | 448 | Method 85 and |
| | ylsulfonyl)phenyl]-6- | 8.69 (d, 2H), 8.62 (s, 1H), | | pyridine-4- |
| | pyridin-4-ylquinazolin- | 8.47 (m, 1H), 8.31 (m, 1H), | | ylboronic acid |
| | 2-amine | 8.19 (d, 1H), 7.84 (d, 2H), | | |
| | | 7.75 (d, 1H), 7.62 (t, 1H), | | |
| | | 7.35 (d, 1H), 3.66 (m, 4H), | | |
| | | 2.96 (m, 4H) | | |
| 103 | N-[4-(Methylsulfonyl)phenyl]- | 10.58 (s, 1H), 9.48 (s, 1H), | 377 | Method 86 and |
| | 6-pyridin-4- | 8.69 (d, 2H), 8.50 (s, 1H), | | pyridine-4- |
| | ylquinazolin-2-amine | 8.33 (m, 1H), 8.26 (d, 2H), | | ylboronic acid |
| | | 7.86 (m, 4H), 7.57 (m, 1H), | | |
| | | 3.17 (s, 3H) | | |
| 104 | N-[3-(Methylsulfonyl)phenyl]- | 10.46 (s, 1H), 9.46 (s, 1H), | 377 | Method 87 and |
| | 6-pyridin-4- | 8.69 (m, 3H), 8.48 (s, 1H), | | pyridine-4- |
| | ylquinazolin-2-amine | 8.31 (m, 2H), 7.84 (m, 2H), | | ylboronic acid |
| | | 7.63 (m, 3H), 3.23 (s, 3H) | | |
| 105 | N-(4-Fluorophenyl)-6- | 10.10 (s, 1H), 9.89 (s, 1H), | 317 | Method 88 and |
| | pyridin-4-ylquinazolin- | 8.69 (d, 2H), 8.44 (s, 1H), | | pyridine-4- |
| | 2-amine | 8.27 (d, 1H), 8.00-8.04 (m, | | ylboronic acid |
| | | 2H), 7.85 (d, 2H), 7.77 (d, | | |
| | | 1H), 7.20 (t, 2H) | | |
| 106 | N-(4-Methylphenyl)-6- | 9.95 (s, 1H), 9.37 (s, 1H), | 313 | Method 89 and |
| | pyridin-4-ylquinazolin- | 8.69 (d, 2H), 8.42 (s, 1H), | | pyridine-4- |
| | 2-amine | 8.25 (dd, 1H), 7.83-7.89 (m, | | ylboronic acid |
| | | 4H), 7.75 (d, 1H), 7.16 (d, | | |
| | | 2H), 2.29 (s, 3H) | | |
| 107 | N,N-Dimethyl-N′-(6- | 9.71 (s, 1H), 9.30 (s, 1H), | 342 | Method 90 and |
| | pyridin-4-ylquinazolin- | 8.67 (d, 2H), 8.88 (s, 1H), | | pyridine-4- |
| | 2-yl)benzene-1,4- | 8.21 (d, 1H), 7.81 (d, 1H), | | ylboronic acid |
| | diamine | 7.78 (d, 1H), 7.59-7.69 (m, | | |
| | | 3H), 6.77 (d, 2H), 2.88 (s, | | |
| | | 6H) | | |
| 108¹ | N-[4-(1H-Pyrazol-1- | 10.22 (s, 1H), 9.42 (s, 1H), | 365 | Method 91 and |
| | yl)phenyl]-6-pyridin-4- | 8.69 (d, 2H), 8.44 (d, 2H), | | pyridine-4- |
| | ylquinazolin-2-amine | 8.28 (d, 1H), 8.13 (d, 2H), | | ylboronic acid |
| | | 7.80-7.86 (m, 5H), 7.72 (s, | | |
| | | 1H), 6.54 (m, 1H) | | |
| 109 | N-(2-Methyl-1,3- | 10.29 (s, 1H), 9.41 (s, 1H), | 354 | Method 92 and |
| | benzoxazol-5-yl)-6- | 8.68 (bs, 3H), 8.45 (s, 1H), | | pyridine-4- |
| | pyridin-4-ylquinazolin- | 8.29 (dd, 1H), 7.83-7.86 (m, | | ylboronic acid |
| | 2-amine | 3H), 7.63 (dd, 2H), 2.59 (s, | | |
| | | 3H) | | |

¹NMR taken in THF-d₈.

### Example 110

### 3-Methoxy-N-(4-(6-(pyridin-4-yl)quinazolin-2-ylamino)phenyl)propanamide

To a 100 mL round bottom flask was added N-(4-(6-bromoquinazolin-2-ylamino)phenyl)-3-methoxypropanamide (Method 98; 53.0 mg, 0.13 mmol), potassium carbonate (45.6 mg, 0.33 mmol), pyridin-4-ylboronic acid (19.5 mg, 0.16 mmol) and PdCl₂(dppf).CH₂Cl₂ (5.09 mg, 6.23 μmol) in DME (3.00 ml) and water (1.00 ml). The reaction mixture was degassed with argon and heated at 100° C. overnight. The reaction mixture was then filtered and the filtrate was evaporated under reduced pressure. The crude material was purified using an ISCO system (0-10% MeOH in DCM) to yield a yellow solid (16.2 mg, 0.04 mmol, 30.6% yield). NMR: 10.17 (s, 1H), 9.98 (s, 1H), 9.39 (s, 1H), 8.99 (d, 2H), 8.72 (s, 1H), 8.50 (d, 2H), 8.43 (d, 1H), 7.90 (d, 2H), 7.81 (d, 1H), 7.62-7.55 (m, 2H), 3.61 (t, 2H), 3.24 (s, 3H), 2.54 (t, 2H); m/z 400.

### Examples 111-115

The following compounds were prepared by the procedure of Example 110, using the indicated starting materials.

| Ex | Compound | NMR | m/z | SM |
|---|---|---|---|---|
| 111 | N-(2-Methoxyethyl)- | 9.86 (s, 1H), 9.30 (s, 1H), | 386 | Method 97 and |
| | N-methyl-N′-(6- | 8.78 (d, 2H), 8.49 (s, 1H), 8.28 (d, | | pyridin-4- |
| | pyridin-4-yl | 1H), 8.08 (d, 2H), 7.80 (s, 2H), | | ylboronic acid |
| | quinazolin-2-yl)benzene- | 7.69 (d, 1H), 6.83 (s, 2H), | | |
| | 1,4-diamine | 3.48 (bs, 7H), 2.94 (s, 3H) | | |
| 112 | N-[4-(2- | 9.88 (s, 1H), 9.32 (s, 1H), | 373 | Method 93 and |
| | Methoxyethoxy)phenyl]- | 8.66 (s, 2H), 8.39 (s, 1H), 8.22 (d, | | pyridin-4- |
| | 6-pyridin-4- | 1H), 7.90-7.80 (m, 3H), | | ylboronic acid |
| | ylquinazolin-2-amine | 7.75-7.64 (m, 2H), 6.94 (d, 2H), | | |
| | | 4.10-4.03 (m, 2H), 3.65 (t, 2H), | | |
| | | 3.31 (s, 3H) | | |
| 113 | N-(2-Methoxyethyl)- | 10.49 (s, 1H), 9.44 (s, 1H), | 436 | Method 94 and |
| | 4-[(6-pyridin-4- | 8.68 (s, 2H), 8.46 (s, 1H), 8.29 (d, | | pyridin-4- |
| | ylquinazolin-2- | 1H), 8.19 (d, 2H), | | ylboronic acid |
| | yl)amino]benzenesulfonamide | 7.87-7.72 (m, 5H), 7.57 (t, 1H), 3.30 (t, | | |
| | | 2H), 3.16 (s, 3H), 2.94-2.84 (m, | | |
| | | 2H) | | |
| 114 | 3-Methoxy-N-(2- | 9.43 (s, 1H), 8.71-8.65 (m, | 430 | Method 95 and |
| | methoxyethyl)-4-(6- | 3H), 8.52-8.45 (m, 3H), | | pyridin-4- |
| | (pyridin-4- | 8.30 (d, 1H), 7.87-7.82 (m, 3H), | | ylboronic acid |
| | yl)quinazolin-2- | 7.57 (d, 2H), 3.96 (s, 3H), | | |
| | ylamino)benzamide | 3.49-3.42 (m, 4H), 3.28 (s, 3H) | | |
| 115 | N-(2-Methoxyethyl)- | 10.30 (s, 1H), 9.42 (s, 1H), | 430 | Method 96 and (2- |
| | 4-(6-(2- | 8.44 (s, 1H), 8.38 (t, 1H), | | methoxypyridin- |
| | methoxypyridin-4- | 8.30-8.25 (m, 2H), 8.07 (d, 2H), 7.85 (d, | | 4-yl)boronic acid |
| | yl)quinazolin-2- | 2H), 7.81 (d, 1H), 7.45 (d, 1H), | | |
| | ylamino)benzamide | 7.26 (s, 1H), 3.91 (s, 3H), | | |
| | | 3.26 (s, 3H) | | |

### Example 116

### N-{4-[(4-Methyl-1,4-diazepan-1-yl)carbonyl]phenyl}-6-pyridin-4-ylquinazolin-2-amine

To a 25 mL round-bottom flask was added 4-(6-(pyridin-4-yl)quinazolin-2-ylamino)benzoic acid (Example 122, 125 mg, 0.37 mmol) and DMF (4 ml). Pyridine (0.148 mL, 1.83 mmol) and HATU (167 mg, 0.44 mmol) were then added and the reaction was stirred at 50° C. for thirty minutes. 1-Methyl-1,4-diazepane (62.5 mg, 0.55 mmol) was added and the reaction was stirred at 20° C. overnight. The crude reaction mixture was partitioned between EtOAc and water. The organic phase was retained and washed with water, dried over sodium sulfate, filtered, and concentrated under reduced pressure. The crude residue was purified by Gilson HPLC (0.1% TFA in acetonitrile and water) to afford 160 mgs (11.4% yield) of the title compound. NMR (THF-d8): 9.36 (s, 1H), 9.24 (s, 1H), 8.64 (dd, 2H), 8.24 (d, 1H), 8.19 (dd, 1H), 8.05 (d, 2H), 7.84 (d, 1H), 7.71 (dd, 2H), 7.41 (d, 2H), 3.64 (bs, 4H), 2.55 (bs, 4H), 2.32 (s, 3H), 1.87 (bs, 2H); m/z 439.

### Examples 117-120

The following compounds were prepared by the procedure of Example 116, using the indicated starting materials. The reported NMR assignments are all in THF-d₈.

| Ex | Compound | NMR | m/z | SM |
|---|---|---|---|---|
| 117 | N-(4-{[3- | 9.27 (s, 1H), 9.14 (s, 1H), | 439 | Example 122 and |
| | (Dimethylamino)pyrrolidin- | 8.53 (d, 2H), 8.13 (s, 1H), | | N,N- |
| | 1-yl]carbonyl}phenyl)- | 8.09 (d, 1H), 7.95 (d, 2H), | | dimethylpyrrolidin- |
| | 6-pyridin-4- | 7.76 (d, 1H), 7.60 (d, 2H), | | 3-amine |
| | ylquinazolin-2-amine | 7.46 (d, 2H), 3.46 (s, 6H), | | |
| | | 2.50 (bs, 1H), 2.06 (bs, 6H) | | |
| 118 | N-(4-{[(3S)-3- | 9.29 (s, 1H), 9.13 (s, 1H), | 439 | Example 122 and |
| | (Dimethylamino)pyrrolidin- | 8.53 (d, 2H), 8.13 (s, 1H), | | (3S)—N,N- |
| | 1-yl]carbonyl}phenyl)- | 8.08 (d, 1H), 7.96 (d, 2H), | | dimethylpyrrolidin- |
| | 6-pyridin-4- | 7.72 (d, 1H), 7.60 (d, 2H), | | 3-amine |
| | ylquinazolin-2-amine | 7.47 (d, 2H), 3.47 (s, 6H), | | |
| | | 2.52 (bs, 1H), 2.09 (bs, 6H) | | |
| 119 | N-{4-[(4- | 9.83 (s, 1H), 9.13 (s, 1H), | 453 | Example 122 and |
| | Acetylpiperazin-1- | 8.52 (d, 2H), 8.12 (s, 1H), | | 1-acetylpiperazine |
| | yl)carbonyl]phenyl}-6- | 8.07 (dd, 1H), 7.97 (d, 2H), | | |
| | pyridin-4-ylquinazolin- | 7.70 (d, 1H), 7.60 (d, 2H), | | |
| | 2-amine | 7.35 (d, 2H), 3.38-3.52 (m, | | |
| | | 8H), 1.91 (s, 3H) | | |
| 120 | N-{4-[(4- | 9.44 (s, 1H), 9.28 (s, 1H), | 425 | Example 122 and |
| | Methylpiperazin-1- | 8.68 (d, 2H), 8.28 (s, 1H), | | 1- |
| | yl)carbonyl]phenyl}-6- | 8.23 (d, 1H), 8.10 (d, 2H), | | methylpiperazine |
| | pyridin-4-ylquinazolin- | 7.86 (d, 1H), 7.75 (d, 2H), | | |
| | 2-amine | 7.47 (d, 2H), 3.69 (bs, 4H), | | |
| | | 2.52 (bs, 4H), 2.37 (s, 3H) | | |

### Example 121

### 4-[(6-Pyridin-4-ylquinazolin-2-yl)amino]benzaldehyde

6-Pyridin-4-ylquinazolin-2-amine (Method 19, 2 g, 9.0 mmol), 4-bromobenzaldehyde (1.83 g, 9.9 mmol), Cs₂CO₃ (8.8 g, 27 mmol, 3.0 equiv), and BINAP (1.12 g, 1.8 mmol, 0.2 equiv) in dioxane (60 ml) were treated with Pd₂(dba)₃ (825 mg, 0.9 mmol). The reaction mixture was heated to 100° C. for 3 h. The reaction was cooled and filtered. The crude mixture was purified on an ISCO system (EtOAc/Et₃N) to give 1.5 g (51%) of the desired product. NMR: 10.61 (s, 1H), 9.87 (s, 1H), 9.48 (s, 1H), 8.69 (m, 2H), 8.49 (m, 1H), 8.33 (m, 1H), 8.25 (d, 2H), 7.87 (m, 5H); m/z 327.

### Example 122

### 4-(6-(Pyridin-4-yl)quinazolin-2-ylamino)benzoic acid

Methyl 4-[(6-pyridin-4-ylquinazolin-2-yl)amino]benzoate (Example 9, 1.40 g, 3.93 mmol) was dissolved in MeOH-THF-water (1:1:1) and treated with potassium hydroxide (1.32 g, 23.6 mmol). The reaction was stirred at 60° C. for 1 hour whereupon TLC indicated that the reaction was complete. The pH of the reaction mixture was adjusted to 7 with the addition of aqueous HCl. Upon filtration, a yellow solid was obtained and dried in a vacuum oven at 80° C. to afford 1.35 grams (82% yield) of the title compound. NMR: 12.53 (bs, 1H), 10.46 (s, 1H), 9.47 (s, 1H), 8.72 (bs, 2H), 8.50 (s, 1H), 8.33 (d, 1H), 8.14 (d, 2H), 7.86-7.95 (m, 5H); m/z 341 (M-H).

### Example 123

### 3-Methoxy-N-(1-{4-[(6-pyridin-4-ylquinazolin-2-yl)amino]phenyl}ethyl)propanamide

6-Pyridin-4-ylquinazolin-2-amine (Method 19; 214 mg, 0.960 mmol), N-(1-(4-bromophenyl)ethyl)-3-methoxypropanamide (Method 17; 275 mg, 0.960 mmol), Cs₂CO₃ (939 mg, 2.88 mmol, 3.0 equiv) and XANTPHOS (111 mg, 0.190 mmol) in dioxane (4 ml) were treated with palladium (II) acetate (22 mg, 0.10 mmol). The reaction mixture was heated in a microwave at 160° C. for 1 h. The reaction was then purified by a Gilson HPLC (0.1% ammonium acetate in CH₃CN and water) to afford 110 mgs of the desired product (27% yield). NMR: 9.98 (s, 1H), 9.36 (s, 1H), 8.68 (d, 2H), 8.42 (s, 1H), 8.25 (d, 2H), 7.89 (d, 2H), 7.84 (d, 2H), 7.76 (d, 1H), 7.27 (d, 2H), 4.9 (m, 1H), 3.52 (t, 2H), 3.21 (s, 3H), 2.35 (t, 2H), 1.34 (s, 3H); m/z 428.

### Examples 124-138

The following compounds were prepared by the procedure of Example 123, using the indicated starting materials.

| Ex | Compound | NMR | m/z | SM |
|---|---|---|---|---|
| 124 | 3-Methoxy-N-((1R)-1- | 9.98 (s, 1H), 9.36 (s, 1H), 8.67 (d, | 428 | Method |
| | {4-[(6-pyridin-4- | 2H), 8.41 (s, 1H), 8.24 (d, 2H), | | 19 and |
| | ylquinazolin-2- | 7.89 (d, 2H), 7.83 (d, 2H), 7.75 (d, 1H), | | Method |
| | yl)amino]phenyl}ethyl)propanamide | 7.27 (d, 2H), 4.90 (m, 1H), 3.52 (t, | | 61 |
| | | 2H), 3.21 (s, 3H), 2.35 (t, 2H), | | |
| | | 1.34 (d, 3H) | | |
| 125 | 3-Methoxy-N-methyl-N- | 10.2 (s, 1H), 9.41 (s, 1H), 8.68 (d, | 414 | Method |
| | (4-(6-(pyridin-4- | 2H), 8.44 (s, 1H), 8.28 (d, 1H), | | 19 and |
| | yl)quinazolin-2- | 8.06 (d, 2H), 7.83 (m, 3H), 7.28 (d, 2H), | | Method |
| | ylamino)phenyl)propanamide | 3.48 (t, 2H), 3.14 (m, 6H), 2.27 (t, | | 59 |
| | | 2H) | | |
| 126 | 3-Methoxy-N-((1S)-1- | 9.98 (s, 1H), 9.36 (s, 1H), 8.67 (d, | 428 | Method |
| | {4-[(6-pyridin-4- | 2H), 8.41 (s, 1H), 8.25 (d, 2H), | | 19 and |
| | ylquinazolin-2- | 7.89 (d, 2H), 7.83 (d, 2H), 7.74 (d, 1H), | | Method |
| | yl)amino]phenyl}ethyl)propanamide | 7.27 (d, 2H), 4.9 (m, 1H), 3.52 (t, | | 60 |
| | | 2H), 3.21 (s, 3H), 2.35 (t, 2H), 1.3 (d, | | |
| | | 3H) | | |
| 127 | N-(1-(4-(6-(Pyridin-4- | 9.98 (s, 1H), 9.36 (s, 1H), 8.67 (d, | 384 | Method |
| | yl)quinazolin-2- | 2H), 8.41 (s, 1H), 8.25 (m, 2H), | | 19 and |
| | ylamino)phenyl)ethyl)acetamide | 7.90 (d, 2H), 7.83 (d, 2H), 7.75 (d, 1H), | | Method |
| | | 7.27 (d, 2H), 4.88 (m, 1H), 1.83 (s, | | 15 |
| | | 3H), 1.33 (d, 3H) | | |
| 128 | (S)—N-(1-(4-(6-(Pyridin- | 9.98 (s, 1H), 9.36 (s, 1H), 8.67 (d, | 384 | Method |
| | 4-yl)quinazolin-2- | 2H), 8.42 (s, 1H), 8.24 (m, 2H), | | 19 and |
| | ylamino)phenyl)ethyl)acetamide | 7.90 (d, 2H), 7.83 (d, 2H), 7.75 (d, 1H), | | Method |
| | | 7.27 (d, 2H), 4.88 (m, 1H), 1.83 (s, | | 13 |
| | | 3H), 1.33 (d, 3H) | | |
| 129 | (R)—N-(1-(4-(6-(Pyridin- | 9.98 (s, 1H), 9.36 (s, 1H), 8.67 (d, | 384 | Method |
| | 4-yl)quinazolin-2- | 2H), 8.41 (s, 1H), 8.25 (m, 2H), | | 19 and |
| | ylamino)phenyl)ethyl)acetamide | 7.90 (d, 2H), 7.83 (d, 2H), 7.76 (d, 1H), | | Method |
| | | 7.27 (d, 2H), 4.88 (m, 1H), 1.83 (s, | | 14 |
| | | 3H), 1.33 (d, 3H) | | |
| 130¹ | N,P,P-Trimethyl-N-(1- | 9.09 (s, 1H), 9.01 (bs, 1H), 8.52 (d, | 446 | Method |
| | {2-methyl-4-[(6- | 2H), 8.10 (d, 1H), 8.05 (dd, 1H), | | 16 and |
| | pyridin-4-ylquinazolin- | 7.87 (d, 1H), 7.67 (d, 1H), 7.58-7.61 (m, | | Method |
| | 2-yl)amino]phenyl}ethyl)phosphinic | 3H), 7.20 (d, 1H), 5.20 (q, 1H), | | 19 |
| | amide | 2.35 (s, 3H), 2.14 (d, 3H), 1.38 (d, 3H), | | |
| | | 1.22-1.30 (m, 6H) | | |
| 131² | 6-(Pyridin-4-yl)-N-(4- | 9.32 (s, 1H), 9.02 (s, 1H), 8.70 (m, | 396 | Method |
| | (1-(pyrrolidin-1- | 2H), 8.85 (d, 1H), 8.24 (dd, 1H), | | 19 and |
| | yl)ethyl)phenyl)quinazolin- | 8.05 (d, 2H), 7.78-7.85 (m, 3H), 7.38 (d, | | Method |
| | 2-amine | 2H), 3.25 (bs, 1H), 2.58 (bs, 2H), | | 114 |
| | | 2.44 (bs, 2H), 1.75 (bs, 4H), 1.39 (d, | | |
| | | 3H) | | |
| 132³ | N-{4-[(4- | 9.23 (s, 1H), 9.18 (s, 1H), 8.66 (d, | 411 | Method |
| | Methylpiperazin-1- | 2H), 8.25 (d, 1H), 8.19 (dd, 1H), | | 19 and |
| | yl)methyl]phenyl}-6- | 7.97 (d, 2H), 7.81 (d, 1H), 7.74 (d, 2H), | | Method |
| | pyridin-4-ylquinazolin- | 7.29 (d, 2H), 3.47 (s, 2H), 2.41 (bs, | | 115 |
| | 2-amine | 8H), 2.22 (s, 3H) | | |
| 133 | N-{4-[1- | 9.99 (s, 1H), 9.36 (s, 1H), 8.67 (d, | 368 (M − H) | Method |
| | (Dimethylamino)ethyl]phenyl}- | 2H), 8.41 (d, 1H), 8.23 (m, 1H), | | 19 and |
| | 6-pyridin-4- | 7.90 (d, 2H), 7.82 (d, 2H), 7.75 (d, 1H), | | Method |
| | ylquinazolin-2-amine | 7.23 (d, 2H), 3.24 (m, 1H), 2.09 (s, | | 116 |
| | | 6H), 1.27 (d, 3H) | | |
| 134 | 2-[Methyl(1-{4-[(6- | 10.01 (s, 1H), 9.37 (s, 1H), 8.68 (d, | 398 (M − H) | Method |
| | pyridin-4-ylquinazolin- | 2H), 8.42 (d, 1H), 8.25 (m, 1H), | | 19 and |
| | 2-yl)amino]phenyl}ethyl)amino]ethanol | 7.93 (d, 2H), 7.83 (d, 2H), 7.76 (d, 1H), | | Method |
| | | 7.31 (d, 2H), 4.30 (bs, 1H), 3.57 (m, | | 117 |
| | | 1H), 3.46 (m, 2H), 3.26-3.35 (m, 2H), | | |
| | | 2.16 (s, 3H), 1.30 (d, 3H) | | |
| 135 | 3-[Methyl(1-{4-[(6- | 9.99 (s, 1H), 9.36 (s, 1H), 8.67 (d, | 412 (M − H) | Method |
| | pyridin-4-ylquinazolin- | 2H), 8.41 (d, 1H), 8.25 (m, 1H), | | 19 and |
| | 2-yl)amino]phenyl}ethyl)amino]propan- | 7.92 (d, 2H), 7.83 (d, 2H), 7.75 (d, 1H), | | Method |
| | 1-ol | 7.25 (d, 2H), 4.43 (bs, 1H), 3.55 (m, | | 118 |
| | | 1H), 3.39 (m, 2H), 2.03-2.12 (m, 2H), | | |
| | | 2.08 (s, 3H), 1.55 (m, 2H), 1.27 (d, | | |
| | | 3H) | | |
| 136 | N-(4-{1- | 9.96 (s, 1H), 9.36 (s, 1H), 8.67 (d, | 394 (M − H) | Method |
| | [(Cyclopropylmethyl)amino]ethyl}phenyl)- | 2H), 8.41 (d, 1H), 8.24 (m, 1H), | | 19 and |
| | 6- | 7.88 (d, 2H), 7.82 (d, 2H), 7.74 (d, 1H), | | Method |
| | pyridin-4-ylquinazolin- | 7.27 (d, 2H), 3.71 (m, 1H), 2.20 (m, | | 119 |
| | 2-amine | 2H), 1.24 (d, 3H), 0.85 (m, 1H), | | |
| | | 0.34 (m, 2H), 0.01 (m, 2H) | | |
| 137⁴ | N-(6-Pyridin-4- | 10.27 (s, 1H), 9.42 (s, 1H), 8.80 (d, | 314 | Method |
| | ylquinazolin-2- | 2H), 8.56 (d, 1H), 8.35 (dd, 1H), | | 19 and |
| | yl)benzene-1,4-diamine | 8.05-8.10 (m, 4H), 7.81 (d, 1H), | | tert- |
| | | 7.30 (d, 2H) | | butyl-4- |
| | | | | bromophenylcarbamate |
| 138 | N-{4-[1- | (MeOH-d₄) 9.28 (s, 1H), 8.61 (d, | 384 | Method |
| | (Propylamino)ethyl]phenyl}- | 2H), 8.29 (d, 1H), 8.20 (m, 1H), | | 19 and |
| | 6-pyridin-4- | 8.05 (d, 2H), 7.82 (m, 3H), 7.45 (d, 2H), | | Method |
| | ylquinazolin-2-amine | 4.30 (m, 1H), 2.84 (m, 1H), 2.69 (m, | | 120 |
| | | 1H), 1.67 (m, 5H), 0.96 (t, 3H) | | |

¹Compound was prepared by the procedure of Example 123 using microwave conditions at 160° C. for 2400 seconds. Final compound was purified using an ISCO system (EtOAc/MeOH gradient).
²Compound was prepared by the procedure of Example 123 using microwave conditions at 160° C. for 2400 seconds. NMR was taken in acetone-d₆. Compound purified by Gilson HPLC (0.1% ammonium hydroxide in CH₃CN and water).
³Compound was prepared by the procedure of Example 123 using microwave conditions at 160° C. for 2400 seconds. NMR was taken in THF-d₈.
⁴tert-Butyl 4-[(6-pyridin-4-ylquinazolin-2-yl)amino]benzoate was prepared by the procedure of Example 123 using microwave conditions at 160° C. for 2400 seconds. This material was treated with HCl gas in methanol until the reaction mixture indicated complete conversion to Example 137.

### Examples 139 and 140

The two enantiomers of Example 133 were separated using chiral HPLC. A Chiral Pak AD 2 cm×25 cm, 10 um column which was purchased from Chiral Technologies Inc was used. The following conditions were used in the chiral separation: 1:1 ethanol:methanol, 0.1% diethylamine as mobile phase, flow rate of 20 ml/min for 30 minutes at a detector wavelength of 254 nm. Isomer 1 was the first eluting enantiomer and isomer 2 the second eluting enantiomer. Both enantiomers were determined to be >98% ee after chiral separation.

| Ex | Compound | NMR (MeOH-d₄) | m/z |
|---|---|---|---|
| 139 | N-{4-[1- | 9.27 (s, 1H), 8.62 (d, 2H), 8.30 (d, | 368 (M − H) |
| | (Dimethylamino)ethyl]phenyl}- | 1H), 8.20 (m, 1H), 7.86 (m, 5H), | |
| | 6-pyridin-4-ylquinazolin-2- | 7.32 (d, 2H), 3.43 (m, 1H), 2.27 (s, 6H), | |
| | amine (isomer 1) | 1.46 (d, 3H) | |
| 140 | N-{4-[1- | 9.27 (s, 1H), 8.62 (d, 2H), 8.30 (d, | 368 (M − H) |
| | (Dimethylamino)ethyl]phenyl}- | 1H), 8.20 (m, 1H), 7.86 (m, 5H), | |
| | 6-pyridin-4-ylquinazolin-2- | 7.32 (d, 2H), 3.43 (m, 1H), 2.27 (s, 6H), | |
| | amine (isomer 2) | 1.46 (d, 3H) | |

### Examples 141 and 142

The two enantiomers of Example 138 were separated using chiral HPLC. A Chiral Pak AD 2 cm×25 cm, 10 um column which was purchased from Chiral Technologies Inc was used. The following conditions were used in the chiral separation: 1:1 hexane:isopropanol, 0.1% diethylamine as mobile phase, flow rate of 20 ml/min for 40 minutes at a detector wavelength of 254 nm. Isomer 1 was the first eluting enantiomer and isomer 2 the second eluting enantiomer. Both enantiomers were determined to be >98% ee after chiral separation.

| Ex | Compound | NMR (MeOH-d₄) | m/z |
|---|---|---|---|
| 141 | N-{4-[1-(Propylamino)ethyl]phenyl}- | 9.27 (s, 1H), 8.61 (d, 2H), 8.28 (d, | 382 (M − H) |
| | 6-pyridin-4- | 1H), 8.19 (m, 1H), 7.85 (m, 5H), | |
| | ylquinazolin-2-amine | 7.33 (d, 2H), 3.85 (m, 1H), 2.46 (m, 2H), | |
| | (isomer 2) | 1.54 (m, 2H), 1.44 (d, 3H), 0.89 (t, | |
| | | 3H) | |
| 142 | N-{4-[1-(Propylamino)ethyl]phenyl}- | 9.27 (s, 1H), 8.61 (d, 2H), 8.28 (d, | 382 (M − H) |
| | 6-pyridin-4- | 1H), 8.19 (m, 1H), 7.85 (m, 5H), | |
| | ylquinazolin-2-amine | 7.33 (d, 2H), 3.85 (m, 1H), 2.46 (m, 2H), | |
| | (isomer 1) | 1.54 (m, 2H), 1.44 (d, 3H), 0.89 (t, | |
| | | 3H) | |

### Preparation of Starting Materials

### Method 1

### (4-Bromophenyl)acetonitrile

A suspension of 4-bromobenzyl bromide (5.00 g, 0.020 mol) and sodium cyanide (1.18 g, 0.024 mol, 1.2 equiv) in DMF-water (9:1, 35 ml) was stirred at 40° C. for 12 h. The reaction mixture was quenched with water and extracted with EtOAc. The combined organics were dried with NaCl(sat) and then Na₂SO₄(s). The solvents were removed under reduced pressure to give 3.9 g (89%) of the desired product.

### Method 2

### 2-(4-Bromophenyl)-2-methylpropanenitrile

A solution of (4-bromophenyl)acetonitrile (Method 1; 2.1 g, 0.010 mol) in DMSO (20 ml) was treated with sodium hydride (60%, 1.3 g, 0.032 mol, 3 eq). Methyl iodide (2.0 ml, 0.032 mol, 3.0 equiv) was then added dropwise at 0° C. The reaction mixture was stirred at 25° C. for 12 h. The reaction mixture was then quenched with water and extracted with EtOAc. The combined organics were dried with NaCl(sat) and then Na₂SO₄(s). The solvents were removed under reduced pressure. The crude product was purified by column chromatography utilizing an ISCO system (hexane-EtOAc) to give 1.6 g (70%) of the desired product. NMR: 7.62 (d, 2H), 7.47 (d, 2H), 1.66 (s, 6H).

### Method 3

### 4-Bromo-N-(2-methoxyethyl)benzamide

2-Methoxyethylamine (10 ml) at 0° C. was treated with 4-bromobenzoyl chloride (2.0 g, 9.1 mmol). After 15 min, 10% HCl was added to the reaction mixture. The resulting white solid (2.00 g, 85%) was collected by vacuum filtration. NMR: 8.59 (t, 1H), 7.78 (d, 2H), 7.66 (d, 2H), 3.42 (m, 4H), 3.25 (s, 3H).

### Methods 4-17

The following compounds were prepared by the procedure of Method 3 using the appropriate starting materials. If solid did not precipitate upon addition of the 10% HCl, the mixture was extracted with EtOAc, organic layers collected, and concentrated under reduced pressure to afford the crude product which was used directly in the subsequent reaction.

| Method | Compound | m/z | SM |
|---|---|---|---|
| 4 | 4-Bromo-N-(2- | 245 | 4-bromobenzoyl chloride and 2- |
| | hydroxyethyl)benzamide | | aminoethanol |
| 5 | 4-Bromo-N-[2-(2- | 289 | 4-bromobenzoyl chloride and 2-(2- |
| | hydroxyethoxy)ethyl]benzamide | | aminoethoxy)ethanol |
| 6 | 4-Bromo-N-[2- | 272 | 4-bromobenzoyl chloride and N,N- |
| | (dimethylamino)ethyl]benzamide | | dimethylethane-1,2-diamine |
| 7 | 4-Bromo-N-[2- | 286 | 4-bromobenzoyl chloride and N- |
| | (isopropylamino)ethyl]benzamide | | isopropylethane-1,2-diamine |
| 8 | 4-Bromo-N-(2-pyridin-2- | 306 | 4-bromobenzoyl chloride and (2- |
| | ylethyl)benzamide | | pyridin-2-ylethyl)amine |
| 9 | 4-Bromo-N-[2-(1- | 312 | 4-bromobenzoyl chloride and [2-(1- |
| | methylpyrrolidin-2- | | methylpyrrolidin-2-yl)ethyl]amine |
| | yl)ethyl]benzamide | | |
| 10 | 4-Bromo-N-[3- | 286 | 4-bromobenzoyl chloride and N,N- |
| | (dimethylamino)propyl]benzamide | | dimethylpropane-1,3-diamine |
| 11 | 4-Bromo-N-[3-(2-oxopyrrolidin-1- | 326 | 4-bromobenzoyl chloride and 1-(3- |
| | yl)propyl]benzamide | | aminopropyl)pyrrolidin-2-one |
| 12 | N-(2-Methoxyethyl)-4- | 225 | 4-nitrobenzoyl chloride and 2- |
| | nitrobenzamide | | methoxyethylamine |
| 13 | (S)—N-(1-(4- | 243 | (S)-1-(4-bromophenyl)ethanamine and |
| | Bromophenyl)ethyl)acetamide | | acetyl chloride |
| 14 | (R)—N-(1-(4- | 243 | (R)-1-(4-bromophenyl)ethanamine and |
| | Bromophenyl)ethyl)acetamide | | acetyl chloride |
| 15 | N-(1-(4- | 243 | 1-(4-bromophenyl)ethanamine and |
| | Bromophenyl)ethyl)acetamide | | acetyl chloride |
| 16 | N-[1-(4-Bromo-2- | 305 | Method 110 and dimethylphosphinic |
| | methylphenyl)ethyl]-N,P,P- | | chloride |
| | trimethylphosphinic amide | | |
| 17 | N-(1-(4-Bromophenyl)ethyl)-3- | 287 | 1-(4-bromophenyl)ethylamine and 3- |
| | methoxypropanamide | | methoxypropanoyl chloride |

### Method 18

### 6-Bromoquinazolin-2-amine

2-Fluoro-5-bromo benzaldehyde (1.0 g, 4.9 mmol) and guanidine carbonate (1.3 g, 7.4 mmol, 1.5 equiv) were dissolved in DMA and heated to 140° C. for 5 h. The reaction was treated with H₂O and the resulting precipitate was collected by vacuum filtration; m/z 225.

### Method 19

### 6-Pyridin-4-ylquinazolin-2-amine

6-Bromoquinazolin-2-amine (Method 18, 200 mg, 0.89 mmol), pyridin-4-ylboronic acid (165 mg, 1.34 mmol, 1.5 equiv) and K₂CO₃ (370 mg, 2.68 mmol, 3.0 equiv) in DME/H₂O (5:1, 4 ml) were treated with Pd(Ph₃P)₄ (206 mg, 0.179 mmol, 20 mol %). The reaction was stirred at 90° C. for 12 h. The reaction was quenched with 10% NaOH and extracted with EtOAc. The combined organics were dried with NaCl (sat) and then Na₂SO₄(s). The solvents were removed under reduced pressure. The crude product was purified by column chromatography utilizing an ISCO system (EtOAc-MeOH) to give 100 mg (51%) of the desired product; m/z 223.

### Methods 20-24

The following compounds were prepared by the procedure of Method 19 using the appropriate SM.

| Method | Compound | m/z | SM |
|---|---|---|---|
| 20 | 6-(3-Methoxypyridin-4- | 253 | Method 18 and (3-methoxypyridin-4-yl)boronic |
| | yl)quinazolin-2-amine | | acid |
| 21 | 6-(2-Methylpyridin-4- | 237 | Method 18 and (2-methylpyridin-4- |
| | yl)quinazolin-2-amine | | yl)boronic acid |
| 22 | 6-(3-Methylpyridin-4- | 237 | Method 18 and (3-methylpyridin-4- |
| | yl)quinazolin-2-amine | | yl)boronic acid |
| 23 | 6-(3-Chloropyridin-4- | | Method 18 and (3-chloropyridin-4- |
| | yl)quinazolin-2-amine | | yl)boronic acid |
| 24 | 6-(3-Fluoropyridin-4- | | Method 18 and (3-fluoropyridin-4- |
| | yl)quinazolin-2-amine | | yl)boronic acid |

### Method 25

### 3-Bromo-N-(2-methoxyethyl)benzamide

2-Methoxyethylamine (0.435 ml, 5.0 mmol), 3-bromobenzoic acid (1.00 g, 5.0 mmol), and DIPEA (1.31 ml, 7.5 mmol) were dissolved in DMF (10 mL) followed by the addition of HATU (2.85 g, 7.5 mmol). The reaction mixture was stirred for twelve hours at room temperature whereupon the mixture was extracted with saturated NH₄Cl solution and washed with EtOAc three times. The organic layers were dried over magnesium sulfate, filtered, and concentrated in vacuo to afford the crude mixture which after purification using an ISCO system (EtOAc-MeOH) provided the title compound; m/z 259.

### Methods 26-61

The following compounds were prepared by the procedure of Method 25 using the appropriate SM.

| Method | Compound | m/z | SM |
|---|---|---|---|
| 26 | 4-Bromo-N-(2-morpholin-4- | 314 | 4-bromobenzoic acid and (2- |
| | ylethyl)benzamide | | morpholin-4-ylethyl)amine |
| 27 | 4-Bromo-2-chloro-N-(2- | 293 | 4-bromo-2-chlorobenzoic acid |
| | methoxyethyl)benzamide | | and 2-methoxyethylamine |
| 28 | 4-Bromo-2-fluoro-N-(2- | 277 | 4-bromo-2-fluorobenzoic acid |
| | methoxyethyl)benzamide | | and 2-methoxyethylamine |
| 29 | 4-Bromo-N-(2-methoxyethyl)-2- | 273 | 4-bromo-2-methylbenzoic acid |
| | methylbenzamide | | and 2-methoxyethylamine |
| 30 | tert-Butyl {3-[(4-bromobenzoyl)amino]propyl}methylcarbamate | 372 | 4-bromobenzoic acid and tert- |
| | | | butyl (3- |
| | | | aminopropyl)methylcarbamate |
| 31 | tert-Butyl 4-[(4-bromobenzoyl)amino]piperidine- | 384 | 4-bromobenzoic acid and tert- |
| | 1-carboxylate | | butyl 4-aminopiperidine-1- |
| | | | carboxylate |
| 32 | 4-Bromo-N-(tetrahydrofuran-2- | 285 | 4-bromobenzoic acid and |
| | ylmethyl)benzamide | | (tetrahydrofuran-2- |
| | | | ylmethyl)amine |
| 33 | 4-Bromo-N-[(2,2-dimethyl-1,3- | 315 | 4-bromobenzoic acid and [(2,2- |
| | dioxolan-4-yl)methyl]benzamide | | dimethyl-1,3-dioxolan-4- |
| | | | yl)methyl]amine |
| 34 | 4-Bromo-N-methylbenzamide | 215 | 4-bromobenzoic acid and |
| | | | methyl amine |
| 35 | 6-Bromo-N-(2- | 260 | 6-bromonicotinic acid and 2- |
| | methoxyethyl)nicotinamide | | methoxyethylamine |
| 36 | tert-Butyl 2-{[(4-bromobenzoyl)amino]methyl}pyrrolidine- | 384 | 4-bromobenzoic acid and tert- |
| | 1-carboxylate | | butyl 2-(aminomethyl)pyrrolidine- |
| | | | 1-carboxylate |
| 37 | tert-Butyl 2-{[(4-bromobenzoyl)amino]methyl}piperidine- | 398 | 4-bromobenzoic acid and tert- |
| | 1-carboxylate | | butyl 2-(aminomethyl)piperidine- |
| | | | 1-carboxylate |
| 38 | 4-Bromo-N-(3-morpholin-4- | 328 | 4-bromobenzoic acid and (3- |
| | ylpropyl)benzamide | | morpholin-4-ylpropyl)amine |
| 39 | 4-Bromo-N-(2-pyrrolidin-1- | 298 | 4-bromobenzoic acid and (2- |
| | ylethyl)benzamide | | pyrrolidin-1-ylethyl)amine |
| 40 | tert-Butyl 4-{[(4-bromobenzoyl)amino]methyl}piperidine- | 398 | 4-bromobenzoic acid and tert- |
| | 1-carboxylate | | butyl 4-(aminomethyl)piperidine- |
| | | | 1-carboxylate |
| 41 | tert-Butyl {2-[(4- | 344 | 4-bromobenzoic acid and tert- |
| | bromobenzoyl)amino]ethyl}carbamate | | butyl (2-aminoethyl)carbamate |
| 42 | tert-Butyl {3-[(4- | 358 | 4-bromobenzoic acid and tert- |
| | bromobenzoyl)amino]propyl}carbamate | | butyl (3-aminopropyl)carbamate |
| 43 | 4-Bromo-N-(2-piperidin-1- | 312 | 4-bromobenzoic acid and (2- |
| | ylethyl)benzamide | | piperidin-1-ylethyl)amine |
| 44 | 4-Bromo-N,N-dimethylbenzamide | 229 | 4-bromobenzoic acid and N- |
| | | | methylmethanamine |
| 45 | 4-Bromo-N-(2-methoxy-1- | 273 | 4-bromobenzoic acid and (2- |
| | methylethyl)benzamide | | methoxy-1-methylethyl)amine |
| 46 | tert-Butyl {2-[(4-bromobenzoyl)amino]ethyl}methylcarbamate | 358 | 4-bromobenzoic acid and tert- |
| | | | butyl (2-aminoethyl)methylcarbamate |
| 47 | 4-Bromo-2-methyl-N-(2-morpholin-4- | 469 | 4-bromo-2-methylbenzoic acid |
| | ylethyl)benzamide | | and (2-morpholin-4- |
| | | | ylethyl)amine |
| 48 | 4-Bromo-N-(3-hydroxybutyl)benzamide | 273 | 4-bromobenzoic acid and 4- |
| | | | aminobutan-2-ol |
| 49 | 4-Bromo-N-[3-(1H-imidazol-1- | 309 | 4-bromobenzoic acid and [3- |
| | yl)propyl]benzamide | | (1H-imidazol-1- |
| | | | yl)propyl]amine |
| 50 | 4-Bromo-N-[2-(dimethylamino)ethyl]-2- | 286 | 4-bromo-2-methylbenzoic acid |
| | methylbenzamide | | and N,N-dimethylethane-1,2- |
| | | | diamine |
| 51 | 2-Bromo-N-(2-methoxyethyl)isonicotinamide | 260 | 2-bromoisonicotinic acid and |
| | | | (2-methoxyethyl)amine |
| 52 | 6-Bromo-N-(2-methoxyethyl)pyridine- | 260 | 6-bromopyridine-2-carboxylic |
| | 2-carboxamide | | acid and (2- |
| | | | methoxyethyl)amine |
| 53 | tert-Butyl (2-{[(6-bromopyridin-2- | 345 | 6-bromopyridine-2-carboxylic |
| | yl)carbonyl]amino}ethyl)methylcarbamate | | acid and tert-butyl (2- |
| | | | aminoethyl)methylcarbamate |
| 54 | tert-Butyl {2-[(2-bromoisonicotinoyl)amino]ethyl}methylcarbamate | 345 | 2-bromoisonicotinic acid and |
| | | | tert-butyl (2-aminoethyl)methylcarbamate |
| 55 | 4-Bromo-N,N,2-trimethylbenzamide | 243 | 4-bromo-2-methylbenzoic acid |
| | | | and N-methylmethanamine |
| 56 | 4-Bromo-2-methyl-N-(2-piperidin-1- | 326 | 4-bromo-2-methylbenzoic acid |
| | ylethyl)benzamide | | and (2-piperidin-1- |
| | | | ylethyl)amine |
| 57 | 3-Methoxy-N-(2-methoxyethyl)-4- | 255 | 3-methoxy-4-nitrobenzoic acid |
| | nitrobenzamide | | and 2-methoxyethylamine |
| 58 | tert-Butyl {4-[3-methoxypropanoyl)amino]phenyl}carbamate | | tert-butyl (4- |
| | | | aminophenyl)carbamate and 3- |
| | | | methoxypropanoic acid |
| 59 | N-(4-Bromophenyl)-3-methoxy-N- | 273 | 3-methoxypropanoic acid and |
| | methylpropanamide | | (4-bromophenyl)methylamine |
| 60 | N-[(1S)-1-(4-Bromophenyl)ethyl]-3- | 287 | [(1S)-1-(4- |
| | methoxypropanamide | | bromophenyl)ethyl]amine and |
| | | | 3-methoxypropanoic acid |
| 61 | N-[(1R)-1-(4-Bromophenyl)ethyl]-3- | 287 | [(1R)-1-(4- |
| | methoxypropanamide | | bromophenyl)ethyl]amine and |
| | | | 3-methoxypropanoic acid |

### Method 62

### 6-Bromo-N-(4-morpholin-4-ylphenyl)quinazolin-2-amine

6-Bromo-2-chloroquinazoline (prepared in analogy to WO92/15569) (100 mg, 0.412 mmol, 1.0 equiv), (4-morpholin-4-ylphenyl)amine (110 mg, 0.617 mmol, 1.5 equiv), and acetonitrile (5.0 ml) were added to a microwave vial which was heated in a microwave at 125° C. for 30 minutes. The reaction was then concentrated to afford a crude solid which was purified by an ISCO system (100% hexanes to 100% EtOAc) to obtain a yellow solid (117 mg, 74% yield). NMR: 9.78 (s, 1H), 9.21 (s, 1H), 8.13 (s, 1H), 7.77 (m, 3H), 7.52 (d, 1H), 6.94 (d, 2H), 3.72 (m, 4H), 3.03 (m, 4H); m/z 386.

### Methods 63-92

The following compounds were prepared by the procedure of Method 62 using the appropriate SM.

| Method | Compound | m/z | SM |
|---|---|---|---|
| 63 | N-{4-[(6-Bromoquinazolin-2- | 358 | 6-bromo-2-chloroquinazoline |
| | yl)amino]phenyl}acetamide | | and N-(4-aminophenyl)acetamide |
| 64 | 4-[(6-Bromoquinazolin-2- | 317 | 6-bromo-2-chloroquinazoline |
| | yl)amino]phenol | | and 4-aminophenol |
| 65 | 6-Bromo-N-[3- | 369 | 6-bromo-2-chloroquinazoline |
| | (trifluoromethyl)phenyl]quinazolin-2- | | and [3-(trifluoromethyl)phenyl]amine |
| | amine | | |
| 66 | 6-Bromo-N-(3- | 315 | 6-bromo-2-chloroquinazoline |
| | methylphenyl)quinazolin-2-amine | | and m-toluidine |
| 67 | 3-[(6-Bromoquinazolin-2-yl)amino]- | 436 | 6-bromo-2-chloroquinazoline |
| | N-butylbenzenesulfonamide | | and 3-amino-N- |
| | | | butylbenzenesulfonamide |
| 68 | 6-Bromo-N-(3-morpholin-4- | 386 | 6-bromo-2-chloroquinazoline |
| | ylphenyl)quinazolin-2-amine | | and (3-morpholin-4- |
| | | | ylphenyl)amine |
| 69 | 6-Bromo-N-(3- | 359 | 6-bromo-2-chloroquinazoline |
| | isopropoxyphenyl)quinazolin-2-amine | | and (3-isopropoxyphenyl)amine |
| 70 | 6-Bromo-N-(3-methoxy-4- | 345 | 6-bromo-2-chloroquinazoline |
| | methylphenyl)quinazolin-2-amine | | and (3-methoxy-4- |
| | | | methylphenyl)amine |
| 71 | 6-Bromo-N-(1-methyl-1H-pyrazol-3- | 305 | 6-bromo-2-chloroquinazoline |
| | yl)quinazolin-2-amine | | and 1-methyl-1H-pyrazol-3- |
| | | | amine |
| 72 | 6-Bromo-N-(4-piperidin-1- | 384 | 6-bromo-2-chloroquinazoline |
| | ylphenyl)quinazolin-2-amine | | and (4-piperidin-1-ylphenyl)amine |
| 73 | 6-Bromo-N-(6-morpholin-4-ylpyridin- | 387 | 6-bromo-2-chloroquinazoline |
| | 3-yl)quinazolin-2-amine | | and 6-morpholin-4-ylpyridin-3- |
| | | | amine |
| 74 | N-{4-[(6-Bromoquinazolin-2- | 372 | 6-bromo-2-chloroquinazoline |
| | yl)amino]phenyl}-N-methylacetamide | | and N-(4-aminophenyl)-N- |
| | | | methylacetamide |
| 75 | 2-[{4-[(6-Bromoquinazolin-2- | 388 | 6-bromo-2-chloroquinazoline |
| | yl)amino]phenyl}(ethyl)amino]ethanol | | and 2-[(4-aminophenyl)(ethyl)amino]ethanol |
| 76 | 6-Bromo-N-[3-(pyrrolidin-1- | 434 | 6-bromo-2-chloroquinazoline |
| | ylsulfonyl)phenyl]quinazolin-2-amine | | and [3-(pyrrolidin-1- |
| | | | ylsulfonyl)phenyl]amine |
| 77 | 6-Bromo-N-[4-(morpholin-4- | 450 | 6-bromo-2-chloroquinazoline |
| | ylsulfonyl)phenyl]quinazolin-2-amine | | and [4-(morpholin-4- |
| | | | ylsulfonyl)phenyl]amine |
| 78 | 6-Bromo-N-{4-[(difluoromethyl)sulfonyl]phenyl}quinazolin- | 415 | 6-bromo-2-chloroquinazoline |
| | 2-amine | | and {4-[(difluoromethyl)sulfonyl]phenyl}amine |
| 79 | 6-Bromo-N-[4-(pyrrolidin-1- | 434 | 6-bromo-2-chloroquinazoline |
| | ylsulfonyl)phenyl]quinazolin-2-amine | | and [4-(pyrrolidin-1- |
| | | | ylsulfonyl)phenyl]amine |
| 80 | 6-Bromo-N-(4- | 345 | 6-bromo-2-chloroquinazoline |
| | ethoxyphenyl)quinazolin-2-amine | | and (4-ethoxyphenyl)amine |
| 81 | 6-Bromo-N-(3- | 319 | 6-bromo-2-chloroquinazoline |
| | fluorophenyl)quinazolin-2-amine | | and (3-fluorophenyl)amine |
| 82 | 6-Bromo-N-[4-(trifluoromethoxy)phenyl]quinazolin- | 385 | 6-bromo-2-chloroquinazoline |
| | 2-amine | | and [4-(trifluoromethoxy)phenyl]amine |
| 83 | 6-Bromo-N-[3-(piperidin-1- | 448 | 6-bromo-2-chloroquinazoline |
| | ylsulfonyl)phenyl]quinazolin-2-amine | | and [3-(piperidin-1- |
| | | | ylsulfonyl)phenyl]amine |
| 84 | 6-Bromo-N-[3-methoxy-5- | 399 | 6-bromo-2-chloroquinazoline |
| | (trifluoromethyl)phenyl]quinazolin-2- | | and [3-methoxy-5- |
| | amine | | (trifluoromethyl)phenyl]amine |
| 85 | 6-Bromo-N-[3-(morpholin-4- | 450 | 6-bromo-2-chloroquinazoline |
| | ylsulfonyl)phenyl]quinazolin-2-amine | | and [3-(morpholin-4- |
| | | | ylsulfonyl)phenyl]amine |
| 86 | 6-Bromo-N-[4-(methylsulfonyl)phenyl]quinazolin- | 379 | 6-bromo-2-chloroquinazoline |
| | 2-amine | | and [4-(methylsulfonyl)phenyl]amine |
| 87 | 6-Bromo-N-[3-(methylsulfonyl)phenyl]quinazolin- | 379 | 6-bromo-2-chloroquinazoline |
| | 2-amine | | and [3-(methylsulfonyl)phenyl]amine |
| 88 | 6-Bromo-N-(4- | 319 | 6-bromo-2-chloroquinazoline |
| | fluorophenyl)quinazolin-2-amine | | and (4-fluorophenyl)amine |
| 89 | 6-Bromo-N-(4- | 315 | 6-bromo-2-chloroquinazoline |
| | methylphenyl)quinazolin-2-amine | | and p-toluidine |
| 90 | N′-(6-Bromoquinazolin-2-yl)-N,N- | 344 | 6-bromo-2-chloroquinazoline |
| | dimethylbenzene-1,4-diamine | | and N,N-dimethylbenzene-1,4- |
| | | | diamine |
| 91 | 6-Bromo-N-[4-(1H-pyrazol-1- | 367 | 6-bromo-2-chloroquinazoline |
| | yl)phenyl]quinazolin-2-amine | | and [4-(1H-pyrazol-1- |
| | | | yl)phenyl]amine |
| 92 | 6-Bromo-N-(2-methyl-1,3- | 356 | 6-bromo-2-chloroquinazoline |
| | benzoxazol-5-yl)quinazolin-2-amine | | and 2-methyl-1,3-benzoxazol-5- |
| | | | amine |

### Method 93

### 6-Bromo-N-[4-(2-methoxyethoxy)phenyl]quinazolin-2-amine

To [4-(2-methoxyethoxy)phenyl]amine (Method 101; 100 mg, 0.598 mmol) in propan-2-ol (3 ml) was added 6-bromo-2-chloroquinazoline (prepared in analogy to WO92/15569; 131 mg, 0.538 mmol). The reaction mixture was stirred for 2 hours at 100° C. and then allowed to cool to room temperature. The title compound was formed as a precipitate from the solution yielding 123 mg (56% yield); m/z 388.

### Methods 94-98

The following compounds were prepared by the procedure of Method 93, using the appropriate starting materials.

| Method | Compound | m/z | SM |
|---|---|---|---|
| 94 | 4-[(6-Bromoquinazolin-2-yl)amino]-N-(2- | N/A | 6-bromo-2-chloroquinazoline |
| | methoxyethyl)benzenesulfonamide | | and Method 103 |
| 95 | 4-(6-Bromoquinazolin-2-ylamino)-3- | 432 | 6-bromo-2-chloroquinazoline |
| | methoxy-N-(2-methoxyethyl)benzamide | | and Method 105 |
| 96 | 4-[(6-Bromoquinazolin-2-yl)amino]-N-(2- | 403 | 6-bromo-2-chloroquinazoline |
| | methoxyethyl)benzamide | | and Method 104 |
| 97 | N′-(6-Bromoquinazolin-2-yl)-N-(2- | 389 | 6-bromo-2-chloroquinazoline |
| | methoxyethyl)-N-methylbenzene-1,4- | | and Method 107 |
| | diamine | | |
| 98 | N-(4-(6-Bromoquinazolin-2- | 403 | 6-bromo-2-chloroquinazoline |
| | ylamino)phenyl)-3-methoxypropanamide | | and Method 100 |

### Method 99

### [1-(4-Bromophenyl)ethyl](2-methoxyethyl)amine

To a solution of 1-(4-bromophenyl)ethanone (500 mg, 2.51 mmol) and (2-methoxyethyl)amine (188 mg, 2.51 mmol) in toluene (13 ml) was added diethyl 2,6-dimethyl-1,4-dihydropyridine-3,5-dicarboxylate (907 mg, 3.51 mmol), thiourea (19 mg, 0.25 mmol), and 5 Å molecular sieves (˜5 g). The reaction was heated at 50° C. under nitrogen for approximately 40 hours. The reaction mixture was filtered, solvent was evaporated under reduced pressure, and the residue was purified by an ISCO system (EtOAc/Hexane, TLC with I₂) to give 120 mg of a colourless oil (19% yield). NMR (CDCl₃): 7.39 (d, 2H), 7.17 (d, 2H), 3.69 (m, 1H), 3.41 (m, 2H), 3.30 (s, 3H), 2.59 (m, 2H), 1.29 (d, 3H).

### Method 100

### N-(4-Aminophenyl)-3-methoxypropanamide

tert-Butyl {4-[(3-methoxypropanoyl)amino]phenyl}carbamate (Method 58; 744 mgs, 2.53 mmol) was added to TFA (6 ml) and DCM (14.0 ml). The reaction mixture was allowed to stir overnight at room temperature. The solvent was then removed under reduced pressure and redissolved in EtOAc and water. To the water layer was added 4.0 M NaOH and the mixture was extracted with EtOAc (3×). The combined organic extracts were washed with brine and then dried over sodium sulphate. Once the solvent was removed under reduced pressure, a clear oil resulted (68.0 mg, 0.35 mmol) which was used immediately in the next reaction.

### Method 101

### [4-(2-Methoxyethoxy)phenyl]amine

4-Aminophenol (2.2 g, 19.8 mmol) and potassium carbonate (5.5 g, 39.6 mmol) were dissolved in DMF. To the reaction mixture was added 1-chloro-2-methoxyethane (2 ml, 21.8 mmol) and the mixture was stirred overnight at 80° C. The resulting solids were filtered and the filtrate was washed with brine, dried over Na₂SO₄, concentrated under reduced pressure, and purified by an ISCO system (50-100% EtOAc in hexanes) to afford 538 mg of the desired product (16% yield); m/z 168.

### Method 102

### N-(2-Methoxyethyl)-4-nitrobenzenesulfonamide

To a solution of 4-nitrobenzenesulfonyl chloride (5.0 g, 22.6 mmol) and NEt₃ (9.4 ml, 67.7 mmol) in THF was added 2-methoxyethylamine (2.1 ml, 24.8 mmol). The reaction was allowed to stir overnight at room temperature. The resulting white precipitate was filtered and the remaining filtrate was then evaporated under reduced pressure to yield 6.0 g of crude material which was used directly in the next step; m/z 261.

### Method 103

### 4-Amino-N-(2-methoxyethyl)benzenesulfonamide

A solution of N-(2-methoxyethyl)-4-nitrobenzenesulfonamide (Method 102; 1.0 g, 3.8 mmol) and Pd/C (100 mg, 10% by weight) in MeOH was purged 3 times with H₂. The reaction mixture was then stirred for three hours followed by filtration over diatomaceous earth. The filtrate was concentrated under reduced pressure to yield 752 mg (85% yield) of the title compound; m/z 231.

### Methods 104-105

The following compounds were prepared by the procedure of Method 103 using the appropriate starting materials.

| Method | Compound | SM |
|---|---|---|
| 104 | 4-Amino-N-(2-methoxyethyl)benzamide | Method 12 |
| 105 | 4-Amino-3-methoxy-N-(2-methoxyethyl)benzamide | Method 57 |

### Method 106

### N-(2-Methoxyethyl)-N-methyl-4-nitroaniline

1-Bromo-4-nitrobenzene (2.1 g, 10.2 mmol), 2-methoxyethylamine (1.0 ml, 9.3 mmol), Cs₂CO₃ (9.0 g, 27.9 mmol) and BINAP (1.2 g, 1.9 mmol) in dioxane (20 ml) was treated with Pd₂(dba)₃ (853 mg, 0.931 mmol). The reaction mixture was heated to 95° C. overnight. The crude reaction was then filtered and the organic solvents were removed under reduced pressure. The resulting crude residue was purified by an ISCO system (25-100% EtOAc in hexane) to provide 780 mg (40%) of the desired product; m/z 211.

### Method 107

### N-(2-Methoxyethyl)-N-methylbenzene-1,4-diamine

To a solution of N-(2-methoxyethyl)-N-methyl-4-nitroaniline (Method 106; 700 mg, 3.3 mmol) in ethanol (8 ml) was added SnCl.H₂O (1.8 g, 8.3 mmol), and the reaction was stirred overnight at 70° C. To the reaction mixture was then added 4.0 M NaOH. The mixture was extracted (2×) with EtOAc and the combined organic extracts were dried over Na₂SO₄ and concentrated under reduced pressure to afford 505 mg of a green oil which was taken on immediately to the next reaction.

### Method 108

### 1-(4-Bromo-2-methylphenyl)ethanone

4-Bromo-2-methylbenzoic acid (1.18 g, 5.48 mmol) was added to an oven dried 50 mL round bottom flask. The starting material was dissolved in THF (12 ml) and cooled to 0° C. To this solution was added methyllithium (8.56 ml, 13.7 mmol) via syringe over five minutes. After approximately thirty minutes, LCMS indicated consumption of the starting material. The reaction was immediately quenched with saturated aqueous ammonium chloride and partitioned with EtOAc and water. The organic phase was dried over sodium sulfate and purified using an ISCO system (0-10% EtOAc/hexane) to afford the title compound (800 mg, 68% yield), which was taken on directly to the next reaction.

### Method 109

### N-[1-(4-Bromophenyl)ethyl]-3-methoxypropan-1-amine

1-(4-Bromophenyl)ethanone (1.2 g, 6.03 mmol), titanium (IV) isopropoxide (0.883 ml, 3.01 mmol), and (3-methoxypropyl)amine (0.514 ml, 5.02 mmol) were added to dry THF (15 ml) and stirred at room temperature under nitrogen overnight. Sodium borohydride (0.570 g, 15.1 mmol) and dry ethanol (5 ml) were then added and the mixture was stirred at room temperature for another eight hours. The mixture was then poured into aqueous ammonia (2M, 20 ml), filtered, and washed with diethyl ether. The organic phase was separated, and the aqueous layer was extracted with diethyl ether twice. The combined organic layers were extracted with 1M HCl (20 ml). The aqueous layer was washed with diethyl ether and treated with 1M NaOH until reaching pH 12. The basic solution was extracted with diethyl ether, dried over MgSO₄, and concentrated under reduced pressure to afford 858 mg of a colourless oil. The residue was taken on to the next step without further purification; m/z 273.

### Methods 110-120

The following compounds were prepared by the procedure of Method 109, using the appropriate starting materials.

| Method | Compound | m/z | SM |
|---|---|---|---|
| 110¹ | [1-(4-Bromo-2-methylphenyl)ethyl]methylamine | | Method 108 and methanamine |
| 111 | 4-{[1-(4-Bromophenyl)ethyl]amino}butan- | 273 | 1-(4-bromophenyl)ethanone and |
| | 2-ol | | 4-aminobutan-2-ol |
| 112 | 2-{[1-(4-Bromophenyl)ethyl]amino}ethanol | 245 | 1-(4-bromophenyl)ethanone and |
| | | | 2-aminoethanol |
| 113 | 3-{[1-(4-Bromophenyl)ethyl]amino}propan- | 259 | 1-(4-bromophenyl)ethanone and |
| | 1-ol | | 3-aminopropan-1-ol |
| 114¹ | 1-[1-(4-Bromophenyl)ethyl]pyrrolidine | 255 | 1-(4-bromophenyl)ethanone and |
| | | | pyrrolidine |
| 115¹ | 1-(4-Bromobenzyl)-4- | 270 | 4-bromobenzaldehyde and 1- |
| | methylpiperazine | | methylpiperazine |
| 116 | [1-(4-Bromophenyl)ethyl]dimethylamine | 229 | 1-(4-bromophenyl)ethanone and |
| | | | N-methylmethanamine |
| 117 | 2-[[1-(4-Bromophenyl)ethyl](methyl)amino]ethanol | 259 | 1-(4-bromophenyl)ethanone and |
| | | | 2-(methylamino)ethanol |
| 118 | 3-[[1-(4-Bromophenyl)ethyl](methyl)amino]propan- | 273 | 1-(4-bromophenyl)ethanone and |
| | 1-ol | | 3-(methylamino)propan-1-ol |
| 119 | [1-(4-Bromophenyl)ethyl](cyclopropylmethyl)amine | | 1-(4-bromophenyl)ethanone and |
| | | | (cyclopropylmethyl)amine |
| 120 | N-[1-(4-Bromophenyl)ethyl]propan-1- | 243 | 1-(4-bromophenyl)ethanone and |
| | amine | | propan-1-amine |

¹Compound was prepared using the procedure of Method 109 with 1.0 equivalent of bromide, 1.25 equivalents of amine, and 1.25 equivalents of titanium (IV) isopropoxide in MeOH.

1. A compound of formula (I): 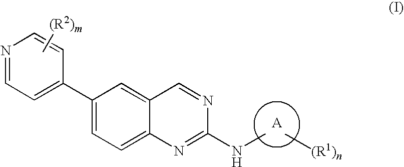
wherein:
Ring A is carbocyclyl or heterocyclyl; wherein if said heterocyclyl contains an —NH— moiety that nitrogen may be optionally substituted by a group selected from R³;
R¹ is a substituent on carbon and is selected from halo, nitro, cyano, hydroxy, trifluoromethoxy, amino, carboxy, carbamoyl, mercapto, sulphamoyl, ureido, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N′—(C₁₋₆alkyl)ureido, N′,N′—(C₁₋₆alkyl)₂ureido, N′—(C₁₋₆alkyl)-N—(C₁₋₆alkyl)ureido, N′,N′—(C₁₋₆alkyl)₂-N—(C₁₋₆alkyl)ureido, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, (R²¹)(R²²)P(O)—, (R²⁹)(R³⁰)P(O)NH—, (R³¹)(R³²)P(O)N(C₁₋₆alkyl)-, (R²⁵)(R²⁶)(R²⁷)Si—, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;
n is selected from 0-4; wherein the values of R¹ may be the same or different;
R² is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;
m is selected from 0-4; wherein the values of R² may be the same or different;
R⁶ and R¹⁰ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)-N—(C₁₋₆alkoxycarbonyl)amino, N—(C₁₋₆alkyl)sulphamoyl, N,N—(C₁₋₆alkyl)₂sulphamoyl, C₁₋₆alkylsulphonylamino, (R²³)(R²⁴)P(O)—, (R³³)(R³⁴)P(O)NH—, (R³⁵)(R³⁶)P(O)N(C₁₋₆alkyl)-, carbocyclyl-R¹²— or heterocyclyl-R¹³—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁴;
R²¹, R²², R²³, R²⁴, R²⁹, R³⁰, R³¹, R³², R³³, R³⁴, R³⁵ and R³⁶ are independently selected from amino, C₁₋₆alkyl, C₁₋₆alkoxy and carbocyclyl;
R²⁵, R²⁶ and R²⁷ are independently selected from hydroxy, C₁₋₆alkyl, C₁₋₆alkoxy and carbocyclyl; or R²⁵ and R²⁶ together with the silicon to which they are attached form a ring; wherein R²⁵, R²⁶ and R²⁷ may be independently optionally substituted on carbon by one or more R²⁸;
R⁴, R⁵, R⁸, R⁹, R¹² and R¹³ are independently selected from a direct bond, —O—, —N(R¹⁶)—, —C(O)—, —N(R¹⁷)C(O)—, —C(O)N(R¹⁸)—, —S(O)ₛ—, —SO₂N(R¹⁹)— or —N(R²⁰)SO₂—; wherein R¹⁶, R¹⁷, R¹⁸, R¹⁹ and R²⁰ are independently selected from hydrogen, C₁₋₆alkoxycarbonyl or C₁₋₆alkyl and s is 0-2;
R³, R⁷, R¹¹ and R¹⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R¹⁵ and R²⁸ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, carbocyclyl or heterocyclyl; wherein if said heterocyclyl contains an —NH— moiety that nitrogen may be optionally substituted by methyl;
or a pharmaceutically acceptable salt thereof.

2. A compound of formula (I), or a pharmaceutically acceptable salt thereof as claimed in claim 1 wherein Ring A is phenyl, pyridyl, 1,3-benzodioxolyl, 2,3-dihydro-1,4-benzodioxinyl, benzooxazolyl or pyrazolyl; wherein said pyrazolyl may be optionally substituted on nitrogen by a group selected from R³; wherein R³ is selected from methyl.

3. A compound of formula (I), or a pharmaceutically acceptable salt thereof as claimed in claim 1 wherein R¹ is a substituent on carbon and is selected from halo, hydroxy, amino, carboxy, C₁₋₆alkyl, C₁₋₆alkoxy, C₁₋₆alkanoyl, N,N—(C₁₋₆alkyl)₂amino, C₁₋₆alkanoylamino, N—(C₁₋₆alkyl)-N—(C₁₋₆alkanoyl)amino, N—(C₁₋₆alkyl)carbamoyl, N,N—(C₁₋₆alkyl)₂carbamoyl, C₁₋₆alkylS(O)ₐ wherein a is 2, C₁₋₆alkoxycarbonyl, N—(C₁₋₆alkyl)sulphamoyl or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷; wherein
R⁶ is selected from halo, cyano, hydroxy, amino, C₁₋₆alkoxy, N—(C₁₋₆alkyl)amino, N,N—(C₁₋₆alkyl)₂amino, C₁₋₆alkanoylamino, C₁₋₆alkoxycarbonylamino, N—(C₁₋₆alkyl)-N—(C₁₋₆alkoxycarbonyl)amino, (R³⁵)(R³⁶)P(O)N(C₁₋₆alkyl)- or heterocyclyl-R¹³—; wherein R⁶ may be optionally substituted on carbon by one or more R¹⁵; and wherein if said heterocyclyl contains an —NH— moiety that nitrogen may be optionally substituted by a group selected from R¹⁴;
R³⁵ and R³⁶ are independently selected from C₁₋₆alkyl;
R⁵ and R¹³ are independently selected from a direct bond, —C(O)—, —C(O)N(R¹⁸)— or —S(O)ₛ—; wherein R¹⁸ is hydrogen and s is 0-2;
R⁷ and R¹⁴ are independently selected from C₁₋₆alkyl, C₁₋₆alkanoyl and C₁₋₆alkoxycarbonyl; and
R¹⁵ is selected from hydroxy, methyl, methoxy, dimethylamino, carbocyclyl or heterocyclyl; wherein if said heterocyclyl contains an —NH— moiety that nitrogen may be optionally substituted by methyl.

4. A compound of formula (I), or a pharmaceutically acceptable salt thereof as claimed in claim 1 wherein n is selected from 0-2; wherein the values of R¹ may be the same or different.

5. A compound of formula (I), or a pharmaceutically acceptable salt thereof as claimed in claim 1 wherein m is 0 or 1.

6. A compound of formula (I), or a pharmaceutically acceptable salt thereof as claimed in claims 1 wherein R² is selected from halo, C₁₋₆alkyl or C₁₋₆alkoxy.

7. A compound of formula (I): 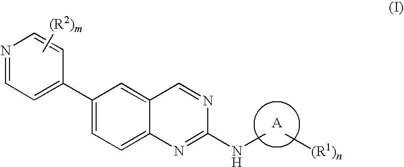
wherein:
Ring A is phenyl, pyrid-2-yl, pyrid-3-yl, 1,3-benzodioxol-5-yl, 2,3-dihydro-1,4-benzodioxin-6-yl, benzooxazol-5-yl or 1-methylpyrazol-3-yl.
R¹ is a substituent on carbon and is selected from
(1R)-1-(3-methoxypropanoylamino)ethyl, (1R)-1-acetamidoethyl,
(1R)-1-dimethylaminoethyl, (1S)-1-(3-methoxypropanoylamino)ethyl, (1S)-1-acetamidoethyl,
(1S)-1-dimethylaminoethyl, (1-tert-butoxycarbonyl-4-piperidyl)methylcarbamoyl,
(1-tert-butoxycarbonylpyrrolidin-2-yl)methylcarbamoyl,
(2,2-dimethyl-1,3-dioxolan-4-yl)methylcarbamoyl, (2-dimethylaminoethylamino)methyl,
(2-hydroxyethyl-methyl-amino)methyl, (2-methoxy-1-methyl-ethyl)carbamoyl,
(2-methoxyethylamino)methyl, (2-methoxyethyl-methyl-amino)methyl,
(2-morpholinoethylamino)methyl, (3S)-3-dimethylaminopyrrolidine-1-carbonyl,
(4-methylpiperazin-1-yl)methyl, (ethyl-(2-hydroxyethyl)amino)methyl,
[(2-dimethylamino-1-methyl-ethyl)amino]methyl,
[2-(1-methylpyrrolidin-2-yl)ethylamino]methyl, 1-(2-hydroxyethylamino)ethyl,
1-(2-hydroxyethyl-methyl-amino)ethyl, 1-(2-methoxyethylamino)ethyl,
1-(3-hydroxybutylamino)ethyl, 1-(3-hydroxypropylamino)ethyl,
1-(3-hydroxypropyl-methyl-amino)ethyl, 1-(3-methoxypropanoylamino)ethyl,
1-(3-methoxypropylamino)ethyl, 1-(cyclopropylmethylamino)ethyl,
1-(dimethylphosphoryl-methyl-amino)ethyl, 1-acetamidoethyl, 1-cyano-1-methyl-ethyl,
1-dimethylaminoethyl, 1-piperidyl, 1-piperidylsulfonyl, 1-propylaminoethyl,
1-pyrrolidin-1-ylethyl, 2-(1-methylpyrrolidin-2-yl)ethylcarbamoyl, 2-(1-piperidyl)ethoxy,
2-(1-piperidyl)ethylcarbamoyl, 2-(2-hydroxyethoxy)ethylcarbamoyl,
2-(2-pyridyl)ethylcarbamoyl, 2-(isopropylamino)ethylcarbamoyl,
2-(tert-butoxycarbonylamino)ethylcarbamoyl, 2,3-dihydroxypropylcarbamoyl,
2-aminoethylcarbamoyl, 2-dimethylaminoethoxy, 2-dimethylaminoethylcarbamoyl,
2-hydroxyethyl, 2-hydroxyethylcarbamoyl, 2-methoxyethoxy, 2-methoxyethylcarbamoyl,
2-methoxyethyl-methyl-amino, 2-methoxyethylsulfamoyl, 2-methylaminoethylcarbamoyl,
2-morpholinoethoxy, 2-morpholinoethylcarbamoyl, 2-oxopyrrolidin-1-yl,
2-piperidylmethylcarbamoyl, 2-pyrrolidin-1-ylethoxy, 2-pyrrolidin-1-ylethylcarbamoyl,
3-(2-oxopyrrolidin-1-yl)propylcarbamoyl,
3-(methyl-tert-butoxycarbonyl-amino)propylcarbamoyl,
3-(tert-butoxycarbonylamino)propylcarbamoyl, 3-aminopropylcarbamoyl,
3-dimethylaminopropylcarbamoyl, 3-dimethylaminopyrrolidine-1-carbonyl,
3-hydroxybutylcarbamoyl, 3-imidazol-1-ylpropylcarbamoyl, 3-methoxypropanoylamino,
3-methoxypropanoyl-methyl-amino, 3-methylaminopropylcarbamoyl, methylsulfonyl,
3-morpholinopropylcarbamoyl, 4-acetylpiperazine-1-carbonyl,
4-methyl-1,4-diazepane-1-carbonyl, 4-methylpiperazine-1-carbonyl, 4-piperidylcarbamoyl,
4-piperidylmethylcarbamoyl, acetamido, acetyl, acetyl-methyl-amino, amino, butylsulfamoyl, carboxy, chloro, formyl, difluoromethylsulfonyl, dimethylamino, dimethylcarbamoyl, ethoxy, ethyl-(2-hydroxyethyl)amino, fluoro, hydroxy, hydroxymethyl, isopropoxy, methoxy, methoxycarbonyl, methyl, methylcarbamoyl, morpholino, morpholinosulfonyl, pyrazol-1-yl, pyrrolidin-1-ylsulfonyl, pyrrolidin-2-ylmethylcarbamoyl, tetrahydrofuran-2-ylmethylcarbamoyl, trifluoromethoxy and trifluoromethyl;
n is selected from 0-2; wherein the values of R¹ may be the same or different;
m is 0 or 1;
R² is selected from fluoro, chloro, methyl or methoxy;
or a pharmaceutically acceptable salt thereof.

8. A compound of formula (I): 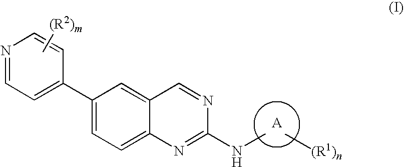
selected from:
N-(2-methoxyethyl)-4-[(6-pyridin-4-ylquinazolin-2-yl)amino]benzamide;
N-(4-{1-[(2-methoxyethyl)amino]ethyl}phenyl)-6-pyridin-4-ylquinazolin-2-amine;
N-methyl-N-{4-[(6-pyridin-4-ylquinazolin-2-yl)amino]phenyl}acetamide;
3-methoxy-N-((1R)-1-{4-[(6-pyridin-4-ylquinazolin-2-yl)amino]phenyl}ethyl)propanamide;
3-methoxy-N-methyl-N-(4-(6-(pyridin-4-yl)quinazolin-2-ylamino)phenyl)propanamide;
3-methoxy-N-((1S)-1-{4-[(6-pyridin-4-ylquinazolin-2-yl)amino]phenyl}ethyl)propanamide;
(S)—N-(1-(4-(6-(pyridin-4-yl)quinazolin-2-ylamino)phenyl)ethyl)acetamide;
(R)—N-(1-(4-(6-(pyridin-4-yl)quinazolin-2-ylamino)phenyl)ethyl)acetamide;
6-(pyridin-4-yl)-N-(4-(1-(pyrrolidin-1-yl)ethyl)phenyl)quinazolin-2-amine;
N-(4-{1-[(cyclopropylmethyl)amino]ethyl}phenyl)-6-pyridin-4-ylquinazolin-2-amine;
N-{4-[(R)-1-(dimethylamino)ethyl]phenyl}-6-pyridin-4-ylquinazolin-2-amine; or
N-{4-[(S)-1-(dimethylamino)ethyl]phenyl}-6-pyridin-4-ylquinazolin-2-amine;
or a pharmaceutically acceptable salt thereof.

9. A process for preparing a compound of formula (I) or a pharmaceutically acceptable salt thereof, as claimed in claim 1, which process comprises of:
Process a) reacting an amine of formula (II): 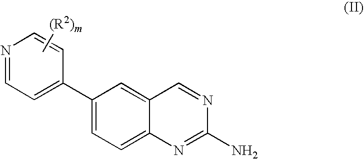
with a compound of formula (III): 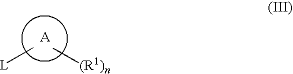
wherein L is a displaceable atom or group; or
Process b) reacting a compound of formula (IV): 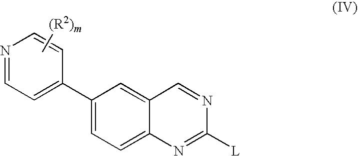
wherein L is a displaceable atom or group; with an amine of formula (V): 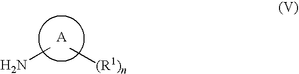
or
Process c) reacting a compound of formula (VI): 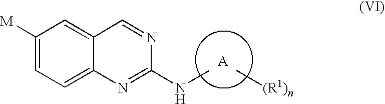
wherein M is an organometallic or organoboron reagent; with a compound of formula (VII): 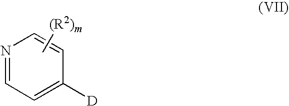
wherein D is a displaceable atom or group; or
Process d) reacting a compound of formula (VIII): 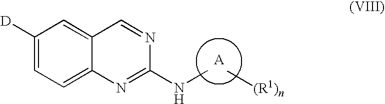
wherein D is a displaceable atom or group; with a compound of formula (IX): 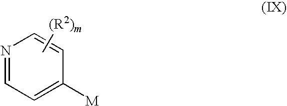
wherein M is an organometallic or organoboron reagent;
and thereafter if necessary:
i) converting a compound of the formula (I) into another compound of the formula (I);
ii) removing any protecting groups;
iii) forming a pharmaceutically acceptable salt.

10. A pharmaceutical composition which comprises a compound of the formula (I), or a pharmaceutically acceptable salt thereof, as claimed in claim 1, in association with a pharmaceutically-acceptable diluent or carrier.

11. A compound of the formula (I), or a pharmaceutically acceptable salt thereof, as claimed in claim 1, for use as a medicament.

12-14. (canceled)

15. A method for producing a B-Raf inhibitory effect in a warm-blooded animal, such as man, in need of such treatment which comprises administering to said animal an effective amount of a compound of formula (I), or a pharmaceutically acceptable salt thereof, as claimed in claim 1.

16. A method for producing an anti-cancer effect in a warm-blooded animal, such as man, in need of such treatment which comprises administering to said animal an effective amount of a compound of formula (I), or a pharmaceutically acceptable salt thereof, as claimed in claim 1.

17. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof, as claimed in claim 1.

18. A pharmaceutical composition which comprises a compound of the formula (I), or a pharmaceutically acceptable salt thereof, as claimed in claim 1, in association with a pharmaceutically-acceptable diluent or carrier for use in the production of a B-Raf inhibitory effect in a warm-blooded animal such as man.

19. A pharmaceutical composition which comprises a compound of the formula (I), or a pharmaceutically acceptable salt thereof, as claimed in claim 1, in association with a pharmaceutically-acceptable diluent or carrier for use in the production of an anti-cancer effect in a warm-blooded animal such as man.

20. A pharmaceutical composition which comprises a compound of the formula (I), or a pharmaceutically acceptable salt thereof, as claimed in claim 1, in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.
